(19) United States
(12) Patent Application Publication
(10) Pub. No.: US 2021/0106574 A1
(43) Pub. Date: Apr. 15, 2021

(54) Methods of Treating Cancer

(71) Applicant: Tesaro, Inc., Waltham, MA (US)

(72) Inventors: Bin Feng, Waltham, MA (US); Sridhar Ramaswamy, Waltham, MA (US); Jing Yu Wang, Waltham, MA (US); Yonghong Xiao, Waltham, MA (US); Yinghui Zhou, Waltham, MA (US)

(21) Appl. No.: 16/958,646

(22) PCT Filed: Dec. 27, 2018
(86) PCT No.: PCT/US2018/067653

§ 371 Date: Jun. 26, 2020

Related U.S. Application Data
(60) Provisional application No. 62/680,511, filed on Jun. 4, 2018.
(60) Provisional application No. 62/613,372, filed on Jan. 3, 2018.
(60) Provisional application No. 62/610,761, filed on Dec. 27, 2017.

(51) Int. Cl. A61K 31/454; A61P 35/00; C07K 16/28

(52) U.S. Cl. CPC A61K 31/454; C07K 16/2827; C07K 16/2818; A61P 35/00

(57) ABSTRACT

The present invention provides methods of treatment of cancer patients having deficiency in at least one non-BRCA1/2 gene involved in the homologous recombination repair (HRR) pathway with a poly(ADP-ribose) polymerase (PARP) inhibitor such as niraparib. In particular, cancer patients having a deficiency in at least one gene selected from the group consisting of BRCA1, BRCA2, ATM, ATR, BAP1, BARD1, BLM, BRIP1, MRE11A, NBN, PALB2, RAD51, RAD51B, RAD51C, RAD51D, RAD52, RAD54L, XRCC2, TP53, or RBI can benefit from treatment with niraparib.

### RELATED APPLICATIONS

The application claims priority to US Provisional Patent Application Nos. 62/610,761, filed Dec. 27, 2017; 62/613,372, filed Jan. 3, 2018; and 62/680,511, filed Jun. 4, 2018, each of which is incorporated by reference herein in its entirety.

### BACKGROUND

Cancer is a serious public health problem, with about 600,920 people in the United States of America expected to die of cancer in 2017 alone, according to the American Cancer Society, Cancer Facts & FIGS. 2016 (https://www.cancer.org/research/cancer-facts-statistics/all-cancer-facts-figures/cancer-facts-figures-2017.html). Accordingly, there continues to be a need for effective therapies to treat cancer patients.

### SUMMARY OF THE INVENTION

Described herein are methods for treating a cancer patient having a deficiency in certain genes involved in the homologous recombination repair (HRR) pathway, including non-BRCA1/2 HRR genes. Further described herein is a poly (ADP-ribose) polymerase (PARP) inhibitor (e.g., as defined herein) for use in methods as defined herein. Further described herein is the use of a poly (ADP-ribose) polymerase (PARP) inhibitor (e.g., as defined herein) in the manufacture of a medicament for use in methods as defined herein. Further described herein is the use of a poly (ADP-ribose) polymerase (PARP) inhibitor (e.g., as defined herein) in methods as defined herein.

In a first aspect, the invention features a method of treating cancer, said method comprising: identifying a cancer patient having deficiency in at least one gene involved in the homologous recombination repair (HRR) pathway, wherein the at least one gene involved in the HRR pathway is not BRCA1 or BRCA2; and administering a poly (ADP-ribose) polymerase (PARP) inhibitor (e.g., niraparib) to said cancer patient. In embodiments, the invention further features a PARP inhibitor for use in the treatment of cancer in a patient identified as having a deficiency in at least one gene involved in the HRR pathway, wherein the at least one gene involved in the HRR pathway is not BRCA1 or BRCA2. In embodiments, said treatment comprising identifying a cancer patient having deficiency in at least one gene involved in the HRR pathway, wherein the at least one gene involved in the HRR pathway is not BRCA1 or BRCA2; and administering said PARP inhibitor (e.g., niraparib) to said cancer patient. In embodiments, the invention further features the use of a PARP inhibitor in the manufacture of a medicament for the treatment of cancer in a patient identified as having a deficiency in at least one gene involved in the HRR pathway, wherein the at least one gene involved in the HRR pathway is not BRCA1 or BRCA2. In embodiments, said treatment comprising identifying a cancer patient having deficiency in at least one gene involved in the HRR pathway, wherein the at least one gene involved in the HRR pathway is not BRCA1 or BRCA2; and administering said PARP inhibitor (e.g., niraparib) to said cancer patient. In embodiments, the invention further features the use of a PARP inhibitor in the treatment of cancer in a patient identified as having a deficiency in at least one gene involved in the HRR pathway, wherein the at least one gene involved in the HRR pathway is not BRCA1 or BRCA2. In embodiments, said treatment comprising identifying a cancer patient having deficiency in at least one gene involved in the HRR pathway, wherein the at least one gene involved in the HRR pathway is not BRCA1 or BRCA2; and administering said PARP inhibitor (e.g., niraparib) to said cancer patient.

In a second aspect, the invention features a method of increasing T cell activation or T cell effector function in a patient having a disorder that is responsive to poly (ADP-ribose) polymerase (PARP) inhibition, said method comprising: identifying said patient, wherein said patient has a deficiency in at least one gene involved in the homologous recombination repair (HRR) pathway, wherein the at least one gene involved in the HRR pathway is not BRCA1 or BRCA2; and administering a PARP inhibitor to said patient. In embodiments, a disorder is cancer. In embodiments, the invention further features a PARP inhibitor for use in a method of increasing T cell activation or T cell effector function in a patient identified as having a disorder that is responsive to PARP inhibition. In embodiments, said method comprises: identifying said patient, wherein said patient has a deficiency in at least one gene involved in HRR pathway, wherein the at least one gene involved in the HRR pathway is not BRCA1 or BRCA2; and administering the PARP inhibitor to said patient. In embodiments, the disorder is cancer. In embodiments, the invention further features the use of a PARP inhibitor in the manufacture of a medicament for use in a method of increasing T cell activation or T cell effector function in a patient identified as having a disorder that is responsive to PARP inhibition. In embodiments, said method comprises: identifying said patient, wherein said patient has a deficiency in at least one gene involved in the HRR pathway, wherein the at least one gene involved in the HRR pathway is not BRCA1 or BRCA2; and administering the PARP inhibitor to said patient. In embodiments, the disorder is cancer. In embodiments, the invention further features the use of a PARP inhibitor in a method of increasing T cell activation or T cell effector function in a patient identified as having a disorder that is responsive to PARP inhibition. In embodiments, said method comprises: identifying said patient, wherein said patient has a deficiency in at least one gene involved in the HRR pathway, wherein the at least one gene involved in the HRR pathway is not BRCA1 or BRCA2; and administering the PARP inhibitor to said patient. In embodiments, the disorder is cancer.

In a third aspect, the invention features a method of reducing tumors or inhibiting the growth of tumor cells in a patient having a disorder that is responsive to poly (ADP-ribose) polymerase (PARP) inhibition, said method comprising: identifying said patient, wherein said patient has a deficiency in at least one gene involved in the homologous recombination repair (HRR) pathway, wherein the at least one gene involved in the HRR pathway is not BRCA1 or BRCA2; and administering a PARP inhibitor to said patient. In embodiments, a disorder is cancer. In embodiments, the invention further features a PARP inhibitor for use in a method of reducing tumors or inhibiting the growth of tumor cells in a patient identified as having a disorder that is responsive to PARP inhibition. In embodiments, said method comprises: identifying said patient, wherein said patient has a deficiency in at least one gene involved in HRR pathway, wherein the at least one gene involved in the HRR pathway is not BRCA1 or BRCA2; and administering the PARP inhibitor to said patient. In embodiments, the disorder is cancer. In embodiments, he invention further features the use of a PARP inhibitor in the manufacture of a medicament for use in a method of reducing tumors or inhibiting the growth of tumor cells in a patient identified as having a disorder that is responsive to PARP inhibition. In embodiments, said method comprises: identifying said patient, wherein said patient has a deficiency in at least one gene involved in the HRR pathway, wherein the at least one gene involved in the HRR pathway is not BRCA1 or BRCA2; and administering the PARP inhibitor to said patient. In embodiments, the disorder is cancer. The invention further features the use of a PARP inhibitor in a method of reducing tumors or inhibiting the growth of tumor cells in a patient identified as having a disorder that is responsive to PARP inhibition. In embodiments, said method comprises: identifying said patient, wherein said patient has a deficiency in at least one gene involved in the HRR pathway, wherein the at least one gene involved in the HRR pathway is not BRCA1 or BRCA2; and administering the PARP inhibitor to said patient. In embodiments, the disorder is cancer.

In a fourth aspect, the invention features a method of inducing an immune response in a patient having a disorder that is responsive to poly (ADP-ribose) polymerase (PARP) inhibition, said method comprising: identifying said patient, wherein said patient has a deficiency in at least one gene involved in the homologous recombination repair (HRR) pathway, wherein the at least one gene involved in the HRR pathway is not BRCA1 or BRCA2; and administering a PARP inhibitor to said patient. In embodiments, an immune response is a humoral or cell mediated immune response. In embodiments, an immune response is a CD4 or CD8 T cell response. In embodiments, an immune response is a B cell response. In embodiments, a disorder is cancer. In embodiments, the invention further features a PARP inhibitor for use in a method of inducing an immune response in a patient identified as having a disorder that is responsive to PARP inhibition. In embodiments, said method comprises: identifying said patient, wherein said patient has a deficiency in at least one gene involved in HRR pathway, wherein the at least one gene involved in the HRR pathway is not BRCA1 or BRCA2; and administering the PARP inhibitor to said patient. In embodiments, the immune response is a humoral or cell mediated immune response. In embodiments, the immune response is a CD4 or CD8 T-cell response. In embodiments, the immune response is a B-cell response. In embodiments, the disorder is cancer. In embodiments, the invention further features the use of a PARP inhibitor in the manufacture of a medicament for use in a method of inducing an immune response in a patient identified as having a disorder that is responsive to PARP inhibition. In embodiments, said method comprises: identifying said patient, wherein said patient has a deficiency in at least one gene involved in the HRR pathway, wherein the at least one gene involved in the HRR pathway is not BRCA1 or BRCA2; and administering the PARP inhibitor to said patient. In embodiments, the immune response is a humoral or cell mediated immune response. In embodiments, the immune response is a CD4 or CD8 T-cell response. In embodiments, the immune response is a B-cell response. In embodiments, the disorder is cancer. In embodiments, the invention further features the use of a PARP inhibitor in a method of inducing an immune response in a patient identified as having a disorder that is responsive to PARP inhibition. In embodiments, said method comprises: identifying said patient, wherein said patient has a deficiency in at least one gene involved in the HRR pathway, wherein the at least one gene involved in the HRR pathway is not BRCA1 or BRCA2; and administering the PARP inhibitor to said patient. In embodiments, the immune response is a humoral or cell mediated immune response. In embodiments, the immune response is a CD4 or CD8 T-cell response. In embodiments, the immune response is a B-cell response. In embodiments, the-cell response. In embodiments, an immune response is a B-cell response. In embodiments, a disorder is cancer.

In a fifth aspect, the invention features a method of enhancing an immune response or increasing the activity of an immune cell in a patient having a disorder that is responsive to poly (ADP-ribose) polymerase (PARP) inhibition, said method comprising: identifying said patient, wherein said patient has a deficiency in at least one gene involved in the homologous recombination repair (HRR) pathway, wherein the at least one gene involved in the HRR pathway is not BRCA1 or BRCA2; and administering a PARP inhibitor to said patient. In embodiments, an immune response is a humoral or cell mediated immune response. In embodiments, an immune response is a CD4 or CD8 T-cell response. In embodiments, an immune response is a B-cell response. In embodiments, a disorder is cancer. The invention further features a PARP inhibitor for use in a method of enhancing an immune response or increasing the activity of an immune cell in a patient identified as having a disorder that is responsive to PARP inhibition. In embodiments, said method comprises: identifying said patient, wherein said patient has a deficiency in at least one gene involved in HRR pathway, wherein the at least one gene involved in the HRR pathway is not BRCA1 or BRCA2; and administering the PARP inhibitor to said patient. In embodiments, the immune response is a humoral or cell mediated immune response. In embodiments, the immune response is a CD4 or CD8 T-cell response. In embodiments, the immune response is a B-cell response. In embodiments, the disorder is cancer. The invention further features the use of a PARP inhibitor in the manufacture of a medicament for use in a method of enhancing an immune response or increasing the activity of an immune cell in a patient identified as having a disorder that is responsive to PARP inhibition. In embodiments, said method comprises: identifying said patient, wherein said patient has a deficiency in at least one gene involved in the HRR pathway, wherein the at least one gene involved in the HRR pathway is not BRCA1 or BRCA2; and administering the PARP inhibitor to said patient. In embodiments, the immune response is a humoral or cell mediated immune response. In embodiments, the immune response is a CD4 or CD8 T-cell response. In embodiments, the immune response is a B-cell response. In embodiments, the disorder is cancer. In embodiments, the invention further features the use of a PARP inhibitor in a method of enhancing an immune response or increasing the activity of an immune cell in a patient identified as having a disorder that is responsive to PARP inhibition. In embodiments, said method comprises: identifying said patient, wherein said patient has a deficiency in at least one gene involved in the HRR pathway, wherein the at least one gene involved in the HRR pathway is not BRCA1 or BRCA2; and administering the PARP inhibitor to said patient. In embodiments, the immune response is a humoral or cell mediated immune response. In embodiments, the immune response is a CD4 or CD8 T-cell response. In embodiments, the immune response is a B-cell response. In embodiments, the cell response. In embodiments, an immune response is a B-cell response. In embodiments, a disorder is cancer.

In a sixth aspect, the invention features a method of treating cancer, said method comprising administering a poly (ADP-ribose) polymerase (PARP) inhibitor (e.g., niraparib) to a cancer patient identified to have deficiency in at least one gene involved in the homologous recombination repair (HRR) pathway, wherein the at least one gene involved in the HRR pathway is not BRCA1 or BRCA2.

In a seventh aspect, the invention features a method of increasing T-cell activation or T-cell effector function in a patient having a disorder that is responsive to poly (ADP-ribose) polymerase (PARP) inhibition, said method comprising administering a PARP inhibitor to said patient, wherein said patient has been identified as having deficiency in at least one gene involved in the homologous recombination repair (HRR) pathway, wherein the at least one gene involved in the HRR pathway is not BRCA1 or BRCA2. In embodiments, a disorder is cancer.

In an eighth aspect, the invention features a method of reducing tumors or inhibiting the growth of tumor cells in a patient having a disorder that is responsive to poly (ADP-ribose) polymerase (PARP) inhibition, said method comprising administering a PARP inhibitor to said patient, wherein said patient has been identified as having deficiency in at least one gene involved in the homologous recombination repair (HRR) pathway, wherein the at least one gene involved in the HRR pathway is not BRCA1 or BRCA2. In embodiments, a disorder is cancer.

In a ninth aspect, the invention features a method of inducing an immune response in a patient having a disorder that is responsive to poly (ADP-ribose) polymerase (PARP) inhibition, said method comprising administering a PARP inhibitor to said patient, wherein said patient has been identified as having deficiency in at least one gene involved in the homologous recombination repair (HRR) pathway, wherein the at least one gene involved in the HRR pathway is not BRCA1 or BRCA2. In embodiments, an immune response is a humoral or cell mediated immune response. In embodiments, an immune response is a CD4 or CD8 T-cell response. In embodiments, an immune response is a B-cell response. In embodiments, a disorder is cancer.

In a tenth aspect, the invention features a method of enhancing an immune response or increasing the activity of an immune cell in a patient having a disorder that is responsive to poly (ADP-ribose) polymerase (PARP) inhibition, said method comprising administering a PARP inhibitor to said patient, wherein said patient has been identified as having deficiency in at least one gene involved in the homologous recombination repair (HRR) pathway, wherein the at least one gene involved in the HRR pathway is not BRCA1 or BRCA2. In embodiments, an immune response is a humoral or cell mediated immune response. In embodiments, an immune response is a CD4 or CD8 T-cell response. In embodiments, an immune response is a B-cell response. In embodiments, a disorder is cancer.

In embodiments, a cancer patient has deficiency in at least one gene selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, and RAD54L, and combinations thereof.

In embodiments, a cancer patient has deficiency in at least one gene selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, RAD54L, TP53, and RB1 and combinations thereof.

In embodiments, a deficiency is in two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more genes selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, and RAD54L.

In embodiments, a deficiency is in two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more, thirty-one or more, or thirty-two or more genes selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, RAD54L, TP53, and RB1.

In embodiments, a cancer patient has a deficiency in a gene panel involved in the HRR pathway, wherein the gene panel comprises TP53 and/or RB1.

In embodiments, a cancer patient has a deficiency in at least one gene involved in the HRR pathway selected from the group consisting of ATM, ATR, BARD1, BRIP1, MRE11A, NBN, PALB2, RAD51, RAD51B, RAD51C, RAD51D, RAD54L, and combinations thereof. In embodiments, a cancer patient has a deficiency in two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L. In embodiments, a cancer patient has a deficiency in each of ATM, ATR, BARD1, BRIP1, MRE11A, NBN, PALB2, RAD51, RAD51B, RAD51C, RAD51D, and RAD54L. In embodiments, a cancer patient has a further deficiency in a gene, where the gene is selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, and FANCE, and combinations thereof.

In embodiments, a cancer patient has a deficiency in at least one gene involved in the HRR pathway selected from the group consisting of ATM, ATR, BAP1, BARD1, BRIP1, MRE11A, NBN, PALB2, RAD51, RAD51B, RAD51C, RAD51D, RAD52, RAD54L, and XRCC2, and combinations thereof. In embodiments, a cancer patient has a deficiency in two or more, three or more, four or more, five or more, seven or more, eight or more, nine or more, ten or more, eleven or more, twelve or more, thirteen or more, fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. In embodiments, a cancer patient has a deficiency in each of ATM, ATR, BAP1, BARD1, BRIP1, MRE11A, NBN, PALB2, RAD51, RAD51B, RAD51C, RAD51D, RAD52, RAD54L, and XRCC2. In embodiments, a cancer patient has a further deficiency in a gene, where the gene is selected from the group consisting of RFC2, XRCC6, POLD2, PCNA, RPA1, RPA2, ERCC3, UNG, ERCC5, MLH1, LIG1, MSH6, POLD4, RFC5, DDB2 ///LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, and FANCE, and combinations thereof.

In embodiments, a cancer patient has a deficiency in at least one gene involved in the HRR pathway selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2, and combinations thereof. In embodiments, a cancer patient has a deficiency in two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a cancer patient has a deficiency in each of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a cancer patient has a further deficiency in a gene, where the gene is selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, and FANCE, and combinations thereof.

In embodiments, a deficiency in the at least one gene involved in the HRR pathway that is not BRCA1 or BRCA2 is identified using a pre-specified HRR gene panel.

In embodiments, a pre-specified HRR gene panel comprises one or more, two or more, three or more, four or more, five or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more genes selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, and RAD54L.

In embodiments, a pre-specified HRR gene panel comprises one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L. In embodiments, a pre-specified HRR gene panel comprises each of ATM, ATR, BARD1, BRIP1, MRE11A, NBN, PALB2, RAD51, RAD51B, RAD51C, RAD51D, and RAD54L. In embodiments, a pre-specified HRR gene panel comprises each of ATM, ATR, BARD1, BRIP1, MRE11A, NBN, PALB2, RAD51, RAD51B, RAD51C, RAD51D, and RAD54L and further comprises BRCA1 and/or BRCA2. In embodiments, a pre-specified HRR gene panel comprises each of ATM, ATR, BARD1, BRIP1, MRE11A, NBN, PALB2, RAD51, RAD51B, RAD51C, RAD51D, RAD54L, BRCA1, and BRCA2. In embodiments, a gene panel further comprises at least one gene selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, and FANCE, and combinations thereof.

In embodiments, a pre-specified HRR gene panel comprises one or more, two or more, three or more, four or more, five or more, seven or more, eight or more, nine or more, ten or more, eleven or more, twelve or more, thirteen or more, fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. In embodiments, a pre-specified HRR gene panel comprises each of ATM, ATR, BAP1, BARD1, BRIP1, MRE11A, NBN, PALB2, RAD51, RAD51B, RAD51C, RAD51D, RAD52, RAD54L, and XRCC2. In embodiments, a pre-specified HRR gene panel comprises each of ATM, ATR, BAP1, BARD1, BRIP1, MRE11A, NBN, PALB2, RAD51, RAD51B, RAD51C, RAD51D, RAD52, RAD54L, and XRCC2, and further comprises BRCA1 and/or BRCA2. In embodiments, a pre-specified HRR gene panel comprises each of ATM, ATR, BAP1, BARD1, BRIP1, MRE11A, NBN, PALB2, RAD51, RAD51B, RAD51C, RAD51D, RAD52, RAD54L, XRCC2, BRCA1, and BRCA2. In embodiments, a gene panel further comprises at least one gene selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, and FANCE, and combinations thereof.

In embodiments, a pre-specified HRR gene panel comprises one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a pre-specified HRR gene panel comprises each of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a pre-specified HRR gene panel comprises each of ATM, ATR, BAP1, BARD1, BLM, BRIP1, MRE11A, NBN, PALB2, RAD51, RAD51B, RAD51C, RAD51D, RAD52, RAD54L, and XRCC2, and further comprises BRCA1 and/or BRCA2. In embodiments, a pre-specified HRR gene panel comprises each of ATM, ATR, BAP1, BARD1, BLM, BRIP1, MRE11A, NBN, PALB2, RAD51, RAD51B, RAD51C, RAD51D, RAD52, RAD54L, and XRCC2, BRCA1, and BRCA2. In embodiments, a gene panel further comprises at least one gene selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, and FANCE, and combinations thereof.

In embodiments, a deficiency in at least one gene involved in the HRR pathway that is not BRCA1 or BRCA2 is a mono-allelic mutation.

In embodiments, at least one of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 has a deficiency caused by a mono-allelic mutation. In embodiments, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 have a deficiency caused by a mono-allelic mutation. In embodiments, each of ATM, ATR, BAP1, BARD1, BLM, BRIP1, MRE11A, NBN, PALB2, RAD51, RAD51B, RAD51C, RAD51D, RAD52, RAD54L, and XRCC2 has a deficiency caused by a mono-allelic mutation. In embodiments, a mono-allelic mutation is independently a germline mutation or a sporadic mutation.

In embodiments, at least one of the genes selected from the group consisting of ATM, ATR, BARD1, BRIP1, MRE11A, NBN, PALB2, RAD51, RAD51B, RAD51C, RAD51D, and RAD54L has a deficiency caused by a mono-allelic mutation. In embodiments, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L have a deficiency caused by a mono-allelic mutation. In embodiments, each of ATM, ATR, BARD1, BRIP1, MRE11A, NBN, PALB2, RAD51, RAD51B, RAD51C, RAD51D, and RAD54L has a deficiency caused by a mono-allelic mutation. In embodiments, a mono-allelic mutation is independently a germline mutation or a sporadic mutation.

In embodiments, at least one of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2 has a deficiency caused by a mono-allelic mutation. In embodiments, two or more, three or more, four or more, five or more, seven or more, eight or more, nine or more, ten or more, eleven or more, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2 have a deficiency caused by a mono-allelic mutation. In embodiments, each of ATM, ATR, BAP1, BARD1, BRIP1, MRE11A, NBN, PALB2, RAD51, RAD51B, RAD51C, RAD51D, RAD52, RAD54L, and XRCC2 has a deficiency caused by a mono-allelic mutation. In embodiments, a mono-allelic mutation is independently a germline mutation or a sporadic mutation.

In embodiments, a deficiency in at least one gene involved in the HRR pathway that is non BRCA1 or BRCA2 is a bi-allelic mutation.

In embodiments, at least one of the genes selected from the group consisting of ATM, ATR, BARD1, BRIP1, MRE11A, NBN, PALB2, RAD51, RAD51B, RAD51C, RAD51D, and RAD54L has a deficiency caused by a bi-allelic mutation. In embodiments, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L have a deficiency caused by a bi-allelic mutation. In embodiments, each of ATM, ATR, BARD1, BRIP1, MRE11A, NBN, PALB2, RAD51, RAD51B, RAD51C, RAD51D, and RAD54L has a deficiency caused by a bi-allelic mutation. In embodiments, a bi-allelic mutation is independently a germline mutation or a sporadic mutation.

In embodiments, at least one of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2 has a deficiency caused by a bi-allelic mutation. In embodiments, two or more, three or more, four or more, five or more, seven or more, eight or more, nine or more, ten or more, eleven or more, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2 have a deficiency caused by a bi-allelic mutation. In embodiments, each of ATM, ATR, BAP1, BARD1, BRIP1, MRE11A, NBN, PALB2, RAD51, RAD51B, RAD51C, RAD51D, RAD52, RAD54L, and XRCC2 has a deficiency caused by a bi-allelic mutation. In embodiments, a bi-allelic mutation is independently a germline mutation or a sporadic mutation.

In embodiments, at least one of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 has a deficiency caused by a bi-allelic mutation. In embodiments, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 have a deficiency caused by a bi-allelic mutation. In embodiments, each of ATM, ATR, BAP1, BARD1, BLM, BRIP1, MRE11A, NBN, PALB2, RAD51, RAD51B, RAD51C, RAD51D, RAD52, RAD54L, and XRCC2 has a deficiency caused by a bi-allelic mutation. In embodiments, a bi-allelic mutation is independently a germline mutation or a sporadic mutation.

In embodiments, a cancer patient has a deficiency in each of the genes selected from the group consisting of ATM, ATR, BARD1, BRIP1, MRE11A, NBN, PALB2, RAD51, RAD51B, RAD51C, RAD51D, and RAD54L. In embodiments, at least one gene having a deficiency has a bi-allelic mutation. In embodiments, each gene having a deficiency has a bi-allelic mutation. In embodiments, at least one gene having a deficiency has a mono-allelic mutation. In embodiments, each gene having a deficiency has a mono-allelic mutation.

In embodiments, a cancer patient has a deficiency in each of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. In embodiments, at least one gene having a deficiency has a bi-allelic mutation. In embodiments, each gene having a deficiency has a bi-allelic mutation. In embodiments, at least one gene having a deficiency has a mono-allelic mutation. In embodiments, each gene having a deficiency has a mono-allelic mutation.

In embodiments, a cancer patient has a deficiency in each of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, at least one gene having a deficiency has a bi-allelic mutation. In embodiments, each gene having a deficiency has a bi-allelic mutation. In embodiments, at least one gene having a deficiency has a mono-allelic mutation. In embodiments, each gene having a deficiency has a mono-allelic mutation.

In embodiments, a deficiency in the at least one gene involved in the HRR pathway (e.g., at least one of ATM, ATR, BAP1, BARD1, BLM, BRIP1, MRE11A, NBN, PALB2, RAD51, RAD51B, RAD51C, RAD51D, RAD52, RAD54L, and XRCC2, and optionally BRCA1 and/or BRCA2) is identified by analyzing cancer cells (e.g., circulating tumor cells). In embodiments, a deficiency in the at least one gene involved in the HRR pathway (e.g., at least one of ATM, ATR, BAP1, BARD1, BLM, BRIP1, MRE11A, NBN, PALB2, RAD51, RAD51B, RAD51C, RAD51D, RAD52, RAD54L, and XRCC2, and optionally BRCA1 and/or BRCA2) is identified by analyzing non-cancer cells. In embodiments, cells (e.g., cancer or non-cancer cells) are obtained from one or more body fluids. In embodiments, cells (e.g., cancer or non-cancer cells) are obtained from blood (e.g., whole blood and/or plasma). In embodiments, cells (e.g., cancer or non-cancer cells) are obtained from saliva, urine, and/or cerebrospinal fluid. In embodiments, cells (e.g., cancer or non-cancer cells) are obtained from one or more tissue samples. In embodiments, the at least one gene involved in the HRR pathway is at least one of ATM, ATR, BARD1, BRIP1, MRE11A, NBN, PALB2, RAD51, RAD51B, RAD51C, RAD51D, and RAD54L and optionally BRCA1 and/or BRCA2. In embodiments, the at least one gene involved in the HRR pathway is at least one of ATM, ATR, BAP1, BARD1, BRIP1, MRE11A, NBN, PALB2, RAD51, RAD51B, RAD51C, RAD51D, RAD52, RAD54L, and XRCC2 and optionally BRCA1 and/or BRCA2.

In embodiments, a deficiency in an at least one gene involved in the HRR pathway (e.g., at least one of ATM, ATR, BAP1, BARD1, BLM, BRIP1, MRE11A, NBN, PALB2, RAD51, RAD51B, RAD51C, RAD51D, RAD52, RAD54L, and XRCC2, and optionally BRCA1 and/or BRCA2) is identified by analyzing cell-free DNA. In embodiments, the at least one gene involved in the HRR pathway is at least one of ATM, ATR, BARD1, BRIP1, MRE11A, NBN, PALB2, RAD51, RAD51B, RAD51C, RAD51D, and RAD54L and optionally BRCA1 and/or BRCA2. In embodiments, the at least one gene involved in the HRR pathway is at least one of ATM, ATR, BAP1, BARD1, BRIP1, MRE11A, NBN, PALB2, RAD51, RAD51B, RAD51C, RAD51D, RAD52, RAD54L, and XRCC2 and optionally BRCA1 and/or BRCA2.

In embodiments, a deficiency in an at least one gene involved in the HRR pathway (e.g., at least one of ATM, ATR, BAP1, BARD1, BLM, BRIP1, MRE11A, NBN, PALB2, RAD51, RAD51B, RAD51C, RAD51D, RAD52, RAD54L, and XRCC2, and optionally BRCA1 and/or BRCA2) is identified by sequencing (e.g., next generation sequencing), PCR, and/or an immunohistochemistry assay. In embodiments, the at least one gene involved in the HRR pathway is at least one of ATM, ATR, BARD1, BRIP1, MRE11A, NBN, PALB2, RAD51, RAD51B, RAD51C, RAD51D, and RAD54L and optionally BRCA1 and/or BRCA2. In embodiments, the at least one gene involved in the HRR pathway is at least one of ATM, ATR, BAP1, BARD1, BRIP1, MRE11A, NBN, PALB2, RAD51, RAD51B, RAD51C, RAD51D, RAD52, RAD54L, and XRCC2 and optionally BRCA1 and/or BRCA2.

In embodiments, a PARP inhibitor is administered in the absence of determining the BRCA status of the patient.

In embodiments, a PARP inhibitor is administered prior to determining the BRCA status of the patient.

In embodiments, a PARP inhibitor is administered independent of the BRCA status of the patient.

In embodiments, the BRCA1 and/or BRCA2 status is determined by including BRCA1 and/or BRCA2 in a pre-specified HRR gene panel (e.g., a panel comprising at least one of ATM, ATR, BAP1, BARD1, BLM, BRIP1, MRE11A, NBN, PALB2, RAD51, RAD51B, RAD51C, RAD51D, RAD52, RAD54L, and XRCC2).

In embodiments, a pre-specified HRR gene panel comprises BRCA1 and/or BRCA2 and further comprises two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L. In embodiments, a pre-specified HRR gene panel comprises BRCA1 and/or BRCA2 and further comprises each of ATM, ATR, BARD1, BRIP1, MRE11A, NBN, PALB2, RAD51, RAD51B, RAD51C, RAD51D, and RAD54L. In embodiments, a pre-specified HRR gene panel comprises BRCA1, BRCA2, ATM, ATR, BARD1, BRIP1, MRE11A, NBN, PALB2, RAD51, RAD51B, RAD51C, RAD51D, and RAD54L.

In embodiments, a pre-specified HRR gene panel comprises BRCA1 and/or BRCA2 and further comprises two or more, three or more, four or more, five or more, seven or more, eight or more, nine or more, ten or more, eleven or more, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. In embodiments, a pre-specified HRR gene panel comprises BRCA1 and/or BRCA2 and further comprises each of ATM, ATR, BAP1, BARD1, BRIP1, MRE11A, NBN, PALB2, RAD51, RAD51B, RAD51C, RAD51D, RAD52, RAD54L, and XRCC2. In embodiments, a pre-specified HRR gene panel comprises BRCA1, BRCA2, ATM, ATR, BAP1, BARD1, BRIP1, MRE11A, NBN, PALB2, RAD51, RAD51B, RAD51C, RAD51D, RAD52, RAD54L, and XRCC2.

In embodiments, a pre-specified HRR gene panel comprises BRCA1 and/or BRCA2 and further comprises two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a pre-specified HRR gene panel comprises BRCA1 and/or BRCA2 and further comprises each of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a pre-specified HRR gene panel comprises BRCA1, BRCA2, ATM, ATR, BAP1, BARD1, BLM, BRIP1, MRE11A, NBN, PALB2, RAD51, RAD51B, RAD51C, RAD51D, RAD52, RAD54L, and XRCC2.

In embodiments, a patient (e.g., a cancer patient) is gBRCA negative, tBRCA negative, or sBRCA negative.

In embodiments, a patient (e.g., a cancer patient) has no germline or sporadic mutation in BRCA1 and no germline or sporadic mutation in BRCA2. In embodiments, a patient (e.g., a cancer patient) has no germline mutation in BRCA1 and/or BRCA2. In embodiments, a patient (e.g., a cancer patient) has no sporadic mutation in BRCA1 and/or BRCA2. In embodiments, a patient (e.g., a cancer patient) has no tumor BRCA1 and/or BRCA2 mutations.

In embodiments, a patient (e.g., a cancer patient) has at least one germline mutation in BRCA1 and/or BRCA2. In embodiments, a patient (e.g., a cancer patient) has at least one sporadic mutation in BRCA1 and/or BRCA2. In embodiments, a patient (e.g., a cancer patient)t has at least one germline or sporadic mutation in BRCA1, and at least one germline or sporadic mutation in BRCA2. In embodiments, a patient (e.g., a cancer patient) has at least one tumor BRCA1 and/or BRCA2 mutation.

In embodiments, a patient (e.g., a cancer patient) is suffering or at risk of a cancer that is adenocarcinoma, adenocarcinoma of the lung, acute myeloid leukemia (“AML”), adrenocortical carcinoma, anal cancer, appendiceal cancer, B-cell derived leukemia, B-cell derived lymphoma, bladder cancer, brain cancer, breast cancer (e.g., triple negative breast cancer (TNBC)), cancer of the fallopian tube(s), cancer of the testes, cerebral cancer, cervical cancer, choriocarcinoma, chronic myelogenous leukemia, colon adenocarcinoma, colon cancer, colorectal cancer, diffuse large B-cell lymphoma (“DLBCL”), endometrial cancer, epithelial cancer, esophageal cancer, Ewing's sarcoma, follicular lymphoma (“FL”), gall bladder cancer, gastric cancer, gastrointestinal cancer, glioma, head and neck cancer, a hematological cancer, hepatocellular cancer, Hodgkin's lymphoma/primary mediastinal B-cell lymphoma, kidney cancer, kidney clear cell cancer, laryngeal cancer, leukemia, liver cancer, lung cancer, lymphoma, melanoma, Merkel cell carcinoma, mesothelioma, monocytic leukemia, multiple myeloma, myeloma, a neuroblastic-derived CNS tumor, non-small cell lung cancer (NSCLC), oral cancer, ovarian cancer, ovarian carcinoma, pancreatic cancer, peritoneal cancer, primary peritoneal cancer, prostate cancer, relapsed or refractory classic Hodgkin's Lymphoma (cHL), renal cell carcinoma, rectal cancer, salivary gland cancer (e.g., a salivary gland tumor), sarcoma, skin cancer, small cell lung cancer, small intestine cancer, squamous cell carcinoma of the anogenital region, squamous cell carcinoma of the esophagus, squamous cell carcinoma of the head and neck (SCHNC), squamous cell carcinoma of the lung, stomach cancer, T-cell derived leukemia, T-cell derived lymphoma, thymic cancer, a thymoma, thyroid cancer, uveal melanoma, urothelial cell carcinoma, uterine cancer, uterine endometrial cancer, uterine sarcoma, vaginal cancer, or vulvar cancer.

In embodiments, a patient (e.g., a cancer patient) is suffering or at risk of a cancer that is endometrial cancer, uterine sarcoma, breast cancer, ovarian cancer, cervical cancer, fallopian tube cancer, primary peritoneal cancer, colon cancer, gastrointestinal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, lung cancer, pancreatic cancer, mesothelioma, sarcoma, or a hematological cancer.

In embodiments, a patient (e.g., a cancer patient) is suffering or at risk of bladder cancer, breast cancer, cancer of the fallopian tube(s), cholagiocarcinoma, colon adenocarcinoma, endometrial cancer, esophageal cancer, Ewing's sarcoma, gastric cancer, kidney clear cell cancer, lung cancer, mesothelioma, ovarian cancer, pancreatic cancer, peritoneal cancer, prostate cancer, uterine endometrial cancer, or uveal melanoma.

In embodiments, a patient (e.g., a cancer patient) is suffering or is at risk of breast cancer or triple negative breast cancer (TNBC).

In embodiments, a patient (e.g., a cancer patient) is suffering or is at risk of lung cancer or non-small cell lung cancer (NSCLC).

In embodiments, a patient (e.g., a cancer patient) is suffering or is at risk of pancreatic cancer.

In embodiments, a patient (e.g., a cancer patient) is suffering or at risk of a gynecological cancer (e.g., ovarian cancer, cervical cancer, fallopian tube cancer, or primary peritoneal cancer).

In embodiments, a patient (e.g., a cancer patient) is suffering or at risk of a recurrent cancer.

In embodiments, a patient (e.g., a cancer patient) has previously been treated with one or more different cancer treatment modalities. In embodiments, a patient (e.g., a cancer patient) has previously been treated with one or more of radiotherapy, chemotherapy, or immunotherapy. In embodiments, a patient (e.g., a cancer patient) has been treated with one, two, three, four, or five lines of prior therapy. In embodiments, a patient (e.g., a cancer patient) has been treated with one or two lines of prior therapy. In embodiments, a patient (e.g., a cancer patient) has been treated with one line of prior therapy. In embodiments, a patient (e.g., a cancer patient) has been treated with two lines of prior therapy. In embodiments, a prior therapy is cytotoxic therapy. In embodiments, a prior therapy is platinum-based chemotherapy.

In embodiments, a patient (e.g., a cancer patient) has undergone at least one cycle of a platinum-based chemotherapy. In embodiments, a patient (e.g., a cancer patient) has undergone at least two cycles of a platinum-based chemotherapy. In embodiments, a cancer is platinum-sensitive. In embodiments, a patient (e.g., a cancer patient) has a complete response or a partial response to the most recent cycle of platinum-based chemotherapy. In embodiments, a patient (e.g., a cancer patient) has a complete response of a partial response to the penultimate cycle of platinum-based chemotherapy. In embodiments, administration of a PARP inhibitor is commenced within 8-weeks of the end of the last cycle of platinum-based chemotherapy. In embodiments, a cancer is recurrent lung cancer (e.g., a recurrent non-small cell lung cancer (NSCLC)). In embodiments, a cancer patient has undergone at least two cycles of a platinum-based chemotherapy. In embodiments, a cancer is platinum-sensitive. In embodiments, a cancer patient has a complete response to the platinum-based chemotherapy. In embodiments, a cancer patient has a partial response to the platinum-based chemotherapy.

In embodiments, a cancer is recurrent ovarian cancer, fallopian tube cancer, or primary peritoneal cancer. In embodiments, a cancer patient has undergone at least one cycle of a platinum-based chemotherapy. In embodiments, a cancer patient has undergone at least two cycles of a platinum-based chemotherapy. In embodiments, a cancer is platinum-sensitive. In embodiments, a cancer patient has a complete response to the platinum-based chemotherapy. In embodiments, a cancer patient has a partial response to the platinum-based chemotherapy. In embodiments, administration of a PARP inhibitor (e.g., niraparib) is commenced within 8-weeks of the end of the last cycle of platinum-based chemotherapy.

In embodiments, a cancer is pancreatic cancer. In embodiments, a cancer patient has undergone at least one cycle of a platinum-based chemotherapy. In embodiments, a cancer patient has undergone at least two cycles of a platinum-based chemotherapy. In embodiments, a cancer is platinum-sensitive. In embodiments, a cancer patient has a complete response to the platinum-based chemotherapy. In embodiments, a cancer patient has a partial response to the platinum-based chemotherapy. In embodiments, administration of a PARP inhibitor (e.g., niraparib) is commenced within 8-weeks of the end of the last cycle of platinum-based chemotherapy.

In embodiments, a PARP inhibitor (e.g., niraparib) is administered daily for at least one 28-day treatment cycle. In embodiments, a PARP inhibitor (e.g., niraparib) is administered daily for at least two, at least three, at least four, at least five at least six, at least seven, at least eight, at least nine, at least ten, at least eleven, at least twelve, or more 28-day treatment cycles. In embodiments, a PARP inhibitor is administered daily for the number of treatment cycles as determined by a physician. In embodiments, a PARP inhibitor (e.g., niraparib) is administered daily for a period sufficient to achieve: i) prolonged progression free survival as compared to control, or ii) a reduced hazard ratio for disease progression or death as compared to control.

In embodiments, a PARP inhibitor (e.g., niraparib) is administered daily for at least one 21-day treatment cycle. In embodiments, a PARP inhibitor (e.g., niraparib) is administered daily for at least two, at least three, at least four, at least five at least six, at least seven, at least eight, at least nine, at least ten, at least eleven, at least twelve, or more 21-day treatment cycles. In embodiments, a PARP inhibitor is administered daily for the number of treatment cycles as determined by a physician. In embodiments, a PARP inhibitor (e.g., niraparib) is administered daily for a period sufficient to achieve: i) prolonged progression free survival as compared to control, or ii) a reduced hazard ratio for disease progression or death as compared to control.

In embodiments, methods described herein further comprise administering one or more additional therapeutic agents in combination with administering a PARP inhibitor (e.g., niraparib).

In embodiments, a one or more additional therapeutic agent is a chemotherapeutic agent. In embodiments, a chemotherapeutic agent is a platinum agent (e.g., cisplatin, carboplatin, oxaliplatin, nedaplatin, triplatin tetranitrate, phenanthriplatin, picoplatin, satraplatin, or the like).

In embodiments, a one or more additional therapeutic agent is an immune checkpoint inhibitor. In embodiments, one, two, or three immune checkpoint inhibitors are administered.

In embodiments, an immune checkpoint inhibitor is an agent that inhibits programmed death-1 protein (PD-1) signaling, T-cell immunoglobulin domain and mucin domain 3 (TIM-3), cytotoxic T-lymphocyte-associated protein 4 (CTLA-4), lymphocyte activation gene-3 (LAG-3), or T-cell immunoglobulin and ITIM domain (TIGIT). In embodiments, an immune checkpoint inhibitor is an antibody.

In embodiments, an immune checkpoint inhibitor is a T-cell immunoglobulin domain and mucin domain 3 (TIM-3) inhibitor. In embodiments, a TIM-3 inhibitor is administered in combination with niraparib.

In embodiments, an immune checkpoint inhibitor is a cytotoxic T-lymphocyte-associated protein 4 (CTLA-4) inhibitor. In embodiments, a CTLA-4 inhibitor is administered in combination with niraparib.

In embodiments, an immune checkpoint inhibitor is a lymphocyte activation gene-3 (LAG-3) inhibitor. In embodiments, a LAG-3 inhibitor is administered in combination with niraparib.

In embodiments, an immune checkpoint inhibitor is a T-cell immunoglobulin and ITIM domain (TIGIT) inhibitor. In embodiments, a TIGIT inhibitor is administered in combination with niraparib.

In embodiments, an immune checkpoint inhibitor is a PD-1 signaling inhibitor. In embodiments, a PD-1 signaling inhibitor is administered in combination with niraparib. In embodiments, a PD-1 signaling inhibitor is administered in combination with a TIM-3 inhibitor and/or a LAG-3 inhibitor. In embodiments, a PD-1 signaling inhibitor is administered in combination with niraparib and a TIM-3 inhibitor. In embodiments, a PD-1 signaling inhibitor is administered in combination with niraparib and a LAG-3 inhibitor. In embodiments, a PD-1 signaling inhibitor is administered in combination with niraparib, a LAG-3 inhibitor, and a TIM-3 inhibitor.

In embodiments, a PD-1 signaling inhibitor is an antibody (e.g., BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, atezolizumab, avelumab, CX-072, durvalumab, FAZ053, LY3300054, PD-L1 millamolecule, or derivatives thereof). In embodiments, a PD-1 signaling inhibitor is an anti-PD-L1/L2 agent. In embodiments, an anti-PD-L1/L2 agent is an antibody (e.g., atezolizumab, avelumab, CX-072, durvalumab, FAZ053, LY3300054, PD-L1 millamolecule, or derivatives thereof).

In embodiments, an immune checkpoint inhibitor (e.g., a PD-1 signaling inhibitor) is administered intravenously.

In embodiments, an immune checkpoint inhibitor (e.g., a PD-1 signaling inhibitor) and a PARP inhibitor (e.g., niraparib) are each administered in 21-day treatment cycles (e.g., each is administered for at least at least one, at least two, at least three, at least four, at least five, at least six, at least seven, at least eight, at least nine, at least ten, at least eleven, at least twelve, or more 21-day treatment cycles). In embodiments an immune checkpoint inhibitor (e.g., a PD-1 signaling inhibitor) and a PARP inhibitor (e.g., niraparib) are administered for the number of treatment cycles as determined by a physician. In embodiments, an immune checkpoint inhibitor (e.g., a PD-1 signaling inhibitor) is administered once during each treatment cycle. In embodiments, an immune checkpoint inhibitor (e.g., a PD-1 signaling inhibitor) is administered on the first day of the first treatment cycle. In embodiments, an immune checkpoint inhibitor (e.g., a PD-1 signaling inhibitor) is administered on the first day of each new treatment cycle or within about three days of the first day of a new treatment cycle. In embodiments, a PARP inhibitor (e.g., niraparib) is administered once daily during a treatment cycle.

In embodiments, an immune checkpoint inhibitor (e.g., a PD-1 signaling inhibitor) and a PARP inhibitor (e.g., niraparib) are each administered in 28-day treatment cycles (e.g., each is administered for at least at least one, at least two, at least three, at least four, at least five, at least six, at least seven, at least eight, at least nine, at least ten, at least eleven, at least twelve, or more 28-day treatment cycles). In embodiments an immune checkpoint inhibitor (e.g., a PD-1 signaling inhibitor) and a PARP inhibitor (e.g., niraparib) are administered for the number of treatment cycles as determined by a physician. In embodiments, an immune checkpoint inhibitor (e.g., a PD-1 signaling inhibitor) is administered once during each treatment cycle. In embodiments, an immune checkpoint inhibitor (e.g., a PD-1 signaling inhibitor) is administered on the first day of the first treatment cycle. In embodiments, an immune checkpoint inhibitor (e.g., a PD-1 signaling inhibitor) is administered on the first day of each new treatment cycle or within about three days of the first day of a new treatment cycle. In embodiments, a PARP inhibitor (e.g., niraparib) is administered once daily during a treatment cycle.

In embodiments, a cancer patient is suffering or is at risk of lung cancer. In embodiments, a lung cancer is non-small cell lung cancer (NSCLC) (e.g., NSCLC characterized by high expression of PD-L1 or characterized by low expression of PD-L1). In embodiments, a lung cancer is squamous NSCLC.

In embodiments, a PARP inhibitor (e.g., niraparib) is administered daily (e.g., as an oral dose). In embodiments, an oral dose is administered in one or more unit dosage forms (e.g., capsules and/or tablets). In embodiments, a PARP inhibitor (e.g., niraparib) is administered daily.

In embodiments, a PARP inhibitor is an agent that inhibits PARP-1 and/or PARP-2. In embodiments, a PARP inhibitor is a small molecule, a nucleic acid, a polypeptide (e.g., an antibody), a carbohydrate, a lipid, a metal, or a toxin. In embodiments, a PARP inhibitor is selected from the group consisting of: ABT-767, AZD 2461, BGB-290, BGP 15, CEP 8983, CEP 9722, DR 2313, E7016, E7449, fluzoparib, IP 4297, INO1001, JPI 289, JPI 547, monoclonal antibody B3-LysPE40 conjugate, MP 124, niraparib, NU 1025, NU 1064, NU 1076, NU1085, olaparib, ONO2231, PD 128763, R 503, R554, rucaparib, SBP 101, SC 101914, simmiparib, talazoparib, veliparib, WW 46, 2-(4-(trifluoromethyl)phenyl)-7,8-dihydro-5H-thiopyrano[4,3-d]pyrimidin-4-ol, and salts or derivatives thereof. In embodiments, a PARP inhibitor is niraparib, olaparib, rucaparib, talazoparib, or veliparib.

In embodiments, a PARP inhibitor is niraparib (e.g., niraparib free base, niraparib tosylate, or niraparib tosylate monohydrate, or any combination thereof).

In embodiments, niraparib is administered daily at an oral dose equivalent to at least 100 mg of niraparib free base. In embodiments, niraparib is administered daily at an oral dose equivalent to about 100 mg of niraparib free base. In embodiments, niraparib is administered daily at an oral dose equivalent to about 200 mg of niraparib free base. In embodiments, the initial dose of niraparib administered to the patient is equivalent to about 200 mg of niraparib free base. In embodiments, niraparib is administered daily at an oral dose equivalent to about 200 mg of niraparib free base when administered in combination with one or more additional therapeutic agents. In embodiments, niraparib is administered daily at an oral dose equivalent to about 300 mg of niraparib free base. In embodiments, methods described herein comprise administering to a patient an oral dose of niraparib equivalent to about 300 mg of niraparib free base for a period of time; and administering niraparib to the patient at a reduced oral dose equivalent to about 200 mg of niraparib free base. In embodiments, an oral dose is administered or provided in one or more unit dosage forms (e.g., capsules and/or tablets). In embodiments, one or more unit dosage forms are capsules. In embodiments, one or more unit dosage forms are tablets. In embodiments, one or more unit dosage forms comprise niraparib in an amount equivalent to about 100 mg of niraparib free base (e.g., an amount of niraparib tosylate monohydrate equivalent to about 100 mg of niraparib free base). In embodiments, an administered form of niraparib comprises niraparib tosylate monohydrate.

In an eleventh aspect, the invention features a method of treating cancer. In embodiments, the method comprises: identifying a cancer patient having deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2; and administering a PARP inhibitor (e.g., niraparib) to said cancer patient. In embodiments, the method comprises identifying a cancer patient having a deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L; and administering a PARP inhibitor (e.g., niraparib) to said cancer patient. In embodiments, a cancer patient has deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2). In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). Ina further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In a twelfth aspect, the invention features a method of increasing T-cell activation or T-cell effector function in a patient having a disorder that is responsive to poly (ADP-ribose) polymerase (PARP) inhibition. In embodiments, the method comprises identifying said patient, wherein said patient has a deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC; and administering a PARP inhibitor (e.g., niraparib) to said patient. In embodiments, the method comprises: identifying said patient, wherein said patient has a deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L; and administering a PARP inhibitor (e.g., niraparib) to said patient. In embodiments, a patient has a deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). In a further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In a thirteenth aspect, the invention features a method of reducing tumors or inhibiting the growth of tumor cells in a patient having a disorder that is responsive to poly (ADP-ribose) polymerase (PARP) inhibition. In embodiments, the method comprises: identifying said patient, wherein said patient has a deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2; and administering a PARP inhibitor (e.g., niraparib) to said patient. In embodiments, the method comprises: identifying said patient, wherein said patient has a deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L; and administering a PARP inhibitor (e.g., niraparib) to said patient. In embodiments, a patient has a deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). In a further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In a fourteenth aspect, the invention features a method of inducing an immune response in a patient having a disorder that is responsive to poly (ADP-ribose) polymerase (PARP) inhibition. In embodiments, the method comprises: identifying said patient, wherein said patient has a deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2; and administering a PARP inhibitor (e.g., niraparib) to said patient. In embodiments, the method comprises: identifying said patient, wherein said patient has a deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 ///LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L; and administering a PARP inhibitor (e.g., niraparib) to said patient. In embodiments, a patient has a deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). Ina further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In a fifteenth aspect, the invention features a method of enhancing an immune response or increasing the activity of an immune cell in a patient having a disorder that is responsive to poly (ADP-ribose) polymerase (PARP) inhibition. In embodiments, the method comprises: identifying said patient, wherein said patient has a deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2; and administering a PARP inhibitor (e.g., niraparib) to said patient. In embodiments, the method comprises: identifying said patient, wherein said patient has a deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L; and administering a PARP inhibitor (e.g., niraparib) to said patient. In embodiments, a patient has a deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). In a further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In a sixteenth aspect, the invention features a method of treating cancer, said method comprising administering a PARP inhibitor (e.g., niraparib) to a cancer patient identified to have deficiency in at least one gene. In embodiments, a cancer patient is identified to have deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient is identified to have deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L. In embodiments, a cancer patient is identified to have deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). In a further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In a seventeenth aspect, the invention features a method of increasing T-cell activation or T-cell effector function in a patient having a disorder that is responsive to poly (ADP-ribose) polymerase (PARP) inhibition, said method comprising administering a PARP inhibitor (e.g., niraparib) to said patient, wherein said patient has been identified as having deficiency in at least one gene. In embodiments, a patient has been identified as having deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a patient has been identified as having deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L. In embodiments, a patient has been identified as having deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). Ina further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In an eighteenth aspect, the invention features a method of reducing tumors or inhibiting the growth of tumor cells in a patient having a disorder that is responsive to poly (ADP-ribose) polymerase (PARP) inhibition, said method comprising administering a PARP inhibitor (e.g., niraparib) to said patient, wherein said patient has been identified as having deficiency in at least one gene. In embodiments, said patient has been identified as having deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, said patient has been identified as having deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L. In embodiments, said patient has been identified to have deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). Ina further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In a nineteenth aspect, the invention features a method of inducing an immune response in a patient having a disorder that is responsive to poly (ADP-ribose) polymerase (PARP) inhibition, said method comprising administering a PARP inhibitor (e.g., niraparib) to said patient, wherein said patient has been identified as having deficiency in at least one gene. In embodiments, said patient has been identified as having deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, said patient has been identified as having deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L. In embodiments, said patient has been identified to have deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). Ina further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In a twentieth aspect, the invention features a method of enhancing an immune response or increasing the activity of an immune cell in a patient having a disorder that is responsive to poly (ADP-ribose) polymerase (PARP) inhibition, said method comprising administering a PARP inhibitor (e.g., niraparib) to said patient, wherein said patient has been identified as having deficiency in at least one gene. In embodiments, said patient has been identified as having deficiency in at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, said patient has been identified as having deficiency in at least one gene that is BRCA1, BRCA2, RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L. In embodiments, said patient has been identified to have deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). In a further aspect, the invention features a PARP inhibitor (e.g., niraparib) for use in said method. In a still further aspect, the invention features the use of a PARP inhibitor (e.g., niraparib) in the manufacture of a medicament for use in said method. In a still further aspect, the invention features the use of a PARP inhibitor (e.g., niraparib) in said method.

In embodiments, a patient (e.g., a cancer patient) has a deficiency in two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L. In embodiments, a patient (e.g., a cancer patient) has a deficiency in each of ATM, ATR, BARD1, BRIP1, MRE11A, NBN, PALB2, RAD51, RAD51B, RAD51C, RAD51D, and RAD54L. In embodiments, has a deficiency in each of ATM, ATR, BARD1, BRIP1, MRE11A, NBN, PALB2, RAD51, RAD51B, RAD51C, RAD51D, and RAD54L, and further has a deficiency in BRCA1 and/or BRCA2. In embodiments, a patient (e.g., a cancer patient) has a further deficiency in at least one gene that is RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, or FANCE.

In embodiments, a patient (e.g., a cancer patient) has a deficiency in two or more, three or more, four or more, five or more, seven or more, eight or more, nine or more, ten or more, eleven or more, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. In embodiments, a patient (e.g., a cancer patient) has a deficiency in each of ATM, ATR, BAP1, BARD1, BRIP1, MRE11A, NBN, PALB2, RAD51, RAD51B, RAD51C, RAD51D, RAD52, RAD54L, and XRCC2. In embodiments, has a deficiency in each of ATM, ATR, BAP1, BARD1, BRIP1, MRE11A, NBN, PALB2, RAD51, RAD51B, RAD51C, RAD51D, RAD52, RAD54L, and XRCC2, and further has a deficiency in BRCA1 and/or BRCA2. In embodiments, a patient (e.g., a cancer patient) has a further deficiency in at least one gene that is RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, or FANCE.

In embodiments, a patient (e.g., a cancer patient) has a deficiency in two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a patient (e.g., a cancer patient) has a deficiency in each of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, has a deficiency in each of ATM, ATR, BAP1, BARD1, BLM, BRIP1, MRE11A, NBN, PALB2, RAD51, RAD51B, RAD51C, RAD51D, RAD52, RAD54L, and XRCC2, and further has a deficiency in BRCA1 and/or BRCA2. In embodiments, a patient (e.g., a cancer patient) has a further deficiency in at least one gene that is RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, or FANCE.

In embodiments, a patient (e.g., a cancer patient) does not have a deficiency in BRCA1 and/or BRCA2. In embodiments, a patient (e.g., a cancer patient) does not have a deficiency in BRCA1 and does not have a deficiency in BRCA2.

In embodiments, the invention features a method of treating recurrent ovarian cancer, fallopian tube cancer, or primary peritoneal cancer, said method comprising identifying a patient (e.g., a cancer patient) having recurrent ovarian cancer, fallopian tube cancer, or primary peritoneal cancer, and having deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2); and administering niraparib to said patient. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). In embodiments, the patient has undergone at least one cycle of platinum-based chemotherapy or at least two cycles of platinum-based chemotherapy. In embodiments, the patient has a complete or partial response to said platinum-based chemotherapy.

In embodiments, the invention features a method of treating non-small cell lung cancer (NSCLC), said method comprising identifying a cancer patient having NSCLC, and having deficiency in at least one gene that is BRCA1, BRCA2, ATM, ATR, BAP1, BARD1, BLM, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BLM, BRIP1, MRE11A, NBN, PALB2, RAD51, RAD51B, RAD51C, RAD51D, RAD52, RAD54L, or XRCC2); and administering niraparib to said cancer patient. In embodiments, a cancer patient has deficiency in at least one gene that is BRCA1, BRCA2, ATM, ATR, BARD1, BRIP1, MRE11A, NBN, PALB2, RAD51, RAD51B, RAD51C, RAD51D, or RAD54L (e.g., at least one gene that is ATM, ATR, BARD1, BRIP1, MRE11A, NBN, PALB2, RAD51, RAD51B, RAD51C, RAD51D, or RAD54L). In embodiments, a cancer patient has deficiency in at least one gene that is BRCA1, BRCA2, ATM, ATR, BAP1, BARD1, BRIP1, MRE11A, NBN, PALB2, RAD51, RAD51B, RAD51C, RAD51D, RAD52, RAD54L, or XRCC2 (e.g., at least one gene that is ATM, ATR, BAP1, BARD1, BRIP1, MRE11A, NBN, PALB2, RAD51, RAD51B, RAD51C, RAD51D, RAD52, RAD54L, or XRCC2). In embodiments, at least one additional therapeutic agent is administered in combination with niraparib. In embodiments, an immune checkpoint inhibitor (e.g., an inhibitor of PD-1 signaling) is administered in combination with niraparib.

In embodiments, a PARP inhibitor (e.g., niraparib) is administered daily (e.g., as an oral dose). In embodiments, an oral dose is administered in one or more unit dosage forms (e.g., capsules and/or tablets). In embodiments, a PARP inhibitor (e.g., niraparib) is administered daily.

In embodiments, a PARP inhibitor is an agent that inhibits PARP-1 and/or PARP-2. In embodiments, a PARP inhibitor is a small molecule, a nucleic acid, a polypeptide (e.g., an antibody), a carbohydrate, a lipid, a metal, or a toxin. In embodiments, a PARP inhibitor is selected from the group consisting of: ABT-767, AZD 2461, BGB-290, BGP 15, CEP 8983, CEP 9722, DR 2313, E7016, E7449, fluzoparib, IP 4297, INO1001, JPI 289, JPI 547, monoclonal antibody B3-LysPE40 conjugate, MP 124, niraparib, NU 1025, NU 1064, NU 1076, NU1085, olaparib, ONO2231, PD 128763, R 503, R554, rucaparib, SBP 101, SC 101914, Simmiparib, talazoparib, veliparib, WW 46, 2-(4-(trifluoromethyl)phenyl)-7,8-dihydro-5H-thiopyrano[4,3-d]pyrimidin-4-ol, and salts or derivatives thereof. In embodiments, a PARP inhibitor is niraparib, olaparib, rucaparib, talazoparib, or veliparib.

In embodiments, a PARP inhibitor is niraparib (e.g., niraparib free base, niraparib tosylate, or niraparib tosylate monohydrate, or any combination thereof).

In embodiments, niraparib is administered daily at an oral dose equivalent to at least 100 mg of niraparib free base. In embodiments, niraparib is administered daily at an oral dose equivalent to about 100 mg of niraparib free base. In embodiments, niraparib is administered daily at an oral dose equivalent to about 200 mg of niraparib free base. In embodiments, the initial dose of niraparib administered to the patient is equivalent to about 200 mg of niraparib free base. In embodiments, niraparib is administered daily at an oral dose equivalent to about 200 mg of niraparib free base when administered in combination with one or more additional therapeutic agents. In embodiments, niraparib is administered daily at an oral dose equivalent to about 300 mg of niraparib free base. In embodiments, methods described herein comprise administering to a patient an oral dose of niraparib equivalent to about 300 mg of niraparib free base for a period of time; and administering niraparib to the patient at a reduced oral dose equivalent to about 200 mg of niraparib free base. In embodiments, an oral dose is administered or provided in one or more unit dosage forms (e.g., capsules and/or tablets). In embodiments, one or more unit dosage forms are capsules. In embodiments, one or more unit dosage forms are tablets. In embodiments, one or more unit dosage forms comprise niraparib in an amount equivalent to about 100 mg of niraparib free base (e.g., an amount of niraparib tosylate monohydrate equivalent to about 100 mg of niraparib free base). In embodiments, an administered form of niraparib comprises niraparib tosylate monohydrate.

### BRIEF DESCRIPTION OF THE DRAWINGS

FIGS. 1A and 1B relate to an exploratory analysis of the NOVA study of maintenance treatment in patients with ovarian cancer. The figures show that niraparib treatment is similarly effective in tBRCA wildtype patients having at least one mutation in a 31 DDR gene panel (FIG. 1A) as compared to tBRCA wildtype patients having no mutation in the 31 DDR gene panel (FIG. 1B).

FIGS. 2A and 2B relate to an exploratory analysis of the NOVA study of maintenance treatment in patients with ovarian cancer. FIG. 2A shows that niraparib treatment is beneficial to patients having a mutation in tBRCA1/2, and FIG. 2B shows that similar benefits are observed in patients having a non-BRCA1/2 mutation in at least one HRR gene.

FIG. 3 shows responses to niraparib based on the tumor growth inhibition (T/C) ratio (T/C % response shown on the X axis). Niraparib sensitivity is observed in PDX models containing ATM, BAP, and BRCA bi-allelic mutations, with responses based on the T/C ratio.

FIGS. 4 and 5 shows evidence of niraparib synthetic lethality by non-BRCA monoallelic and bi-allelic HRR mutations across multiple tumor types using total growth inhibition (TGI). FIG. 4 shows an in vivo screen of HRRmut PDX study (n=87; 17-tumor types) for niraparib monotherapy response (TGI≥100%). FIG. 5 shows an in vitro screen of HRR11 CRISPR/Cas9 KO in isogenic cell lines for niraparib monotherapy response (TGI≥50%). Niraparib sensitivity data using HRR KO isogenic cell lines were consistent with the niraparib sensitivity data observed using HRR mutant PDX models.

FIG. 6 shows 43% of BRCA1/2 bi-allelic mutant PDX models demonstrate moderate sensitivity to niraparib, with ≥50% TGI (80% OvCa PDX models demonstrated>100% TGI).

FIG. 7 shows 33% of ATM bi-allelic mutant NSCLC PDX models showed strong sensitivity to niraparib, with >70% TGI.

FIG. 8 shows BAP bi-allelic mutations are associated with moderate niraparib sensitivity in multiple tumor types. 36% of models (across 5-tumor types) were sensitive to niraparib with ≥50% TGI.

FIG. 9 provides support for treating HRR mutant pancreatic patients with niraparib.

### DETAILED DESCRIPTION OF THE INVENTION

### Definitions

As used herein, the term “administration” typically refers to the administration of a composition to a subject or system. Those of ordinary skill in the art will be aware of a variety of routes that may, in appropriate circumstances, be utilized for administration to a subject, for example a human subject. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In embodiments, administration is oral. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

As used herein, the terms “dosage form” or “unit dosage form” refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a regimen that has been determined to correlate with a desired or beneficial outcome when administered to a relevant population (i.e., with a therapeutic regimen). Those of ordinary skill in the art will appreciate that the total amount of a therapeutic composition or agent administered to a particular subject is determined by one or more attending physicians and may involve administration of multiple dosage forms.

As used herein, the term “regimen” refers to a set of unit doses (typically more than one) that are administered individually to a subject, typically separated by one or more periods of time. In some embodiments, a given therapeutic agent is administered according to a regimen, which may involve one or more doses. In some embodiments, a regimen comprises a plurality of doses each of which is separated in time from other doses. In some embodiments, individual doses are separated from one another by a time period of the same length; in some embodiments, a regimen comprises a plurality of doses, wherein the doses are separated by time periods of different length. In some embodiments, a regimen comprises doses of the same amount. In some embodiments, a regimen comprises doses of different amounts. In some embodiments, a regimen comprises at least one dose, wherein the dose comprises one unit dose of the therapeutic agent. In some embodiments, a regimen comprises at least one dose, wherein the dose comprises two or more unit doses of the therapeutic agent. For example, a dose of 250 mg can be administered as a single 250 mg unit dose or as two 125 mg unit doses. Similarly, a dose of 200 mg can be administered as a single 200 mg unit dose or as two 100 mg unit doses, and a dose of 300 mg can be administered as three 100 mg unit doses. In some embodiments, a regimen is correlated with or result in a desired or beneficial outcome when administered across a relevant population (i.e., is a therapeutic regimen). For example, a regimen can result in: (i) prolonged progression free survival as compared to control; (ii) a reduced hazard ratio for disease progression or death as compared to control; and/or (iii) prolonged overall survival as compared to control, or iv) an overall response rate of at least 30%.

As used herein, the term “patient”, “subject”, or “test subject” are used interchangeable throughout, and refers to any organism to which the provided compound or compounds described herein are administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.). In embodiments, a subject is a human. In some embodiments, a subject may be suffering from, and/or susceptible to a disease, disorder, and/or condition (e.g., any of the cancers described herein, including cancers such as ovarian cancer, cancer of the fallopian tube(s), peritoneal cancer, breast cancer, pancreatic cancer, lung cancer, and non-small cell lung cancer (NSCLC). In some embodiments, the patient is a human patient possessing one or more female reproductive organs. In some embodiments, the patient is a human female patient (i.e., a woman) that has been diagnosed with a gynecological cancer (e.g., cancer such as ovarian cancer, cancer of the fallopian tube(s), peritoneal cancer, and breast cancer). In some embodiments, the patient is a human patient that has been diagnosed with a lung cancer (e.g., non-small cell lung cancer). In some embodiments, the patient is a human that has been diagnosed with pancreatic cancer. As used herein, a “patient population” or “population of subjects” refers to a plurality of patients or subjects.

As used herein, a “therapeutically effective amount” refers to an amount of a therapeutic agent that produces the desired effect for which it is administered. In some embodiments, the term refers to an amount that is sufficient, when administered to a population suffering from or susceptible to a disease, disorder, and/or condition in accordance with a regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a regimen.

As used herein, a “chemotherapeutic agent” refers to a chemical agent that inhibits the proliferation, growth, life-span, and/or metastatic activity of cancer cells. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

As used herein, “CA-125” means cancer antigen 125. A CA-125 test may be used to measure the amount of the protein CA-125 in the blood of a patient. A CA-125 test may be used to monitor certain cancers during and after treatment, including use to evaluate prolongation of progression free survival. In some cases, a CA-125 test may be used to look for early signs of ovarian cancer in women with a very high risk of the disease.

As used herein, “homologous recombination” refers to a process wherein nucleotide sequences between distinct stands of DNA are exchanged. Homologous recombination is involved in a number of different biological processes, for example, homologous recombination occurs as part of the DNA repair process (e.g., doubled-strand break repair pathway and synthesis-dependent strand annealing pathway) and during process of meiosis/gametogenesis of eukaryotic organisms. As used herein, “homologous recombination deficiency”, “homologous recombination repair deficiency”, “RR”, “homologous repair deficiency”, or “HRD” refers to a reduction or impairment of the homologous recombination process. Without wishing to be bound by theory, it is believed that since homologous recombination is involved in DNA repair, a homologous recombination deficient sample would be unable or have a reduced ability to repair DNA damage such as double-strand breaks. As such, a sample that is HRD would accumulate genomic errors or chromosomal aberrations can be used as a biomarker for HRD. As used herein, “chromosomal aberration” or “CA” refers to a detectable variation in a sample's chromosomal DNA. In some embodiments, CA may fall into at least one of three overlapping categories: loss of heterozygosity (LOH), allelic imbalance (e.g., telomeric allelic imbalance (TAI)), or large scale transition (LST). In some embodiments, “HRD status” is determined by the detection of CA in a sample (e.g., a tumor sample) obtained from a patient. In some embodiments, a positive HRD status refers to when a sample obtained from a patient meets a threshold number or level of CAs at a specified number of chromosomal indicator regions. In some embodiments, HRD status is determined using a commercially available diagnostic to detect chromosomal aberrations in a sample (e.g. a tumor sample) and/or to assess if a sample is unable to repair double-strand DNA breaks. Commercially available diagnostics to assess HRD status include the myChoice HRD™ diagnostic kit.

As used herein, loss of heterozygosity (LOH) refers to the change from heterozygosity to homozygosity a polymorphic loci of interest. Polymorphic loci within the human genome (e.g., single nucleotide polymorphisms (SNPs)) are generally heterozygous within an individual's germline since that individual typically receives one copy from the biological father and one copy from the biological mother. Somatically, however, this heterozygosity can change (via mutation) to homozygosity, referred to herein as LOH. LOH may result from several mechanisms. For example, in some cases, a locus of one chromosome can be deleted in a somatic cell. The locus that remains present on the other chromosome (the other non-sex chromosome for males) is an LOH locus as there is only one copy (instead of two copies) of that locus present within the genome of the affected cells. This type of LOH event results in a copy number reduction. In other cases, a locus of one chromosome (e.g., one non-sex chromosome for males) in a somatic cell can be replaced with a copy of that locus from the other chromosome, thereby eliminating any heterozygosity that may have been present within the replaced locus. In such cases, the locus that remains present on each chromosome is an LOH locus and can be referred to as a copy neutral LOH locus. LOH and its use in determining HRD is described in detail in International Application No. PCT/US2011/040953 (published as WO/2011/160063), the entire contents of which are incorporated herein by reference.

A broader class of chromosomal aberration, which encompasses LOH, is allelic imbalance. Allelic imbalance occurs when the relative copy number (i.e., copy proportion) at a particular locus in somatic cells differs from the germline. For example, if the germline has one copy of allele A and one copy of allele B at a particular locus and a somatic cell has two copies of A and one copy of B, there is allelic imbalance at the locus because the copy proportion of the somatic cell (2:1) differs from the germline (1:1). LOH is an example of allelic imbalance since the somatic cell has a copy proportion (1:0 or 2:0) that differs from the germline (1:1). Allelic imbalance also encompasses more types of chromosomal aberration, e.g., 2:1 germline going to 1:1 somatic; 1:0 germline going to 1:1 somatic; 1:1 germline going to 2:1 somatic, etc. Analysis of regions of allelic imbalance encompassing the telomeres of chromosomes is particularly useful in the invention. Thus, a “telomeric allelic imbalance region” or “TAI Region” is defined as a region with allelic imbalance that (a) extends to one of the subtelomeres and (b) does not cross the centromere. TAI and its use in determining HRD is described in detail in International Application No. PCT/US2011/048427 (published as WO/2012/027224), the entire contents of which are incorporated herein by reference.

A class of chromosomal aberrations that is broader still, which encompasses LOH and TAI, is referred to herein as large scale transition (“LST”). LST refers to any somatic copy number transition (i.e., breakpoint) along the length of a chromosome where it is between two regions of at least some minimum length (e.g., at least 3, 4, 5, 6, 7, 8 9, 10, 11 12, 13, 14, 15, 16, 17, 18, 19, or 20 or more megabases) after filtering out regions shorter than some maximum length (e.g., 0.1, 0.2, 0.3, 0.4, 0.5, 0.6, 0.7, 0.8, 0.9, 1, 1.5, 2, 2.5, 3, 3.5, 4 or more megabases). For example, if after filtering out regions shorter than 3 megabases the somatic cell has a copy number of 1:1 for, e.g., at least 10 megabases and then a breakpoint transition to a region of, e.g., at least 10 megabases with copy number 2:2, this is an LST. An alternative way of defining the same phenomenon is as an LST Region, which is genomic region with stable copy number across at least some minimum length (e.g., at least 3, 4, 5, 6, 7, 8, 9, 10, 11 12, 13, 14, 15, 16, 17, 18, 19, or 20 megabases) bounded by breakpoints (i.e., transitions) where the copy number changes for another region also at least this minimum length. For example, if after filtering out regions shorter than 3 megabases the somatic cell has a region of at least 10 megabases with copy number of 1:1 bounded on one side by a breakpoint transition to a region of, e.g., at least 10 megabases with copy number 2:2, and bounded on the other side by a breakpoint transition to a region of, e.g., at least 10 megabases with copy number 1:2, then this is two LSTs. Notice that this is broader than allelic imbalance because such a copy number change would not be considered allelic imbalance (because the copy proportions 1:1 and 2:2 are the same, i.e., there has been no change in copy proportion). LST and its use in determining HRD is described in detail in Popova et al., “Ploidy and large-scale genomic instability consistently identify basal-like breast carcinomas with BRCA1/2 inactivation”, Cancer Res. (2012) 72:5454-62.

As used herein, “BRCA mutation” or “mutation of BRCA” refers to a change or difference in the sequence of at least one copy of either or both of the BRCA1 or BRCA2 genes relative to an appropriate reference sequence (e.g., a wild type reference and/or a sequence that is present in non-cancerous cells in the subject). A mutation in the BRCA1/2 gene may result in a BRCA1/2 deficiency, which may include, for example a loss or reduction in the expression or function of the BRCA gene and/or encoded protein. Such mutations may also be referred to as “deleterious mutations” or may be suspected to be deleterious mutations. A BRCA mutation can be a “germline BRCA mutation,” which indicates it was inherited from one or both parents. Germline mutations affect every cell in an organism and are passed on to offspring. A BRCA mutation can also be acquired during one's lifetime, i.e. spontaneously arising in any cell in the body (“soma”) at any time during a patient's life, (i.e., non-inherited), which is interchangeably referred to herein as a “sporadic BRCA mutation” or a “somatic BRCA mutation”. Genetic tests are available, and known by those of skill in the art. For example, the BRACAnalysis CDx® kit is an in vitro diagnostic for detection and classification of BRCA1/2 variants. Using isolated genomic DNA, the BRACAnalysis CDx identifies mutations in the protein coding regions and intron/exon boundaries of the BRCA1 and BRCA2 genes. Single nucleotide variants and small insertions and deletions (indels) may be identified by polymerase chain reaction (PCR) and nucleotide sequencing. Large deletions and duplications in BRCA1 and BRCA2 may be detected using multiplex PCR. Indication of a “BRCA status” refers to, in at least some cases, whether a mutation is present in at least one copy of either BRCA1 or BRCA2. In some embodiments, indication of a BRCA status may refer to the mRNA expression level, methylation level or other epigenetic modification of either or both of BRCA1 and BRCA2. In some embodiments, a patient with a “positive BRCA status” refers to a patient from whom a sample has been determined to contain a mutation in BRCA1 and/or BRCA2. In some embodiments, a positive BRCA status refers to the presence of either a germline BRCA mutation (gBRCA^{mut}) or a somatic BRCA mutation (sBRCA^{mut}). In some embodiments, a patient with a “positive BRCA status” refers to a patient from whom a sample has been determined to have a reduced expression of BRCA1 and/or BRCA2. In some embodiments, BRCA status is determined for germline BRCA mutations (e.g., gBRCA^{mut}) and is performed on a blood sample of a subject. In some embodiments, BRCA status is determined for somatic BRCA mutations (sBRCA^{mut}) or total BRCA mutations (tBRCA^{mut}, which includes both somatic and BRCA germline mutations).

As used herein, the term “genes involved in DNA repair” means any gene involved in repair of DNA in the cell. Table 1 and Table 2 each list a representative set of genes involved in DNA repair. These include genes involved in homologous recombination (“HR”), which is genetic recombination in which nucleotide sequences are exchanged between two similar or identical molecules of DNA. HR is most widely used by cells to accurately repair harmful breaks that occur on both strands of DNA (HRR pathway for DNA repair), known as double-strand breaks. Genes involved in the HRR pathway include ATM, ATR, BAP1, BARD1, BLM, BRIP1, MRE11A, NBN, PALB2, RAD51, RAD51B, RAD51C, RAD51D, RAD52, RAD54L, and XRCC2, as well as BRCA1 and BRCA2. One of skill in the art will be able to determine whether a gene is involved in DNA repair and in particular DNA repair pathways (e.g., the HRR pathway). DNA repair status refers to the presence or absence of mutations in one or more of a gene involved in DNA repair. In certain embodiments, the invention involves use of a PARP inhibitor to treat a cancer patient regardless of DNA repair status.

As used herein, “HRR gene mutation” or “mutation of a HRR gene,” refers to a change or difference in the sequence of at least one copy of a gene that is involved in the HRR pathway for DNA repair (e.g., any of ATM, ATR, BAP1, BARD1, BLM, BRIP1, MRE11A, NBN, PALB2, RAD51, RAD51B, RAD51C, RAD51D, RAD52, RAD54L, and XRCC2) relative to an appropriate reference sequence (e.g., a wild type reference and/or a sequence that is present in non-cancerous cells in the subject). A mutation of a HRR gene can result in a HRR gene deficiency, which may include, for example, a loss or reduction in the expression or function of the mutated gene and/or encoded protein. Such mutations may also be referred to as “deleterious mutations” or may be suspected to be deleterious mutations. A HRR gene mutation can be a “germline HRR gene mutation”, which indicates it was inherited from one or both parents. Germline gene mutations affect every cell in an organism and are passed on to offspring. An HRR gene mutation can also be acquired during one's lifetime, i.e. spontaneously arising in any cell in the body (“soma”) at any time during the patient's life, (i.e., non-inherited), which is referred to herein as a “sporadic HRR gene mutation” or a “somatic HRR gene mutation” interchangeably. HRR gene mutations can be identified using methods known in the art (e.g., the methods described herein). For example, isolated genomic DNA can be used to identify mutations in the protein coding regions and intron/exon boundaries of an HRR gene. Single nucleotide variants and small insertions and deletions (indels) may be identified by polymerase chain reaction (PCR) and nucleotide sequencing. Large deletions and duplications in an HRR gene may be detected using multiplex PCR. An HRR gene mutation can be a bi-allelic (homozygous) mutation, in which a mutation is found in both alleles of the gene. A mono-allelic (heterozygous) HRR gene mutation is found in one allele of the gene.

As used herein, the term “PARP inhibitor” means an agent that inhibits the activity or decreases the function of any one of the poly(ADP-ribose) polymerase (PARP) family of proteins. This may include inhibitors of any one of more of the over 15 different enzymes in the PARP family, which engage in a variety of cellular functions, including cell cycle regulation, transcription, and repair of DNA damage. In embodiments, a PARP inhibitor inhibits PARP-1 and/or PARP-2.

As used herein, the term “progression free survival” means the time period for which a subject having a disease (e.g. cancer) survives, without a significant worsening of the disease state. Progression free survival may be assessed as a period of time in which there is no progression of tumor growth and/or wherein the disease status of a patient is not determined to be a progressive disease. In some embodiments, progression free survival of a subject having cancer is assessed by evaluating tumor (lesion) size, tumor (lesion) number, clinical signs of progression, and/or metastasis.

As used herein, “progression free survival 2” (PFS2) is defined as time period from treatment randomization to the earlier date of assessment progression on the next anticancer therapy following study treatment or death by any cause. In some embodiments, determination of progression may be assessed by clinical and/or radiographic assessment.

The term “progression” of tumor growth or a “progressive disease” (PD) as used herein in reference to cancer status indicates an increase in the sum of the diameters of the target lesions (tumors). In some embodiments, progression of tumor growth refers to at least a 20% increase in the sum of diameters of target lesions, taking as reference the smallest sum on study (this includes the baseline sum if that is the smallest on study). In some embodiments, in addition to a relative increase of 20%, the sum of diameters of target lesions must also demonstrate an absolute increase of at least 5 mm. An appearance of one or more new lesions may also be factored into the determination of progression of tumor growth. Progression for the purposes of determining progression free survival may also be determined if at least one of the following criteria is met: 1) tumor assessment by CT/MRI unequivocally shows progressive disease according to RECIST 1.1 criteria; or 2) additional diagnostic tests (e.g., histology/cytology, ultrasound techniques, endoscopy, positron emission tomography) identify new lesions or determine existing lesions qualify for unequivocal progressive disease AND CA-125-progression according to Gynecologic Cancer Intergroup (GCIG)-criteria (see Rustin et al., “Definitions for Response and Progression in Ovarian Cancer Clinical Trials Incorporating RECIST 1.1 and CA 125 Agreed by the Gynecological Cancer Intergroup (GCIG)”, Int J Gynecol Cancer 2011; 21: 419-23, which is incorporated herein in its entirety); 3) definitive clinical signs and symptoms of PD unrelated to non-malignant or iatrogenic causes ([i] intractable cancer-related pain; [ii] malignant bowel obstruction/worsening dysfunction; or [iii] unequivocal symptomatic worsening of ascites or pleural effusion) AND CA-125-progression according to GCIG-criteria.

As used herein, the term “partial response” or “PR” refers to a decrease in tumor progression in a subject as indicated by a decrease in the sum of the diameters of the target lesions, taking as reference the baseline sum diameters. In some embodiments, PR refers to at least a 30% decrease in the sum of diameters or target lesions, taking as reference the baseline sum diameters. Exemplary methods for evaluating partial response are identified by RECIST guidelines. See E. A. Eisenhauer, et al., “New response evaluation criteria in solid tumors: Revised RECIST guideline (version 1.1.),” Eur. J. of Cancer, 45: 228-47 (2009).

As used herein, “stabilization” of tumor growth or a “stable disease” (SD) refers to neither sufficient shrinkage to qualify for PR nor sufficient increase to qualify for PD. In some embodiments, stabilization refers to a less than 30%, 25%, 20%, 15%, 10%, or 5% change (increase or decrease) in the sum of the diameters of the target lesions, taking as reference the baseline sum diameters. Exemplary methods for evaluating stabilization of tumor growth or a stable disease are identified by RECIST guidelines. See E. A. Eisenhauer, et al. “New response evaluation criteria in solid tumors: Revised RECIST guideline (version 1.1.),” Eur. J. of Cancer, 45: 228-47 (2009).

As used herein, the term “complete response” or “CR” is used to mean the disappearance of all or substantially all target lesions. In some embodiments, CR refers to an 80%, 85%, 90%, 91%, 92%, 93%, 94%, 95%, 96%, 97%, 98%, 99%, or 100% decrease in the sum of the diameters of the target lesions (i.e. loss of lesions), taking as reference the baseline sum diameters. In some embodiments, CR indicates that less than 10%, 9%, 8%, 7%, 6%, 5%, 4%, 3%, 2%, 1%, or less of the total lesion diameter remains after treatment. Exemplary methods for evaluating complete response are identified by RECIST guidelines. See E. A. Eisenhauer, et al. “New response evaluation criteria in solid tumors: Revised RECIST guideline (version 1.1.),” Eur. J. of Cancer, 45: 228-47 (2009).

As used herein, a “hazard ratio” (or “HR” when used in the context of niraparib treatment effect calculations, e.g. HR 0.38) is the expression of the hazard or chance of events occurring in the treatment arm as a ratio of the events occurring in the control arm. Hazard ratios may be determined by the Cox model, a regression method for survival data, which provides an estimate of the hazard ratio and its confidence interval. The hazard ratio is an estimate of the ratio of the hazard rate in the treated versus the control group. The hazard rate is the probability that if the event in question has not already occurred, it will occur in the next time interval, divided by the length of that interval. An assumption of proportional hazards regression is that the hazard ratio is constant over time.

In some embodiments, the present invention involves comparisons of results achieved for two or more agents, entities, situations, sets of conditions, populations, etc. As will be understood by those of skill in the art, such agents, entities, situations, sets of conditions, populations, etc. can be considered “comparable” to one another when they are not identical but are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Comparisons as described herein are often made to an appropriate “reference”. As used herein, the term “reference” refers to a standard or control relative to which a comparison is performed. For example, in some embodiments, an agent, animal, individual, population, sample, sequence, or value of interest is compared with a reference or control agent, animal, individual, population, sample, sequence, or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used here, the term “fasted state” refers to a state of a subject wherein food has not been consumed by the subject for a certain period of time. In some embodiments, a fasted state indicates that there is substantially no residual food in the stomach of the subject. In some embodiments, a fasted state refers to the state of the subject during the time from about 2- or more hours after food consumption up until about 30-minutes before the next food consumption. In some embodiments, the fasted state of a subject includes the time from about 2-hours after food consumption, 3-hours after food consumption, 3.5-hours after food consumption, 4-hours after food consumption, 6-hours after food consumption, 8-hours after food consumption, or 12-hours after food consumption, up until about 30-minutes before the next food consumption, or any time points between, end points inclusive.

As used here, the term “fed state” refers to a state of a subject wherein there is food in the stomach of the subject at the time of administration of a therapeutic agent (e.g., niraparib). In some embodiments, a fed state refers to the state of the subject during the time from the start of food consumption to about 2-hours after food consumption, such as during food consumption, immediately after food consumption, about 30-minutes after food consumption, about 1-hour after food consumption, about 1.5-hours after food consumption, about 2-hours after food consumption, or any time between any of the two numbers, end points inclusive. As used herein, food consumption refers to consuming a substantial amount of food, such as at least one third of a normal meal of a subject, either by volume or by total number of calories consumed.

As used herein, the term “polymorph” refers to a crystal structure of a compound. As used herein, the term “solvate” refers to a crystal form with either a stoichiometric or non-stoichiometric amount of solvent incorporated into the crystal structure. Similarly, the term “hydrate” refers to a crystal form with either a stoichiometric or non-stoichiometric amount of water incorporated into the crystal structure.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences 66: 1-19 (1977), incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for oral administration, for example, drenches (aqueous or non-aqueous solutions or suspensions), tablets, e.g., those targeted for buccal, sublingual, and systemic absorption, boluses, powders, granules, pastes for application to the tongue. A pharmaceutical composition can also refer to a medicament.

As used herein, the term “niraparib” means any of the free base compound ((3S)-3-[4-{7-(aminocarbonyl)-2H-indazol-2-yl}phenyl]piperidine), a salt form, including pharmaceutically acceptable salts, of (3S)-3-[4-{7-(aminocarbonyl)-2H-indazol-2-yl}phenyl]piperidine (e.g., (3S)-3-[4-{7-(aminocarbonyl)-2H-indazol-2-yl}phenyl]piperidine tosylate), or a solvated or hydrated form thereof (e.g., (3S)-3-[4-{7-(aminocarbonyl)-2H-indazol-2-yl}phenyl]piperidine tosylate monohydrate). In some embodiments, such forms may be individually referred to as “niraparib free base”, “niraparib tosylate”, and “niraparib tosylate monohydrate”, respectively. Unless otherwise specified, the term “niraparib” includes all forms of the compound (3S)-3-[4-{7-(aminocarbonyl)-2H-indazol-2-yl}phenyl]piperidine.

As used herein, the term “maintenance therapy” or “maintenance treatment” is a treatment that is given to prevent relapse of a disease. For example, a maintenance therapy may prevent or minimize growth of a cancer after it has been substantially reduced or eliminated following an initial therapy (cancer treatment). Maintenance therapy may be a continuous treatment where multiple doses are administered at spaced intervals such as every day, every other day, every week, every 2-weeks, every 3-weeks, every 4-weeks, or every 6-weeks. In some embodiments a maintenance therapy may continue for a predetermined length of time. In some embodiments, a maintenance therapy may continue until unacceptable toxicity occurs and/or disease progression occurs. In the course of maintenance treatment, treatment may be interrupted upon the occurrence of toxicity as indicated by an adverse event. If toxicity is appropriately resolved to baseline or grade 1 or less within 28-days, the patient may restart treatment, which may include a dose level reduction, if prophylaxis is not considered feasible.

As used herein, overall survival (“OS”) is defined as time from commencement of treatment to death from any cause. With respect to use as a clinical trial endpoint, it is defined as the time from randomization until death from any cause, and is measured in the intent to treat population.

As used herein, “objective response rate (“ORR”) is defined as the proportion of patients with tumor size reduction of a predefined amount and for a minimum period of time. Response duration is usually measured from the time of initial response until documented tumor progression. Generally, the ORR can be defined as the sum of partial responses plus complete responses.

As used herein, “time to first subsequent therapy” (TFST) is defined as the date of randomization in the current study to the start date of the first subsequent treatment regimen (e.g., anticancer therapy).

As used herein, “time to second subsequent therapy” (TSST) is defined as the date of randomization in the current study to the start date of the second subsequent treatment regimen (e.g., anticancer therapy).

As used herein, “chemotherapy-free interval” (CFI) is defined as the time from last dose of the last anticancer therapy (e.g., platinum-based chemotherapy) until the initiation of the next dose.

DNA Repair Pathways

Various pathways exist for DNA repair, including base excision repair (BER), direct repair (DR), double stranded break (DSB) repair, homologous recombination repair (HRR), mismatch repair (MMR), nucleotide excision repair (NER), and non-homologous end joining (NHEJ) repair; disruptions in these pathways can lead to the development and/or growth of cancer. See, e.g., Kelley et al., “Targeting DNA repair pathways for cancer treatment: what's new?”, Future Oncol., 10(7):1215-37 (2014).

Exemplary genes involved in DNA repair pathways are described in Table 1.

TABLE 1
DNA Repair Genes

| Gene Title | Gene Symbol |
|---|---|
| replication factor C (activator 1) 2, 40kDa | RFC2 |
| X-ray repair complementing defective repair in Chinese hamster | XRCC6 |
| cells 6 (Ku autoantigen, 70kDa) | |
| polymerase (DNA directed), delta 2, regulatory subunit 50kDa | POLD2 |
| proliferating cell nuclear antigen | PCNA |
| replication protein A1, 70kDa | RPA1 |
| replication protein A2, 32kDa | RPA2 |
| excision repair cross-complementing rodent repair deficiency, | ERCC3 |
| complementation group 3 (xeroderma pigmentosum group B | |
| complementing) | |
| uracil-DNA glycosylase | UNG |
| excision repair cross-complementing rodent repair deficiency, | ERCC5 |
| complementation group 5 (xeroderma pigmentosum, | |
| complementation group G (Cockayne syndrome)) | |
| mutL homolog 1, colon cancer, nonpolyposis type 2 (E. coli) | MLH1 |
| ligase I, DNA, ATP-dependent | LIG1 |
| mutS homolog 6 (E. coli) | MSH6 |
| polymerase (DNA-directed), delta 4 | POLD4 |
| replication factor C (activator 1) 5, 36.5kDa | RFC5 |
| damage-specific DNA binding protein 2, 48kDa///LIM | DDB2///LHX3 |
| homeobox 3 | |
| polymerase (DNA directed), delta 1, catalytic subunit 125kDa | POLD1 |
| Fanconi anemia, complementation group G | FANCG |
| polymerase (DNA directed), beta | POLB |
| X-ray repair complementing defective repair in Chinese hamster | XRCC1 |
| cells 1 | |
| N-methylpurine-DNA glycosylase | MPG |
| excision repair cross-complementing rodent repair deficiency, | |
| complementation group 1 (includes overlapping antisense | ERCC1 |
| sequence) | |
| thymine-DNA glycosylase | TDG |
| Fanconi anemia, complementation group A /// Fanconi anemia, | FANCA |
| complementation group A | |
| replication factor C (activator 1) 4, 37kDa | RFC4 |
| replication factor C (activator 1) 3, 38kDa | RFC3 |
| APEX nuclease (apurinic/apyrimidinic endonuclease) 2 | APEX2 |
| RAD1 homolog (S. pombe) | RAD1 |
| breast cancer 1, early onset | BRCA1 |
| exonuclease 1 | EXO1 |
| flap structure-specific endonuclease 1 | FEN1 |
| mutL homolog 3 (E. coli) | MLH3 |
| 0-6-methylguanine-DNA methyltransferase | MGMT |
| RAD51 homolog (RecA homolog, E. coli) (S. cerevisiae) | RAD51 |
| X-ray repair complementing defective repair in Chinese hamster | |
| cells 4 | XRCC4 |
| RecQ protein-like (DNA helicase Qi -like) | RECQL |
| excision repair cross-complementing rodent repair deficiency, | ERCC8 |
| complementation group 8 | |
| Fanconi anemia, complementation group C | FANCC |
| 8-oxoguanine DNA glycosylase | OGG1 |
| MRE11 meiotic recombination 11 homolog A (S. cerevisiae) | MRE11A |
| RAD52 homolog (S. cerevisiae) | RAD52 |
| Werner syndrome | WRN |
| xeroderma pigmentosum, complementation group A | XPA |
| Bloom syndrome | BLM |
| mutS homolog 3 (E. coli) | MSH3 |
| polymerase (DNA directed), epsilon 2 (p59 subunit) | POLE2 |
| RAD51 homolog C (S. cerevisiae) | RAD51C |
| ligase IV, DNA, ATP-dependent | LIG4 |
| excision repair cross-complementing rodent repair deficiency, | ERCC6 |
| complementation group 6 | |
| ligase III, DNA, ATP-dependent | LIG3 |
| RAD17 homolog (S. pombe) | RAD17 |
| X-ray repair complementing defective repair in Chinese hamster | |
| cells 2 | XRCC2 |
| mutY homolog (E. coli) | MUTYH |
| replication factor C (activator 1) 1, 145kDa///replication factor C | |
| (activator 1) 1, 145kDa | RFC1 |
| breast cancer 2, early onset | BRCA2 |
| RAD50 homolog (S. cerevisiae) | RAD50 |
| damage-specific DNA binding protein 1, 127kDa | DDB1 |
| X-ray repair complementing defective repair in Chinese hamster | XRCC5 |
| cells 5 (double-strand-break rejoining; Ku autoantigen, 80kDa) | |
| poly (ADP-ribose) polymerase family, member 1 | PARP1 |
| polymerase (DNA directed), epsilon 3 (p17 subunit) | POLE3 |
| xeroderma pigmentosum, complementation group C | XPC |
| mutS homolog 2, colon cancer, nonpolyposis type 1 (E. coli) | MSH2 |
| replication protein A3, 14kDa | RPA3 |
| methyl-CpG binding domain protein 4 | MBD4 |
| nth endonuclease III-like 1 (E. coli) | NTHL1 |
| PMS2 postmeiotic segregation increased 2 (S. cerevisiae)/// | PMS2///PMS2CL |
| PMS2-C terminal-like | |
| uracil-DNA glycosylase 2 | UNG2 |
| APEX nuclease (multifunctional DNA repair enzyme) 1 | APEX1 |
| excision repair cross-complementing rodent repair deficiency, | ERCC4 |
| complementation group 4 | |
| RecQ protein-like 5 | RECQL5 |
| mutS homolog 5 (E. coli) | MSH5 |
| polymerase (DNA-directed), delta 3, accessory subunit | POLD3 |
| excision repair cross-complementing rodent repair deficiency, | ERCC2 |
| complementation group 2 (xeroderma pigmentosum D) | |
| RecQ protein-like 4 | RECQL4 |
| PMS1 postmeiotic segregation increased 1 (S. cerevisiae) | PMS1 |
| zinc finger protein 276 homolog (mouse) | ZFP276 |
| polymerase (DNA directed), epsilon | POLE |
| X-ray repair complementing defective repair in Chinese hamster | XRCC3 |
| cells 3 | |
| nibrin | NBN |
| single-strand selective monofunctional uracil DNA glycosylase | SMUG1 |
| Fanconi anemia, complementation group F | FANCF |
| nei endonuclease VIII-like 1 (E. coli) | NEIL1 |
| Fanconi anemia, complementation group E | FANCE |
| Ataxia Telangiectasia Mutated | ATM |
| ATM and RAD3-related | ATR |
| BRCA1 associated protein-1 (ubiquitin carboxy-terminal | BAP1 |
| hydrolase) gene | |
| BRCA1 Associated RING Domain 1 (RING-Type E3 Ubiquitin | BARD1 |
| Transferase) gene | |
| BRCA1 Interacting Protein C-Terminal Helicase 1 gene | BRIP1 |
| Partner and localizer of BRCA2 gene | PALB2 |
| RAD51 Paralog B | RAD51B |
| RAD51 Paralog D | RAD51D |
| RAD54 Like | RAD54L |
| Human p53 gene | TP53 |
| Retinoblastoma gene | RB1 |

In one aspect, the invention features a method of treating cancer comprising: identifying a cancer patient having deficiency in at least one gene listed in Table 1 (e.g., RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, M1B14, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L, or combinations thereof); and administering a PARP inhibitor (e.g., niraparib) to the cancer patient. In embodiments, a deficiency is in two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more genes listed in Table 1.

In another aspect, the invention features a method of treating cancer comprising: administering a PARP inhibitor (e.g., niraparib) to a cancer patient identified to have deficiency in at least one gene listed in Table 1 (e.g., RFC2, XRCC6, POLD2, PCNA, RPA1, RPA2, ERCC3, UNG, ERCC5, MLH1, LIG1, MSH6, POLD4, RFC5, DDB2 ///LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L, or combinations thereof). In embodiments, a deficiency is in two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more genes listed in Table 1.

Poly(ADP-Ribose) Polymerases (PARPs)

For example, the poly(ADP-ribose) polymerase (PARP) family of proteins consists of over 15 different enzymes, which engage in a variety of cellular functions, including cell cycle regulation, transcription, and repair of DNA damage. PARP enzymes can cleave NAD+, releasing nicotinamide, and successively add ADP-ribose units to form ADP-ribose polymers. Accordingly, activation of PARP enzymes can lead to depletion of cellular NAD+ levels (e.g., PARPs as NAD+ consumers) and mediates cellular signaling through ADP-ribosylation of downstream targets. The role of PARP enzymes in DNA damage response (e.g. repair of DNA in response to genotoxic stress) has led to the compelling suggestion that PARP inhibitors may be useful anti-cancer agents.

PARP-1 is a zinc-finger DNA-binding enzyme that is activated by binding to DNA double or single strand breaks and is critical to the repair of single-strand DNA breaks through the base excision repair (BER) pathway. If such breaks persist unrepaired until DNA is replicated (which must precede cell division), then the replication itself can cause double strand breaks to form. Effective inhibition of PARP-1 leads to the accumulation of single-strand breaks, which ultimately results in double-strand breaks. Usually such double-strand breaks are repaired by homologous recombination (HR), but in cells with defective HR, PARP inhibition can result in chromosomal instability, cell cycle arrest, and subsequent apoptosis. DNA is damaged thousands of times during each cell cycle, and that damage must be repaired. When subjected to enough damage at one time, the altered gene can cause the death of the cells. Normal cells that don't replicate their DNA as often as cancer cells, and that lack any mutated BRCA1 or BRCA2 still have homologous repair operating, which allows them to survive the inhibition of PARP. PARP inhibitors function by blocking PARP enzyme activity, which prevents the repair of DNA damage and ultimately may cause cell death. They also are believed to function by localizing PARP proteins at sites of DNA damage, which has relevance to their anti-tumor activity. The trapped PARP protein-DNA complexes are highly toxic to cells because they block DNA replication.

PARP-2 contains a catalytic domain and is capable of catalyzing a poly(ADP-ribosyl)ation reaction. PARP-2 displays auto-modification properties similar to PARP-1. The protein is localized in the nucleus in vivo and may account for the residual poly(ADP-ribose) synthesis observed in PARP-1-deficient cells, treated with alkylating agents or hydrogen peroxide.

Studies have been directed to investigating the activity of PARP inhibitors, alone or in combination with other agents, as cancer therapeutics. PARP inhibitors may be particularly effective in treating cancers resulting from germ line or sporadic deficiency in the homologous recombination DNA repair pathway, such as BRCA-1, BRCA-2, and/or ATM deficient cancers. Additionally, simultaneous administration of genotoxic chemotherapy with PARP inhibition may enhance the killing effect of such chemotherapy by suppressing BER.

Pre-clinical ex vivo and in vivo experiments suggest that PARP inhibitors are selectively cytotoxic for tumors with homozygous inactivation of either the BRCA-1 or BRCA-2 genes, which are known to be important in the homologous recombination (HR) DNA repair pathway. The biological basis for the use of PARP-1 inhibitors as single agents in cancers with defects in HR is the requirement of PARP-1 and PARP-2 for base excision repair (BER) of the damaged DNA. Upon formation of single-strand DNA breaks, PARP-1 and PARP-2 bind at sites of lesions, become activated, and catalyze the addition of long polymers of ADP-ribose (PAR chains) on several proteins associated with chromatin, including histones, PARP itself, and various DNA repair proteins. This results in chromatin relaxation and fast recruitment of DNA repair factors that access and repair DNA breaks. Normal cells repair up to 10,000 DNA defects daily and single strand breaks are the most common form of DNA damage. Cells with defects in the BER pathway enter S phase with unrepaired single strand breaks. Pre-existing single strand breaks are converted to double strand breaks as the replication machinery passes through the break. Double strand breaks present during S phase are preferentially repaired by the error-free HR pathway. Cells unable to use HR (e.g., due to inactivation of genes required for HR, such as BRCA-1 or BRCA-2) accumulate stalled replication forks during S phase and may use error-prone non-homologous end joining (NHEJ) to repair damaged DNA. Both the inability to complete S phase (because of stalled replication forks) and error-prone repair by NHEJ, are thought to contribute to cell death.

PARP proteins are typically released from DNA once the DNA binding and repair process is underway. There is evidence to demonstrate that, when the proteins are bound to PARP inhibitors, they become trapped on DNA. The trapped PARP-DNA complexes are more toxic to cells than the unrepaired single-strand DNA breaks that accumulate in the absence of PARP activity. Therefore, without being limited as to theory, there are at least two mechanisms of action for PARP inhibitors: inhibition of repair and PARP trapping.

Homologous Recombination Repair (HRR) DNA Repair Pathway

Without wishing to be bound by theory, it is hypothesized that treatment with PARP inhibitors represents a novel opportunity to selectively kill a subset of cancer cells with deficiencies in DNA repair pathways, including certain deficiencies in the homologous recombination repair (HRR) pathway.

For example, a tumor arising in a patient with a germline BRCA mutation has a defective homologous recombination DNA repair pathway and would be increasingly dependent on BER, a pathway blocked by PARP inhibitors, for maintenance of genomic integrity. Non-BRCA deficiencies in homologous recombination DNA repair genes could also enhance tumor cell sensitivity to PARP inhibitors. This concept of inducing death by use of PARP inhibitors to block one DNA repair pathway in tumors with pre-existing deficiencies in a complementary DNA repair pathways is called synthetic lethality: the simultaneous inhibition of two pathways leads to cell death, whereas blocking either pathway alone is not lethal.

Cells unable to use HRR (e.g., due to inactivation of genes required for HRR, such as BRCA-1 or BRCA-2 or such as non-BRCA1/2 HRR genes such as any of ATM, ATR, BAP1, BARD1, BLM, BRIP1, MRE11A, NBN, PALB2, RAD51, RAD51B, RAD51C, RAD51D, RAD52, RAD54L, and XRCC2, and combinations thereof) accumulate stalled replication forks during S phase and may use error-prone non-homologous end joining (NHEJ) to repair damaged DNA. Both the inability to complete S phase (because of stalled replication forks) and error-prone repair by NHEJ, are thought to contribute to cell death.

Pre-clinical ex vivo and in vivo experiments suggest that PARP inhibitors are indeed selectively cytotoxic for tumors with homozygous inactivation of either the BRCA-1 or BRCA-2 genes, which are known to be important in the homologous recombination (HRR) DNA repair pathway. In particular, the inability of HRR to correct double-stranded breaks has been observed in tumors with mutations in BRCA-1 and BRCA-2, as these genes code for proteins essential for normal HR function. Germline mutations of BRCA-1 and BRCA-2 genes are found in a majority of patients with an inherited breast or ovarian cancer. Inactivation of BRCA-1 and BRCA-2 gene by other mechanisms, including somatic BRCA-1/2 mutations and/or gene silencing by promoter hypermethylation, occurs in a significant portion of several sporadic cancers. In particular, for ovarian cancer, somatic BRCA-1 or BRCA-2 mutations are found in 10%-15% of all epithelial ovarian carcinomas (EOCs), and strongly reduced expression of BRCA-1 has been observed in a significant portion of sporadic ovarian cancers. Collectively, up to 40%-60% of ovarian cancers might be responsive to PARP inhibitors as a consequence of defects in the BRCA-HRR pathway, indicating a great potential for this approach in the therapy of ovarian cancer. Thus, encouraging preclinical results for PARP inhibitors in the treatment of BRCA-mutated tumor cells provided strong rationale for the clinical testing of these agents in patient populations most likely to carry these mutations, such as those with breast or ovarian cancer.

HRR, however, is a complex pathway, and genes other than BRCA-1 and BRCA-2 are required either to sense or repair DNA double strand breaks via the HRR pathway. PARP inhibitors are also selectively cytotoxic for cancer cells with deficiencies in DNA repair-proteins other than BRCA-1 and BRCA-2. In particular, the present invention shows that deficiencies in non-BRCA1/2 HRR genes such as ATM, ATR, BAP1, BARD1, BLM, BRIP1, MRE11A, NBN, PALB2, RAD51, RAD51B, RAD51C, RAD51D, RAD52, RAD54L, and XRCC2 can result in responsiveness to treatment with PARP inhibitors (e.g., treatment with niraparib).

Non-BRCA HRR Deficiencies

The present invention is based in part on the discovery that PARP inhibitors (e.g., niraparib) can be used to treat cancers in patients identified to have non-BRCA deficiencies in the HRR pathway (e.g., a gene such as any of ATM, ATR, BAP1, BARD1, BLM, BRIP1, MRE11A, NBN, PALB2, RAD51, RAD51B, RAD51C, RAD51D, RAD52, RAD54L, and XRCC2, and any combinations thereof) in the presence or absence of deficiencies in BRCA1 and/or BRCA2.

In embodiments, the invention features a method of treating cancer, where the method comprises: identifying a cancer patient having deficiency in at least one gene involved in the homologous recombination repair (HRR) pathway, wherein the at least one gene involved in the HRR pathway is not BRCA1 or BRCA2; and administering a PARP inhibitor (e.g., niraparib) to the cancer patient.

In embodiments, the invention features a method of treating cancer, where the method comprises administering a PARP inhibitor (e.g., niraparib) to a cancer patient identified to have deficiency in at least one gene involved in the homologous recombination repair (HRR) pathway, wherein the at least one gene involved in the HRR pathway is not BRCA1 or BRCA2.

As shown herein in Table 1, there are a number of genes involved in the various DNA repair pathways. In some embodiments, cancer patients have HRR deficiencies due at least to one of the genes listed in Table 1. In embodiments, cancer patients having HRR deficiencies due to at least one of the sixteen genes listed in Table 2 benefit from administration of a PARP inhibitor (e.g., niraparib).

TABLE 2
Non-BRCA1/2 HRR Pathway Genes
HRR Pathway Genes

| | | |
|---|---|---|
| ATM | MRE11A | RAD51C |
| ATR | NBN | RAD51D |
| BAP1 | PALB2 | RAD52 |
| BARD1 | RAD51 | RAD54L |
| BLM | RAD51B | XRCC2 |
| BRIP1 | TP53 | RB1 |

In embodiments, a patient has a deficiency in a gene panel involved in the HRR pathway comprising TP53 and/or RB1. In embodiments, a patient has a deficiency in one or more of ATM, MRE11A, RAD51C, ATR, NBN, RAD51D, BAP1, PALB2, RAD52, BARD1, RAD51, RAD54L, BLM, RAD51B, XRCC2, BRIP1, TP53, and/or RB1. In embodiments, a patient has a deficiency in at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, or at least eighteen of ATM, MRE11A, RAD51C, ATR, NBN, RAD51D, BAP1, PALB2, RAD52, BARD1, RAD51, RAD54L, BLM, RAD51B, XRCC2, BRIP1, TP53, and/or RB1.

In embodiments, a patient has a deficiency in at least one, at least two, at least three, at least four, at least five, at least six, at least seven, at least eight, at least nine, at least ten, at least eleven, at least twelve, at least thirteen, at least fourteen, at least fifteen, or at least sixteen genes involved in the HRR pathway and which are not BRCA1 or BRCA2 (e.g., at least one of the genes of Table 2, and any combinations thereof). In embodiments, the at least one, at least two, at least three, at least four, at least five, at least six, at least seven, at least eight, at least nine, at least ten, at least eleven, at least twelve, at least thirteen, at least fourteen, or at least fifteen genes involved in the HRR pathway are selected from the genes of Table 2, and any combinations thereof. In embodiments, a patient has a deficiency in each of the genes of Table 2.

In embodiments, at least one deficiency in the HRR pathway is a mono-allelic mutation of a gene that is not BRCA1 or BRCA2 (e.g., any of the genes of Table 2, and combinations thereof). In embodiments, at least one, at least two, at least three, at least four, at least five, at least six, at least seven, at least eight, at least nine, at least ten, at least eleven, at least twelve, at least thirteen, at least fourteen, at least fifteen, or at least sixteen of the genes described in Table 2 independently have a mono-allelic mutation.

In embodiments, at least one deficiency in the HRR pathway is a bi-allelic mutation of a gene that is not BRCA1 or BRCA2 (e.g., any of the genes of Table 2, and combinations thereof). In embodiments, at least one, at least two, at least three, at least four, at least five, at least six, at least seven, at least eight, at least nine, at least ten, at least eleven, at least twelve, at least thirteen, at least fourteen, at least fifteen, or at least sixteen of the genes described in Table 2 independently have a bi-allelic mutation.

In embodiments, at least one, at least two, at least three, at least four, at least five, at least six, at least seven, at least eight, at least nine, at least ten, at least eleven, at least twelve, at least thirteen, at least fourteen, at least fifteen, or at least sixteen of the genes described in Table 2 independently have a mono-allelic or a bi-allelic mutation.

In embodiments, a mono-allelic mutation is independently a germline mutation. In embodiments, a mono-allelic mutation is independently a sporadic mutation.

In embodiments, a bi-allelic mutation is independently a germline mutation. In embodiments, a bi-allelic mutation is independently a sporadic mutation.

In embodiments, a patient has an identified deficiency in BAP1. In embodiments, a patient has an identified deficiency in XRCC2. In embodiments, a patient has an identified deficiency in ATM. In embodiments, a patient has an identified deficiency in ATR. In embodiments, a patient has an identified deficiency in BARD1. In embodiments, a patient has an identified deficiency in BLM. In embodiments, a patient has an identified deficiency in BRIP1. In embodiments, a patient has an identified deficiency in MRE11A. In embodiments, a patient has an identified deficiency in NBN. In embodiments, a patient has an identified deficiency in PALB2. In embodiments, a patient has an identified deficiency in RAD51. In embodiments, a patient has an identified deficiency in RAD51B. In embodiments, a patient has an identified deficiency in RAD51C. In embodiments, a patient has an identified deficiency in RAD51D. In embodiments, a patient has an identified deficiency in RAD52. In embodiments, a patient has an identified deficiency in RAD54L.

In embodiments, a patient has an identified deficiency in one or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in one of the genes selected from the group consisting ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in two or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in two of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in three or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in three of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in four or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in four of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in five or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in five of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in six or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in six of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in seven or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in seven of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in eight or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in eight of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in nine or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in nine of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in ten or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in ten of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in eleven or more of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, a patient has an identified deficiency in eleven of the genes selected from the group consisting of ATM, ATR, BARD1, BRIP1, MRE11A, NBN, PALB2, RAD51, RAD51B, RAD51C, RAD51D, and RAD54L. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in one or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in one of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in two or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in two of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in three or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in three of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in four or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in four of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in five or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in five of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in six or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in six of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in seven or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in seven of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in eight or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in eight of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in nine or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in nine of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in ten or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in ten of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in eleven or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in eleven of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in twelve or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in twelve of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in thirteen or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in thirteen of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in fourteen or more of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in fourteen of the genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in two or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in two of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in three or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in three of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in four or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in four of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in five or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in five of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in six or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in six of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in seven or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in seven of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in eight or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in eight of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in nine or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in nine of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in ten or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in ten of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in eleven or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in eleven of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in twelve or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in twelve of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in thirteen or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in thirteen of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in fourteen or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in fourteen of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in fifteen or more of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, a patient has an identified deficiency in fifteen of the genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient has an identified deficiency in each of ATM, ATR, BAP1, BARD1, BLM, BRIP1, MRE11A, NBN, PALB2, RAD51, RAD51B, RAD51C, RAD51D, RAD52, RAD54L, and XRCC2. Alternatively, or in addition to, a patient has an identified deficiency in one or more of the genes TP3 and/or RB1. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

In embodiments, a patient having a deficiency in a non-BRCA1/2 HRR pathway gene as described herein (e.g., at least one of the genes of Table 2, and any combinations thereof) also has a deficiency in one or more of the genes listed in Table 1 (e.g., RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, or FANCE, or combinations thereof). Alternatively, or in addition to, a patient has an identified deficiency in one or more of the genes TP3 and/or RB1. In embodiments, a deficiency is in two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more genes listed in Table 1. In embodiments, a deficiency is an identified deficiency. In embodiments, an identified deficiency is a germline mutation. In embodiments, at least one identified deficiency is a germline mutation. In embodiments, an identified deficiency is a sporadic mutation. In embodiments, at least one identified deficiency is a sporadic mutation. In embodiments, an identified deficiency is independently a mono-allelic mutation. In embodiments, at least one identified deficiency is a mono-allelic mutation. In embodiments, an identified deficiency is independently a bi-allelic mutation. In embodiments, at least one identified deficiency is a bi-allelic mutation. In embodiments, each identified deficiency is a mono-allelic mutation. In embodiments, each identified deficiency is a bi-allelic mutation.

BRCA1 and BRCA2 HRR Deficiencies

BRCA 1 and 2 were initially identified as tumor suppressor genes that were associated with increased incidence of certain malignancies when defective. In some embodiments, a cancer has one or more of germline BRCA mutation, sporadic BRCA mutation and BRCA promoter hypermethylation. In some embodiments, a cancer has a combination of two or more of germline BRCA mutation, sporadic BRCA mutation and BRCA promoter hypermethylation. Germline mutations of BRCA-1 and BRCA-2 genes are found in a majority of patients with an inherited breast or ovarian cancer. Inactivation of BRCA-1 or BRCA-2 gene by other mechanisms, including somatic BRCA-1/2 mutations and/or gene silencing by promoter hypermethylation, occurs in a significant portion of several sporadic cancers. In particular, for ovarian cancer, somatic BRCA-1 or BRCA-2 mutations are found in 10%-15% of all epithelial ovarian carcinomas (EOCs), and strongly reduced expression of BRCA-1 has been observed in a significant portion of sporadic ovarian cancers.

In some embodiments, a subject to be treated by methods of the present disclosure is characterized by a “positive BRCA status”, “BRCA+”, or “BRCA-mutant”. In some embodiments, a patient with a “positive BRCA status” refers to a patient from whom a sample has been determined to have a reduced expression of BRCA1 and/or BRCA2.

In some embodiments, a subject to be treated by methods of the present disclosure is characterized by a “negative BRCA status”, “BRCA-”, or “BRCA-wild type”. In some embodiments a negative BRCA status refers to a patient from whom a sample has been

A cancer patient who has a deficiency in a non-BRCA1/2 gene involved in the HRR pathway as described herein (e.g., an identified deficiency in at least one, at least two, at least three, at least four, at least five, at least six, at least seven, at least eight, at least nine, at least ten, at least eleven, at least twelve, at least thirteen, at least fourteen, at least fifteen of the genes of Table 2, and any combinations thereof) can benefit from methods described herein in the presence or absence of deficiencies in BRCA1 and/or BRCA2. In embodiments, a BRCA1/2 deficiency is a germline mutation (gBRCA^{mut}). In embodiments, a BRCA1/2 deficiency is a sporadic mutation (sBRCA^{mut}). In some embodiments, a patient the population of subjects exhibits non-mutated BRCA1/2 (BRCA^{wt}).

In embodiments, a patient having a deficiency in at least one non-BRCA1 or non-BRCA2 gene involved in the HRR pathway as described herein (e.g., an identified deficiency in at least one, at least two, at least three, at least four, at least five, at least six, at least seven, at least eight, at least nine, at least ten, at least eleven, at least twelve, at least thirteen, at least fourteen, at least fifteen of the genes of Table 2, and any combinations thereof) does not have any germline mutations in BRCA1 or in BRCA2.

In embodiments, a patient having a deficiency in at least one non-BRCA1 or non-BRCA2 gene involved in the HRR pathway as described herein (e.g., an identified deficiency in at least one, at least two, at least three, at least four, at least five, at least six, at least seven, at least eight, at least nine, at least ten, at least eleven, at least twelve, at least thirteen, at least fourteen, at least fifteen of the genes of Table 2, and any combinations thereof) also has at least one germline mutation in BRCA1 and/or in BRCA2. In embodiments, a patient has at least one germline mutation in BRCA1. In embodiments, a patient has at least one germline mutation in BRCA2. In embodiments, a patient has at least one germline mutation in each of BRCA1 and BRCA2.

In embodiments, a patient having a deficiency in at least one non-BRCA1 or non-BRCA2 gene involved in the HRR pathway as described herein (e.g., an identified deficiency in at least one, at least two, at least three, at least four, at least five, at least six, at least seven, at least eight, at least nine, at least ten, at least eleven, at least twelve, at least thirteen, at least fourteen, at least fifteen of the genes of Table 2, and any combinations thereof) does not have any sporadic mutations in BRCA1 or in BRCA2.

In embodiments, a patient having a deficiency in at least one non-BRCA1 or non-BRCA2 gene involved in the HRR pathway as described herein (e.g., an identified deficiency in at least one, at least two, at least three, at least four, at least five, at least six, at least seven, at least eight, at least nine, at least ten, at least eleven, at least twelve, at least thirteen, at least fourteen, at least fifteen of the genes of Table 2, and any combinations thereof) also has at least one sporadic mutation in BRCA1 and/or in BRCA2. In embodiments, a patient has at least one sporadic mutation in BRCA1. In embodiments, a patient has at least one sporadic mutation in BRCA2. In embodiments, a patient has at least one sporadic mutation in each of BRCA1 and BRCA2.

In embodiments, an identified deficiency is a bi-allelic mutation in ATM, BAP1, and BRCA genes.

Identification of HRR Deficiencies

Deficiencies in the HRR pathway (e.g., a deficiency in at least one non-BRCA1 or non-BRCA2 gene involved in the HRR pathway and/or a deficiency in BRCA1 and/or BRCA2) can be identified using methods known in the art. For example, the identification of a deficiency in the HRR pathway can include determinations made by a standardized laboratory test, such as and also including those tests approved by a relevant regulatory authority.

In embodiments, a deficiency in a gene involved in the HRR pathway is identified using a pre-specified gene panel. In embodiments, a pre-specified gene panel includes a gene listed in Table 1 or Table 2, or any combinations thereof. In embodiments, a pre-specified gene panel includes one or more genes listed in Table 1 (e.g., RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, or RAD54L). In embodiments, a pre-specified gene panel comprises two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more genes listed in Table 1.

In embodiments, a deficiency in a gene involved in the HRR pathway is identified using a pre-specified HRR gene panel.

In embodiments, a pre-specified HRR gene panel comprises BAP1. In embodiments, a pre-specified HRR gene panel comprises XRCC2. In embodiments, a pre-specified HRR gene panel comprises ATM. In embodiments, a pre-specified HRR gene panel comprises ATR. In embodiments, a pre-specified HRR gene panel comprises BARD1. In embodiments, a pre-specified HRR gene panel comprises BLM. In embodiments, a pre-specified HRR gene panel comprises BRIP1. In embodiments, a pre-specified HRR gene panel comprises MRE11A. In embodiments, a pre-specified HRR gene panel comprises NBN. In embodiments, a pre-specified HRR gene panel comprises PALB2. In embodiments, a pre-specified HRR gene panel comprises RAD51. In embodiments, a pre-specified HRR gene panel comprises RAD51B. In embodiments, a pre-specified HRR gene panel comprises RAD51C. In embodiments, a pre-specified HRR gene panel comprises RAD51D. In embodiments, a pre-specified HRR gene panel comprises RAD52. In embodiments, a pre-specified HRR gene panel comprises RAD54L.

In embodiments, a pre-specified HRR gene panel comprises one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L. In embodiments, a pre-specified HRR gene panel comprises each of ATM, ATR, BARD1, BRIP1, MRE11A, NBN, PALB2, RAD51, RAD51B, RAD51C, RAD51D, and RAD54L. In embodiments, a pre-specified HRR gene panel comprises each of ATM, ATR, BARD1, BRIP1, MRE11A, NBN, PALB2, RAD51, RAD51B, RAD51C, RAD51D, and RAD54L and further comprises BRCA1 and/or BRCA2. In embodiments, a pre-specified HRR gene panel comprises each of ATM, ATR, BARD1, BRIP1, MRE11A, NBN, PALB2, RAD51, RAD51B, RAD51C, RAD51D, RAD54L, BRCA1, and BRCA2. In embodiments, a pre-specified HRR gene panel further comprises at least one of the genes described in Table 1 (e.g., RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, or FANCE).

In embodiments, a pre-specified HRR gene panel comprises one or more, two or more, three or more, four or more, five or more, seven or more, eight or more, nine or more, ten or more, eleven or more, twelve or more, thirteen or more, fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2. In embodiments, a pre-specified HRR gene panel comprises each of ATM, ATR, BAP1, BARD1, BRIP1, MRE11A, NBN, PALB2, RAD51, RAD51B, RAD51C, RAD51D, RAD52, RAD54L, and XRCC2. In embodiments, a pre-specified HRR gene panel comprises each of ATM, ATR, BAP1, BARD1, BRIP1, MRE11A, NBN, PALB2, RAD51, RAD51B, RAD51C, RAD51D, RAD52, RAD54L, and XRCC2, and further comprises BRCA1 and/or BRCA2. In embodiments, a pre-specified HRR gene panel comprises each of ATM, ATR, BAP1, BARD1, BRIP1, MRE11A, NBN, PALB2, RAD51, RAD51B, RAD51C, RAD51D, RAD52, RAD54L, XRCC2, BRCA1, and BRCA2. In embodiments, a pre-specified HRR gene panel further comprises at least one of the genes described in Table 1 (e.g., RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, or FANCE).

In embodiments, a pre-specified HRR gene panel comprises one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a pre-specified HRR gene panel comprises ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a pre-specified HRR gene panel comprises each of ATM, ATR, BAP1, BARD1, BLM, BRIP1, MRE11A, NBN, PALB2, RAD51, RAD51B, RAD51C, RAD51D, RAD52, RAD54L, and XRCC2. In embodiments, a pre-specified HRR gene panel comprises each of ATM, ATR, BAP1, BARD1, BLM, BRIP1, MRE11A, NBN, PALB2, RAD51, RAD51B, RAD51C, RAD51D, RAD52, RAD54L, and XRCC2, and further comprises BRCA1 and/or BRCA2. In embodiments, a pre-specified HRR gene panel comprises each of ATM, ATR, BAP1, BARD1, BLM, BRIP1, MRE11A, NBN, PALB2, RAD51, RAD51B, RAD51C, RAD51D, RAD52, RAD54L, and XRCC2, BRCA1, and BRCA2. In embodiments, a pre-specified HRR gene panel further comprises at least one of the genes described in Table 1 (e.g., RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, XRCC4, RECQL, ERCC8, FANCC, OGG1, WRN, XPA, MSH3, POLE2, LIG4, ERCC6, LIG3, RAD17, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, SMUG1, FANCF, NEIL1, or FANCE).

In embodiments, administration of a PARP inhibitor (e.g., niraparib) occurs independent of the BRCA status.

In embodiments, a cancer patient's BRCA status is not determined prior to administration of a PARP inhibitor (e.g., niraparib). In embodiments, administration of a PARP inhibitor (e.g., niraparib) occurs in the absence of determining the BRCA status.

In embodiments, a cancer patient's BRCA status is determined prior to administration of a PARP inhibitor (e.g., niraparib). In embodiments, a cancer patient's BRCA status is determined following initial administration of a PARP inhibitor (e.g., niraparib).

A cancer patient's BRCA status can be determined according to methods known in the art. For example, the identification of a deficiency in the HRR pathway can include determinations made by a standardized laboratory test, such as and also including those tests approved by a relevant regulatory authority. In embodiments, a deficiency in BRCA1/2 can be determined a pre-specified gene panel comprising BRCA1 and/or BRCA2.

In embodiments, a pre-specified gene panel comprises: at least one of ATM, ATR, BARD1, BRIP1, MRE11A, NBN, PALB2, RAD51, RAD51B, RAD51C, RAD51D, and RAD54L and any combinations thereof, and at least one of BRCA1 and BRCA2. In embodiments, a pre-specified gene panel comprises: each of ATM, ATR, BARD1, BRIP1, MRE11A, NBN, PALB2, RAD51, RAD51B, RAD51C, RAD51D, and RAD54L; and at least one of BRCA1 and BRCA2. In embodiments, a pre-specified gene panel comprises ATM, ATR, BARD1, BRIP1, MRE11A, NBN, PALB2, RAD51, RAD51B, RAD51C, RAD51D, RAD54L, BRCA1, and BRCA2.

In embodiments, a pre-specified gene panel comprises: at least one of ATM, ATR, BAP1, BARD1, BRIP1, MRE11A, NBN, PALB2, RAD51, RAD51B, RAD51C, RAD51D, RAD52, RAD54L, and XRCC2, and any combinations thereof, and at least one of BRCA1 and BRCA2. In embodiments, a pre-specified gene panel comprises: each of ATM, ATR, BAP1, BARD1, BRIP1, MRE11A, NBN, PALB2, RAD51, RAD51B, RAD51C, RAD51D, RAD52, RAD54L, and XRCC2 and at least one of BRCA1 and BRCA2. In embodiments, a pre-specified gene panel comprises ATM, ATR, BAP1, BARD1, BRIP1, MRE11A, NBN, PALB2, RAD51, RAD51B, RAD51C, RAD51D, RAD52, RAD54L, XRCC2, BRCA1, and BRCA2.

In embodiments, a pre-specified gene panel comprises: at least one of ATM, ATR, BAP1, BARD1, BLM, BRIP1, MRE11A, NBN, PALB2, RAD51, RAD51B, RAD51C, RAD51D, RAD52, RAD54L, and XRCC2, and any combinations thereof, and at least one of BRCA1 and BRCA2. In embodiments, a pre-specified gene panel comprises: each of ATM, ATR, BAP1, BARD1, BLM, BRIP1, MRE11A, NBN, PALB2, RAD51, RAD51B, RAD51C, RAD51D, RAD52, RAD54L, and XRCC2; and at least one of BRCA1 and BRCA2. In embodiments, a pre-specified gene panel comprises ATM, ATR, BAP1, BARD1, BLM, BRIP1, MRE11A, NBN, PALB2, RAD51, RAD51B, RAD51C, RAD51D, RAD52, RAD54L, XRCC2, BRCA1, and BRCA2.

A gene deficiency (e.g., a deficiency in any of the genes listed in Table 1 or Table 2) can be identified by analyzing cancer cells or non-cancer cells; analyzing cell-free DNA; using sequencing methods; using PCR; or using an immunohistochemistry assay.

In embodiments, any HRR deficiency described herein (e.g., a deficiency in at least one of the genes in Table 2, and combinations thereof, and optionally in further combination with BRCA1 and/or BRCA2) is identified by analyzing cancer cells

In embodiments, any HRR deficiency described herein (e.g., a deficiency in at least one of the genes in Table 2, and combinations thereof, and optionally in further combination with BRCA1 and/or BRCA2) is identified by analyzing non-cancer cells.

In embodiments, cells (e.g., non-cancer cells) are obtained from one or more body fluids. In embodiments, cells (e.g., non-cancer cells) are obtained from blood (e.g., whole blood and/or plasma). In embodiments, cells (e.g., non-cancer cells) are obtained from saliva, urine, and/or cerebrospinal fluid. In embodiments, cells (e.g., non-cancer cells) are obtained from one or more tissue samples.

In embodiments, any HRR deficiency described herein (e.g., a deficiency in at least one of the genes in Table 2, and combinations thereof, and/or a deficiency in BRCA1 and/or BRCA2) is identified by analyzing cell-free DNA.

In embodiments, any HRR deficiency described herein (e.g., a deficiency in at least one of the genes in Table 2, and combinations thereof, and/or a deficiency in BRCA1 and/or BRCA2) is identified by sequencing.

In embodiments, any HRR deficiency described herein (e.g., a deficiency in at least one of the genes in Table 2, and combinations thereof, and/or a deficiency in BRCA1 and/or BRCA2) is identified by PCR.

In embodiments, any HRR deficiency described herein (e.g., a deficiency in at least one of the genes in Table 2, and combinations thereof, and/or a deficiency in BRCA1 and/or BRCA2) is identified by an immunohistochemistry assay.

PARP Inhibitors

The present invention is based in part on the discovery that PARP inhibitors can be used to treat cancers in patients having an identified deficiency in at least one gene involved in the homologous recombination repair (HRR) pathway, where the at least one gene involved in the HRR pathway is not BRCA1 or BRCA2.

In embodiments, a PARP inhibitor inhibits PARP-1 and/or PARP-2. In some embodiments, the agent is a small molecule, a nucleic acid, a polypeptide (e.g., an antibody), a carbohydrate, a lipid, a metal, or a toxin. In related embodiments, the agent is ABT-767, AZD 2461, BGB-290, BGP 15, CEP 8983, CEP 9722, DR 2313, E7016, E7449, fluzoparib (SHR 3162), IMP 4297, INO1001, JPI 289, JPI 547, monoclonal antibody B3-LysPE40 conjugate, MP 124, niraparib (ZEJULA) (MK-4827), NU 1025, NU 1064, NU 1076, NU1085, olaparib (AZD2281), ONO2231, PD 128763, R 503, R554, rucaparib (RUBRACA) (AG-014699, PF-01367338), SBP 101, SC 101914, simmiparib, talazoparib (BMN-673), veliparib (ABT-888), WW 46, 2-(4-(trifluoromethyl)phenyl)-7,8-dihydro-5H-thiopyrano[4,3-d]pyrimidin-4-ol, and salts or derivatives thereof. In some related embodiments, the agent is niraparib, olaparib, rucaparib, talazoparib, veliparib, or salts or derivatives thereof. In certain embodiments, the agent is niraparib or a salt or derivative thereof. In certain embodiments, the agent is olaparib or a salt or derivative thereof. In certain embodiments, the agent is rucaparib or a salt or derivative thereof. In certain embodiments, the agent is talazoparib or a salt or derivative thereof. In certain embodiments, the agent is veliparib or a salt or derivative thereof.

Niraparib

Niraparib, (3S)-3-[4-{7-(aminocarbonyl)-2H-indazol-2-yl}phenyl]piperidine, is an orally available, potent, poly (adenosine diphosphate [ADP]-ribose) polymerase (PARP)-1 and -2 inhibitor. See WO 2008/084261 (published on Jul. 17, 2008), WO 2009/087381 (published Jul. 16, 2009), and PCT/US17/40039 (filed Jun. 29, 2017), the entirety of each of which is hereby incorporated by reference. Niraparib can be prepared according to Scheme 1 of WO 2008/084261.

In some embodiments, niraparib can be prepared as a pharmaceutically acceptable salt. One of skill in the art will appreciate that such salt forms can exist as solvated or hydrated polymorphic forms. In some embodiments, niraparib is prepared in the form of a hydrate.

In certain embodiments, niraparib is prepared in the form of a tosylate salt. In some embodiments, niraparib is prepared in the form of a tosylate monohydrate. The molecular structure of the tosylate monohydrate salt of niraparib is shown below:

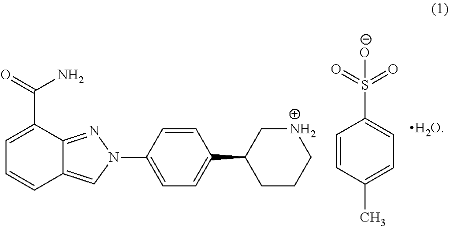

Niraparib is a potent and selective PARP-1 and PARP-2 inhibitor with inhibitory concentration at 50% of control (IC₅₀)=3.8 and 2.1 nM, respectively, and is at least 100-fold selective over other PARP-family members. Niraparib inhibits PARP activity, stimulated as a result of DNA damage caused by addition of hydrogen peroxide, in various cell lines with an IC₅₀ and an inhibitory concentration at 90% of control (IC₉₀) of about 4 and 50 nM, respectively.

Niraparib demonstrates selective anti-proliferative activity for cancer cell lines that have been silenced for BRCA-1 or BRCA-2, or carry BRCA-1 or BRCA-2 mutations compared to their wild type counterparts. The antiproliferative activity of niraparib on BRCA-defective cells is a consequence of a cell cycle arrest in G2/M followed by apoptosis. Niraparib can also be selectively cytotoxic for selected Ewing's sarcoma, acute lymphocytic leukemia (ALL), non-small cell lung cancer (NSCLC), and small cell lung cancer (SCLC) cell lines, as well as for tumor cell lines carrying homozygous inactivation of the ATM gene. Niraparib demonstrates weak activity on normal human cells. In vivo studies demonstrated strong antitumor activity with BRCA-1 mutant breast cancer (MDA-MB-436), BRCA-2 mutant pancreatic cancer (CAPAN-1), ATM-mutant mantle cell lymphoma (GRANTA-519), serous ovarian cancer (OVCAR3), colorectal cancer (HT29 and DLD-1), patient derived Ewing's sarcoma, and TNBC xenograft models in mice.

Olaparib

Olaparib acts as an inhibitor of the enzyme poly ADP ribose polymerase (PARP), and is termed a PARP inhibitor. The chemical name is 4-[(3-{[4-(cyclopropylcarbonyl)piperazin-1-yl]carbonyl}-4-fluorophenyl)methyl]phthalazin-1(2H)-one. Clinical trials of olaparib were initiated in breast, ovarian and colorectal cancer. Preliminary activity was seen in ovarian cancer, with 7 responses in 17 patients with BRCA1 or BRCA2 mutations and 11 responses in the 46 who did not have these mutations. However, an interim analysis of a phase II study that looked at using olaparib to maintain progression free survival or response after success with platinum-based chemotherapy indicated that a reported progression-free survival benefit was unlikely to translate into an overall survival benefit for the intent to treat populations. However, planned analysis of the subset of patients who had BRCA mutations found a clear advantage with olaparib (Ledermann et al., “Olaparib Maintenance Therapy in Platinum-Sensitive Relapsed Ovarian Cancer”, New England Journal of Medicine, 366:1382-92 (2012); Ledermann et al., “Olaparib maintenance therapy in patients with platinum-sensitive relapsed serous ovarian cancer: a preplanned retrospective analysis of outcomes by BRCA status in a radomised phase 2 trial”, Lancet Oncol. 15(8): 852-61 (2014)). Olaparib is approved as monotherapy, at a recommended dose of 400 mg taken twice per day, in germline BRCA mutated (gBRCAmut) advanced ovarian cancer that has received three or more prior lines of chemotherapy. BRCA1/2 mutations may be genetically predisposed to development of some forms of cancer, and may be resistant to other forms of cancer treatment. However, these cancers sometimes have a unique vulnerability, as the cancer cells have increased reliance on PARP to repair their DNA and enable them to continue dividing. This means that drugs which selectively inhibit PARP may be of benefit if the cancers are susceptible to this treatment. Thus, the olaparib clinical data demonstrated that PARP inhibitors would not be beneficial to prolong progression free survival in the treatment of cancer characterized by the absence of mutations in BRCA1 or BRCA2.

Rucaparib

Similarly, rucaparib acts as an inhibitor of the enzyme poly ADP ribose polymerase (PARP), and is also termed a PARP inhibitor. The chemical name is 8-fluoro-2-{4-[(methylamino)methyl]phenyl}-1,3,4,5-tetrahydro-6H-azepino[5,4,3-cd]indol-6-one ((1S,4R)-7,7-dimethyl-2-oxobicyclo[2.2.1]hept-1-yl)methanesulfonic acid salt. It is also approved as indicated as monotherapy for the treatment of patients with deleterious BRCA mutation (germline and/or somatic) associated advanced ovarian cancer who have been treated with two or more chemotherapies. The efficacy of rucaparib was investigated in 106 patients in two multicenter, single-arm, open-label clinical trials, Study 1 and Study 2, in patients with advanced BRCA-mutant ovarian cancer who had progressed after 2 or more prior chemotherapies. All 106 patients received rucaparib 600 mg orally twice daily as monotherapy until disease progression or unacceptable toxicity. Response assessment by independent radiology review was 42% (95% CI [32, 52]), with a median DOR of 6.7 months (95% CI [5.5, 11.1]). Investigator-assessed ORR was 66% (52/79; 95% CI [54, 76]) in platinum-sensitive patients, 25% (5/20; 95% CI [9, 49]) in platinum-resistant patients, and 0% (0/7; 95% CI [0, 41]) in platinum-refractory patients. ORR was similar for patients with a BRCA1 gene mutation or BRCA2 gene mutation. Thus, the rucaparib clinical data demonstrated that PARP inhibitors would not be beneficial to prolong progression free survival in the treatment of cancer characterized by the absence of mutations in BRCA1 or BRCA2.

Talazoparib

Similarly, talazoparib acts as an inhibitor of the enzyme poly ADP ribose polymerase (PARP), and is also termed a PARP inhibitor. It is currently being evaluated in clinical studies for the treatment of patients with gBRCA mutated breast cancer (i.e., advanced breast cancer in patients whose BRCA genes contain germline mutations). The primary objective of the study is to compare PFS of patients treated with talazoparib as a monotherapy relative to those treated with protocol-specified physicians' choice.

Veliparib

Similarly, veliparib acts as an inhibitor of the enzyme poly ADP ribose polymerase (PARP), and is also termed a PARP inhibitor. The chemical name of veliparib is 2-[(R)-2-methylpyrrolidin-2-yl]-1H-benzimidazole-4-carboxamide.

Cancers

The methods described herein can be useful for the treatment or prevention of cancer. Exemplary cancers are described herein.

The methods of the disclosure can be used to treat any type of cancer known in the art.

Non-limiting examples of cancers to be treated by the methods of the present disclosure can include melanoma (e.g., metastatic malignant melanoma), renal cancer (e.g. clear cell carcinoma), uterine cancers (e.g., uterine sarcoma or endometrial cancer), prostate cancer (e.g. hormone refractory prostate adenocarcinoma), gastrointestinal cancer, bladder cancer, pancreatic cancer, pancreatic adenocarcinoma, breast cancer, colon cancer, lung cancer (e.g. non-small cell lung cancer), esophageal cancer, squamous cell carcinoma, liver cancer, ovarian cancer, cervical cancer, thyroid cancer, head and neck cancer, glioblastoma, glioma, leukemia, lymphoma, mesothelioma, sarcoma and other neoplastic malignancies. Additionally, the invention includes refractory or recurrent malignancies whose growth may be inhibited using the methods of the invention. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, head and neck, tongue, larynx, and gullet), and adenocarcinoma (for example, prostate, small intestine, endometrium, cervical canal, large intestine, lung, pancreas, gullet, intestinum rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma.

In embodiments, a cancer is a cancer such as adenocarcinoma, adenocarcinoma of the lung, pancreatic adenocarcinoma, acute myeloid leukemia (“AML”), adrenocortical carcinoma, anal cancer, appendiceal cancer, B-cell derived leukemia, B-cell derived lymphoma, bladder cancer, brain cancer, breast cancer (e.g., triple negative breast cancer (TNBC)), cancer of the fallopian tube(s), cancer of the testes, cerebral cancer, cervical cancer, choriocarcinoma, chronic myelogenous leukemia, colon adenocarcinoma, colon cancer, colorectal cancer, diffuse large B cell lymphoma (“DLBCL”), endometrial cancer, epithelial cancer, esophageal cancer, Ewing's sarcoma, follicular lymphoma (“FL”), gall bladder cancer, gastric cancer, gastrointestinal cancer, glioma, head and neck cancer, a hematological cancer, hepatocellular cancer, Hodgkin's lymphoma/primary mediastinal B-cell lymphoma, kidney cancer, kidney clear cell cancer, laryngeal cancer, leukemia, liver cancer, lung cancer, lymphoma, melanoma, Merkel cell carcinoma, mesothelioma, monocytic leukemia, multiple myeloma, myeloma, a neuroblastic-derived CNS tumor, non-small cell lung cancer (NSCLC), oral cancer, ovarian cancer, ovarian carcinoma, pancreatic cancer, peritoneal cancer, primary peritoneal cancer, prostate cancer, relapsed or refractory classic Hodgkin's Lymphoma (cHL), renal cell carcinoma, rectal cancer, salivary gland cancer (e.g., a salivary gland tumor), sarcoma, skin cancer, small cell lung cancer, small intestine cancer, squamous cell carcinoma of the anogenital region, squamous cell carcinoma of the esophagus, squamous cell carcinoma of the head and neck (SCHNC), squamous cell carcinoma of the lung, stomach cancer, T-cell derived leukemia, T-cell derived lymphoma, thymic cancer, a thymoma, thyroid cancer, uveal melanoma, urothelial cell carcinoma, uterine cancer, uterine endometrial cancer, uterine sarcoma, vaginal cancer, or vulvar cancer.

In embodiments, a cancer is bladder cancer, breast cancer (e.g., triple negative breast cancer (TNBC)), cancer of the fallopian tube(s), cholagiocarcinoma, colon adenocarcinoma, endometrial cancer, esophageal cancer, Ewing's sarcoma, gastric cancer, kidney clear cell cancer, lung cancer (e.g., lung adenocarcinoma or lung squamous cell cancer), mesothelioma, ovarian cancer, pancreatic cancer, peritoneal cancer, prostate cancer, uterine endometrial cancer, or uveal melanoma. In embodiments, a cancer is ovarian cancer, cancer of the fallopian tube(s), or peritoneal cancer. In embodiments, a cancer is breast cancer (e.g., TNBC). In embodiments, a cancer is lung cancer (e.g., non-small cell lung cancer). In embodiments, a cancer is prostate cancer.

In embodiments, a cancer is a solid tumor such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non small cell lung cancer (NSCLC), small cell lung carcinoma, bladder carcinoma, lung cancer, epithelial carcinoma, skin cancer, melanoma, neuroblastoma, or retinoblastoma.

In embodiments, a cancer is a blood-borne cancer such as acute lymphoblastic leukemia (“ALL”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia and multiple myeloma; acute and chronic leukemias such as lymphoblastic, myelogenous, lymphocytic, and myelocytic leukemias.

In embodiments a cancer is a lymphoma such as Hodgkin's disease, non-Hodgkin's Lymphoma, multiple myeloma, Waldenstrom's macroglobulinemia, heavy chain disease, and polycythemia vera.

In embodiments, a cancer is a CNS or brain cancer such as glioma, pilocytic astrocytoma, astrocytoma, anaplastic astrocytoma, glioblastoma multiforme, medulloblastoma, craniopharyngioma, ependymoma, pinealoma, hemangioblastoma, acoustic neuroma, oligodendroglioma, meningioma, vestibular schwannoma, adenoma, metastatic brain tumor, meningioma, spinal tumor, or medulloblastoma.

In some embodiments, such cancers are selected from gynecologic cancers (i.e., cancers of the female reproductive system such as ovarian cancer, fallopian tube cancer, cervical cancer, vaginal cancer, vulvar cancer, uterine cancer, or primary peritoneal cancer). In some embodiments, cancers of the female reproductive system include, but are not limited to, ovarian cancer, cancer of the fallopian tube(s), peritoneal cancer and breast cancer. In some embodiments, an ovarian cancer is an epithelial carcinoma. Epithelial carcinomas make up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. In a normal female reproductive system, there are two fallopian tubes, one located on each side of the uterus. Cancer cells that begin in the fallopian tube may go to the surface of the ovary early on. The term “ovarian cancer” is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer develops in the egg-producing cells of the ovaries. In some embodiments, a cancer is or comprises a stromal tumor. Stromal tumors develop in the connective tissue cells that hold the ovaries together, which sometimes is the tissue that makes female hormones called estrogen. In some embodiments, a cancer is or comprises a granulosa cell tumor. Granulosa cell tumors may secrete estrogen resulting in unusual vaginal bleeding at the time of diagnosis. In some embodiments, a gynecologic cancer is associated with homologous recombination repair deficiency/homologous repair deficiency (“HRD”) and/or BRCA1/2 mutation(s). In some embodiments, a gynecologic cancer is platinum-sensitive. In some embodiments, a gynecologic cancer has responded to a platinum-based therapy. In some embodiments, a gynecologic cancer has developed resistance to a platinum-based therapy. In some embodiments, a gynecologic cancer has at one time shown a partial or complete response to platinum-based therapy (e.g., a partial or complete response to the last platinum-based therapy or to the penultimate platinum-based therapy). In some embodiments, a gynecologic cancer is now resistant to platinum-based therapy.

In embodiments, a cancer is metastatic. In some embodiments, a gynecological cancer (e.g., ovarian cancer) is metastatic. In some embodiments, a gynecological cancer (e.g., ovarian cancer) is an advanced gynecological cancer (e.g., ovarian cancer). In some embodiments, a cancer is a stage II, stage III or stage IV gynecological cancer (e.g., ovarian cancer).

In embodiments, a cancer is a recurrent cancer (e.g., a recurrent gynecological cancer such as recurrent epithelial ovarian cancer, recurrent fallopian tube cancer, or recurrent primary peritoneal cancer).

In embodiments, a cancer is an advanced cancer.

In embodiments, a cancer is characterized by a mutation in one or more genes. In some embodiments, the cancer is characterized by an ATM and/or BAP1 mutation.

In embodiments, a cancer is pancreatic cancer, melanoma, liver cancer, cervical cancer, gastric cancer, uterine cancer, or lung cancer. In some embodiments, a pancreatic cancer, melanoma, liver cancer, cervical cancer, gastric cancer, uterine cancer, or lung cancer is characterized by a bi-allelic mutation. In some embodiments, a pancreatic cancer, melanoma, liver cancer, cervical cancer, gastric cancer, uterine cancer, or lung cancer is characterized by a functional bi-allelic mutation.

In embodiments, a cancer is pancreatic cancer. In some embodiments, the pancreatic cancer is characterized by a BRCA2 mutation. In further embodiments, the BRCA2 mutation is bi-allelic.

In embodiments, a cancer is melanoma. In some embodiments, the melanoma is characterized by a BAP1 mutation. In further embodiments, the BAP1 mutation is bi-allelic.

In embodiments, a cancer is liver cancer. In some embodiments, the liver cancer is characterized by a BAP1 mutation. In further embodiments, the BAP1 mutation is bi-allelic.

In embodiments, a cancer is cervical cancer. In some embodiments, the cervical cancer is characterized by a BAP1 mutation. In further embodiments, the BAP1 mutation is bi-allelic.

In embodiments, a cancer is uterine cancer. In some embodiments, the uterine cancer is characterized by a BAP1 mutation. In further embodiments, the BAP1 mutation is bi-allelic. In some embodiments, the uterine cancer is characterized by a ATM mutation. In further embodiments, the ATM mutation is bi-allelic. In some embodiments, the uterine cancer is characterized by a BRCA1/2 mutation. In further embodiments, the BRCA1/2 mutation is bi-allelic.

In embodiments, a cancer is gastric cancer. In some embodiments, the gastric cancer is characterized by a BAP1 mutation. In further embodiments, the BAP1 mutation is bi-allelic.

Ovarian Cancer

Ovarian cancer begins when healthy cells in an ovary change and grow uncontrollably, forming a mass called a tumor. A tumor can be cancerous or benign. A cancerous tumor is malignant, meaning it can grow and spread to other parts of the body. A benign tumor means the tumor can grow but will not spread. Removing the ovary or the part of the ovary where the tumor is located can treat a noncancerous ovarian tumor. An ovarian cyst, which forms on the surface of the ovary, is different than a noncancerous tumor and usually goes away without treatment. A simple ovarian cyst is not cancerous. They often occur during the normal menstrual cycle. Types of ovarian cancer include: epithelial carcinoma, germ cell tumors, or stromal tumors.

Epithelial carcinoma makes up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. Every woman has two fallopian tubes, one located on each side of the uterus. Cancer cells that begin in the fallopian tube may go to the surface of the ovary early on. The term “ovarian cancer” is often used to describe epithelial cancers that begin in the ovary, in the fallopian tube, and from the lining of the abdominal cavity, called the peritoneum. A germ cell tumor is an uncommon type of ovarian cancer develops in the egg-producing cells of the ovaries. This type of tumor is more common in females ages 10 to 29. A stromal tumor is a rare form of ovarian cancer develops in the connective tissue cells that hold the ovaries together, which sometimes is the tissue that makes female hormones called estrogen. Over 90% of stromal tumors are adult or childhood granulosa cell tumors. Granulosa cell tumors may secrete estrogen resulting in unusual vaginal bleeding at the time of diagnosis.

The expected incidence of epithelial ovarian cancer in women in the United States in 2012 is approximately 22,280 (15,500 deaths) and in Europe in 2012 was estimated at 65,538 patient cases (42,704 deaths). At diagnosis, most women present with advanced disease, which accounts for the high mortality rate. Initial chemotherapy consists of either taxane or platinum chemotherapy or a combination of both. While approximately 75% of patients respond to front line therapy 70% of those eventually relapse within 1 to 3 years. There is a significant unmet need due to the high recurrence rate, despite an initially high response rate. Attempts to improve the standard two-drug chemotherapy (carboplatin and paclitaxel) by adding a third cytotoxic drug (topotecan, gemcitabine, or doxil) have failed (du Bois et al., “A phase I and pharmacokinetic study of novel taxane BMS-188797 and cisplatin in patients with advanced solid tumors”, Br. J. Cancer 94(1): 79-84 (2006); and Pfisterer et al., “Gemcitabine plus carboplatin compared with carboplatin in patients with platinum-sensitive recurrent ovarian cancer: an intergroup trial of the AGO-OVAR, the NCIC CTG, and the EORTC GCG”, J. Cin. Oncol. 24(29): 4699-707 (2006)). The great challenge for the near future will be the selection of patients with advanced ovarian cancer who will most benefit from specific targeted agents in the frontline maintenance setting. Maintenance therapy after the achievement of a response from initial chemotherapy may represent an approach to provide clinical benefit by delaying disease progression side effects, delaying the need for toxic chemotherapy and prolonging overall survival. However there is currently no widely accepted standard of care in the ovarian cancer maintenance setting.

The lack of successful treatment strategies led the Cancer Genome Atlas (TCGA) researchers to comprehensively measure genomic and epigenomic abnormalities on clinically annotated HGS-OvCa samples to identify molecular factors that influence pathophysiology affect outcome and constitute therapeutic targets (TCGA, 2011). Ovarian tumors are characterized by deficiencies in DNA repair such as BRCA mutations. BRCA 1 and 2 were initially identified as tumor suppressor genes that were associated with increased incidence of certain malignancies when defective, including ovarian cancer. BRCA deficiency was noted in 34% of ovarian cancers, owing to a combination of germline and sporadic mutations and promoter hypermethylation. BRCA plays a key role in DNA repair, including homologous recombination. This study estimated over half of high grade serous ovarian cancer suffered from defects in DNA repair. Tumor cells with BRCA deficiency/Homologous Recombination Deficiency (HRD) may provide an opportunity for therapeutic intervention with agents that inhibit DNA repair pathways and exploit synthetic lethality mechanisms of cancer treatment. Studies have suggested that HR deficiency in epithelial ovarian cancer (EOC) is not solely due to germline BRCA1 and BRCA2 mutations (Hennessy et al., “Somatic mutations in BRCA 1 and BRCA 2 could expand the number of patients that benefit from poly (ADP ribose) polymerase inhibitors in ovarian cancer”, J. Clin. Oncol. 28(22) 3570-76 (2010); TCGA, “Integrated genomic analyses of ovarian carcinoma”, Nature 474: 609-15 (2011); Byler Dann et al., “BRCA 1/2 mutations and expression: response to platinum chemotherapy in patients with advanced stage epithelial ovarian cancer”, Gynecol. Oncol. 125(3): 677-82 (2012)). The Cancer Genome Atlas Research Network (TCGA) reported a defect in at least one HR pathway gene in approximately half of the ˜500 EOC in the data set.

Patients having platinum-sensitive, recurrent ovarian cancer can benefit from methods of treatment described herein. Both the National Comprehensive Cancer Network (NCCN) and the European Society of Medical Oncology (ESMO) guidelines recommend re-treatment of patients with a platinum-based combination chemotherapy when relapse occurs >6 months after response to an initial platinum-based treatment. Paclitaxel plus carboplatin is the most frequently used regimen for platinum-sensitive patients who have recurred. Unfortunately, the utility of platinum-based chemotherapy diminishes over time; the PFS and platinum-free intervals generally become shorter after each subsequent treatment with tumors ultimately becoming platinum resistant or refractory. Furthermore, patients generally do not receive more than six (6) cycles of platinum-based chemotherapy per treatment course due to cumulative toxicities with platinum agents and taxanes. New agents and methods of treatment are needed to prolong the response to platinum-based chemotherapy, reduce the risk of recurrence or death, and increase the platinum-free interval.

In embodiments, an ovarian cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2, and optionally an identified deficiency in BRCA1 and/or BRCA2) has recurrent ovarian cancer (including fallopian and peritoneal cancers). Alternatively, or in addition to, the ovarian cancer patient has a deficiency one or more of the genes TP3 and/or RB1. In embodiments, a non-BRCA1/2 HRR deficiency is in one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L, and optionally a deficiency in BRCA1 and/or BRCA2. Alternatively, or in addition to, the ovarian cancer patient has a deficiency one or more of the genes TP3 and/or RB1. In embodiments, a non-BRCA1/2 HRR deficiency is in one or more, two or more, three or more, four or more, five or more, seven or more, eight or more, nine or more, ten or more, eleven or more genes, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2, and optionally a deficiency in BRCA1 and/or BRCA2. Alternatively, or in addition to, the ovarian cancer patient has a deficiency one or more of the genes TP3 and/or RB1.

In embodiments, a PARP inhibitor (e.g., niraparib) is administered as a maintenance therapy. In embodiments, said administration of a PARP inhibitor (e.g., niraparib) results in prolongation of progression free survival.

In embodiments, a PARP inhibitor (e.g., niraparib) is administered as a monotherapy for the maintenance treatment for a cancer patient who is in response to platinum-based chemotherapy (e.g., a partial response or a complete response). In one embodiment, a PARP inhibitor (e.g., niraparib) is administered as a monotherapy for the maintenance treatment of a patient further having deleterious or suspected deleterious germline or somatic BRCA mutation(s). In another embodiment, a patient with recurrent ovarian cancer is further characterized by the absence of a germline BRCA mutation that is deleterious or suspected to be deleterious.

In embodiments, a PARP inhibitor (e.g., niraparib) is administered as a maintenance therapy in patients with recurrent ovarian cancer (including fallopian and peritoneal cancers) who have a complete response or partial response following at least one platinum-based chemotherapy treatment. In embodiments, a PARP inhibitor (e.g., niraparib) is administered as a maintenance therapy in patients with recurrent ovarian cancer (including fallopian and peritoneal cancers) who have a complete response or partial response following multiple platinum-based chemotherapy treatment (e.g., at least two, or least three, at least four, at least five, or at least six platinum-based chemotherapy treatments). In embodiments, a patient has a complete or partial response to the most recent platinum-based chemotherapy treatment. In embodiments, a patient has a complete or partial response to the penultimate platinum-based chemotherapy treatment. In embodiments, said administration of a PARP inhibitor (e.g., niraparib) results in prolongation of progression free survival. Such a prolongation of progression free survival may result in a reduced hazard ratio for disease progression or death. In embodiments, maintenance therapy is administered during the interval between cessation of chemotherapy with the goal of delaying disease progression and the subsequent intensive therapies that may present tolerability issues for patients. In another embodiment, a patient with recurrent ovarian cancer is further characterized as having a BRCA deficiency. In another embodiment, a patient with recurrent ovarian cancer is further characterized by the absence of a germline BRCA mutation that is deleterious or suspected to be deleterious.

In another embodiment, a second approach to address the high recurrence rate of ovarian cancers is to select patients with advanced ovarian cancer who will most benefit from specific targeted agents in the frontline therapy or maintenance setting. In embodiments, an ovarian cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2) has advanced ovarian cancer. Alternatively, or in addition to, the ovarian cancer patient has a deficiency one or more of the genes TP3 and/or RB1.

Accordingly, a PARP inhibitor (e.g., niraparib) is administered as a therapy in patients with advanced ovarian cancer, wherein said administration results in an increase in overall survival and wherein administration is either as a treatment (in the case of continued disease following 1-4 prior lines of therapy) or a maintenance treatment (in the case of a patient with a PR or CR to a prior therapy). In another embodiment, the patients with advanced ovarian cancer are further characterized as having a further deficiency that is a BRCA deficiency. In another embodiment, the patients with advanced ovarian cancer are further characterized by the absence of a germline BRCA mutation that is deleterious or suspected to be deleterious.

In embodiments, an ovarian cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2) has recurrent or platinum sensitive ovarian cancer, fallopian tube cancer, or primary peritoneal cancer. Alternatively, or in addition to, the ovarian cancer patient has a deficiency one or more of the genes TP3 and/or RB1. In embodiments, a non-BRCA1/2 HRR deficiency is in one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L, and optionally a deficiency in BRCA1 and/or BRCA2. Alternatively, or in addition to, the ovarian cancer patient has a deficiency one or more of the genes TP3 and/or RB1. In embodiments, a non-BRCA1/2 HRR deficiency is in one or more, two or more, three or more, four or more, five or more, seven or more, eight or more, nine or more, ten or more, eleven or more genes, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2, and optionally a deficiency in BRCA1 and/or BRCA2. Alternatively, or in addition to, the ovarian cancer patient has a deficiency one or more of the genes TP3 and/or RB1.

In some embodiments, the present invention provides a method of administering a PARP inhibitor (e.g., niraparib) to a patient having recurrent or platinum sensitive ovarian cancer, fallopian tube cancer, or primary peritoneal cancer comprising administering niraparib according to a regimen determined to achieve prolonged progression free survival (e.g., a regimen as described herein). In some embodiments, the progression free survival is greater in patients receiving a PARP inhibitor (e.g., niraparib), for example as compared with patients not receiving a PARP inhibitor (e.g., niraparib). In some embodiments, progression free survival is greater in patients receiving a PARP inhibitor (e.g., niraparib) than in patients receiving alternative cancer therapy, for example such as therapy with niraparib as compared with a different PARP inhibitor.

Breast Cancer

In embodiments, a breast cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2) has breast cancer. Alternatively, or in addition to, the breast cancer patient has a deficiency one or more of the genes TP3 and/or RB1. In embodiments, a non-BRCA1/2 HRR deficiency is in one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L, and optionally a deficiency in BRCA1 and/or BRCA2. Alternatively, or in addition to, the breast cancer patient has a deficiency one or more of the genes TP3 and/or RB1. In embodiments, a non-BRCA1/2 HRR deficiency is in one or more, two or more, three or more, four or more, five or more, seven or more, eight or more, nine or more, ten or more, eleven or more genes, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2, and optionally a deficiency in BRCA1 and/or BRCA2. Alternatively, or in addition to, the breast cancer patient has a deficiency one or more of the genes TP3 and/or RB1.

Usually breast cancer either begins in the cells of the milk producing glands, known as the lobules, or in the ducts. Less commonly breast cancer can begin in the stromal tissues. These include the fatty and fibrous connective tissues of the breast. Over time the breast cancer cells can invade nearby tissues such the underarm lymph nodes or the lungs in a process known as metastasis. The stage of a breast cancer, the size of the tumor, and its rate of growth are all factors which determine the type of treatment that is offered. Treatment options include surgery to remove the tumor, drug treatment, which includes chemotherapy and hormonal therapy, radiation therapy, and immunotherapy. The prognosis and survival rate varies widely; the five year relative survival rates vary from 98% to 23% depending on the type of breast cancer that occurs. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative.

In some embodiments, a breast cancer is a metastatic breast cancer. In some embodiments, a breast cancer is an advanced breast cancer. In some embodiments, a cancer is a stage II, stage III or stage IV breast cancer. In some embodiments, a cancer is a stage IV breast cancer. In some embodiments, a breast cancer is a triple negative breast cancer.

Lung Cancer

In embodiments, a cancer is a lung cancer.

Lung cancer is the most common cause of cancer mortality globally and the second most common cancer in both men and women. About 14% of all new cancers are lung cancers. In the United States (US), there are projected to be 222,500 new cases of lung cancer (116,990 in men and 105,510 in women) and 155,870 deaths from lung cancer (84,590 in men and 71,280 in women) in 2017.

The two major forms of lung cancer are non-small cell lung cancer (NSCLC) and small cell lung cancer. NSCLC is a heterogeneous disease that consists of adenocarcinoma, large-cell carcinoma, and squamous cell carcinoma (sqNSCLC), and comprises approximately 80% to 85% of all lung cancers. Squamous cell carcinoma of the lung accounts for 20% to 30% of NSCLC. Despite advances in early detection and standard treatment, NSCLC is often diagnosed at an advanced stage, has poor prognosis, and is the leading cause of cancer deaths worldwide.

Platinum-based doublet therapy, maintenance chemotherapy, and anti-angiogenic agents in combination with chemotherapy have contributed to improved patient outcomes in advanced NSCLC. The identification of certain point mutations (e.g., epidermal growth factor receptor [EGFR], BRAF), gene fusions due to chromosomal translocations (e.g., anaplastic lymphoma kinase [ALK], ROS-1), and gene amplifications (e.g., mesenchymal epithelial transition factor [MET]) have been shown to serve as oncogenic drivers in providing treatment to the cancer patient. See, e.g., U.S. Provisional Application No. 62/726,826. For most NSCLC patients without targetable oncogene drivers, first-line platinum-based chemotherapy was until recently the only standard treatment approach.

In embodiments, a lung cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2) has lung cancer. Alternatively, or in addition to, the deficiency is in one or more of the genes TP3 and/or RB1. In embodiments, a non-BRCA1/2 HRR deficiency is in one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L, and optionally a deficiency in BRCA1 and/or BRCA2. Alternatively, or in addition to, the deficiency is in one or more of the genes TP3 and/or RB1. In embodiments, a non-BRCA1/2 HRR deficiency is in one or more, two or more, three or more, four or more, five or more, seven or more, eight or more, nine or more, ten or more, eleven or more genes, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2, and optionally a deficiency in BRCA1 and/or BRCA2. Alternatively, or in addition to, the HRR deficiency is in one or more of the genes TP3 and/or RB1.

In embodiments, the lung cancer is non-small cell lung cancer (NSCLC) (e.g., NSCLC that is high PD-L1 expressing or low PD-L1 expressing). In embodiments, a lung cancer is squamous NSCLC.

In embodiments, a lung cancer is recurrent as described herein (e.g., a recurrent non-small cell lung cancer (NSCLC)).

In embodiments, a lung cancer is an advanced lung cancer. In embodiments, a lung cancer is a metastatic lung cancer. In embodiments, a lung cancer is squamous cell carcinoma of the lung. In embodiments, a lung cancer is small cell lung cancer (SCLC). In embodiments, a lung cancer is non-small cell lung cancer (NSCLC). In embodiments, a lung cancer is an ALK-translocated lung cancer (e.g., a lung cancer with a known ALK-translocation). In embodiments, a lung cancer is an EGFR-mutant lung cancer (e.g., a lung cancer with a known EGFR mutation). In embodiments, a lung cancer is a MSI-H lung cancer. In embodiments, a lung cancer is a MSS lung cancer. In embodiments, a lung cancer is a POLE-mutant lung cancer. In embodiments, a lung cancer is a POLD-mutant lung cancer. In embodiments, a lung cancer is a high TMB lung cancer. In embodiments, a lung cancer is associated with homologous recombination repair deficiency/homologous repair deficiency (“HRD”) or is characterized by a homologous recombination repair (HRR) gene mutation or deletion.

In embodiments, an advanced lung cancer (e.g., advanced NSCLC) is stage III cancer or stage IV cancer. In embodiments, an advanced lung cancer (e.g., advanced NSCLC) is stage III cancer. In embodiments, an advanced lung cancer (e.g., advanced NSCLC) is stage IV cancer. In embodiments, an advanced lung cancer (e.g., advanced NSCLC) is locally advanced. In embodiments, an advanced lung cancer (e.g., advanced NSCLC) is metastatic.

In embodiments, a subject having lung cancer (e.g., NSCLC such as advanced NSCLC) is treatment-naïve for the lung cancer. In embodiments, a subject having lung cancer (e.g., NSCLC such as advanced NSCLC) is treatment-naïve for the lung cancer and has not previously received immunotherapy (e.g., anti-PD-1 therapy) nor chemotherapy. In embodiments, a subject having lung cancer (e.g., NSCLC such as advanced NSCLC) is treatment-naïve for the lung cancer and has not previously received immunotherapy. In embodiments, a subject having lung cancer (e.g., NSCLC such as advanced NSCLC) is treatment-naïve for the lung cancer and has not previously received an anti-PD-1 therapy (“PD-1-naïve”). In embodiments, a subject having lung cancer (e.g., NSCLC such as advanced NSCLC) is treatment-naïve for the lung cancer and has not previously received chemotherapy (“chemotherapy-naïve”). In embodiments, a subject having lung cancer (e.g., NSCLC such as advanced NSCLC) is treatment-naïve for the lung cancer and has not previously received chemotherapy such as platinum-based chemotherapy or chemotherapy comprising an inhibitor of EGFR, ALK, ROS-1, and/or MET.

In embodiments, a lung cancer (e.g., NSCLC such as advanced NSCLC) does not express PD-L1.

In embodiments, a lung cancer (e.g., NSCLC such as advanced NSCLC) expresses PD-L1 (e.g., as determined by an assay such as an immunohistochemical (IHC) assay). In embodiments, a lung cancer (e.g., NSCLC such as advanced NSCLC) expresses ≥1% PD-L1 (e.g., as determined by an assay such as an immunohistochemical (IHC) assay). In embodiments, a lung cancer (e.g., NSCLC such as advanced NSCLC) expresses ≥50% PD-L1 (e.g., as determined by an assay such as an immunohistochemical (IHC) assay). In embodiments, a lung cancer (e.g., NSCLC such as advanced NSCLC) is a high PD-L1 cancer (e.g., a cancer that expresses ≥50% PD-L1 (e.g., as determined by an assay such as an immunohistochemical (IHC) assay)).

In embodiments, a lung cancer is small cell lung cancer (SCLC).

In embodiments, a lung cancer is non-small cell lung cancer (NSCLC) such as adenocarcinoma, large-cell carcinoma, or squamous cell carcinoma (sqNSCLC). In embodiments, a NSCLC is lung adenocarcinoma. In embodiments, a NSCLC is large cell carcinoma of the lung. In embodiments, a NSCLC is squamous cell carcinoma of the lung (sqNSCLC).

In embodiments, a lung cancer is an ALK-translocated lung cancer (e.g., ALK-translocated NSCLC). In embodiments, a cancer is NSCLC (e.g., advanced NSCLC) with an identified ALK translocation.

In embodiments, a lung cancer (e.g., NSCLC such as advanced NSCLC) does not have an ALK-translocation. In embodiments, a cancer is NSCLC (e.g., advanced NSCLC) without ALK translocation.

In embodiments, a lung cancer (e.g., NSCLC such as advanced NSCLC) is an EGFR-mutant lung cancer (e.g., EGFR-mutant NSCLC). In embodiments, a cancer is NSCLC (e.g., advanced NSCLC) with an identified EGFR mutation.

In embodiments, a lung cancer (e.g., NSCLC such as advanced NSCLC) does not have an EGFR mutation. In embodiments, a cancer is NSCLC (e.g., advanced NSCLC) without an EGFR mutation.

In embodiments, a lung cancer (e.g., NSCLC such as advanced NSCLC) is an ROS-1-translocated lung cancer (e.g., ROS-1-translocated NSCLC). In embodiments, a cancer is NSCLC (e.g., advanced NSCLC) with an identified ROS-1 translocation.

In embodiments, a lung cancer (e.g., NSCLC such as advanced NSCLC) does not have an ROS-1-translocation. In embodiments, a cancer is NSCLC (e.g., advanced NSCLC) without ROS-1 translocation.

In embodiments, a lung cancer (e.g., NSCLC such as advanced NSCLC) is characterized by a gene amplification (e.g., in mesenchymal epithelial transition factor (MET)). In embodiments, a cancer is NSCLC (e.g., advanced NSCLC) characterized by a MET amplification.

In embodiments, a lung cancer (e.g., NSCLC such as advanced NSCLC) is characterized by an EGFR mutation, an ALK translocation, a ROS-1 translocation, and/or a gene amplification in mesenchymal epithelial transition factor (MET).

In embodiments, a lung cancer (e.g., NSCLC such as advanced NSCLC) does not have an EGFR mutation, an ALK translocation, a ROS-1 translocation, nor a gene amplification in mesenchymal epithelial transition factor (MET).

In embodiments, a lung cancer (e.g., NSCLC such as advanced NSCLC) is not characterized by a gene amplification. In embodiments, a cancer is NSCLC (e.g., advanced NSCLC) that is not characterized by a gene amplification. In embodiments, a cancer is NSCLC (e.g., advanced NSCLC) that is not characterized by a gene amplification in mesenchymal epithelial transition factor (MET).

In embodiments, a subject is treatment-naïve (e.g., chemotherapy-naïve and/or PD-1-naïve). In embodiments, a treatment-naïve subject has not previously received chemotherapy (e.g., chemotherapy that is platinum-based chemotherapy and/or an inhibitor of any of EGFR, ALK, ROS-1, and MET) nor a previous anti-PD-1 therapy (e.g., anti-PD-1 therapy that is an inhibitor of PD-1 and/or PD-L1/L2). In embodiments, a lung cancer (e.g., NSCLC such as advanced NSCLC) is advanced. In embodiments, an advanced lung cancer (e.g., advanced NSCLC) is locally advanced. In embodiments, an advanced lung cancer (e.g., advanced NSCLC) is metastatic. In embodiments, a lung cancer (e.g., NSCLC such as advanced NSCLC) expresses PD-L1. In embodiments, a lung cancer (e.g., NSCLC such as advanced NSCLC) is high PD-L1 (e.g., TPS≥50%). In embodiments, PD-L1 expression is determined using an immunohistochemical (IHC) assay.

In embodiments, a lung cancer is characterized by a HRR deficiency as described herein (e.g., a deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2. Alternatively, or in addition to, the lung cancer is characterized by a deficiency one or more of the genes TP3 and/or RB1.

In embodiments, a lung cancer is characterized by a ATM deficiency. In embodiments, a ATM deficiency results from a bi-allelic mutation.

Pancreatic Cancer

In embodiments, a cancer is pancreatic cancer.

Pancreatic cancer continues to have one of the highest mortality rates of any malignancy. Each year, 28,000 patients are diagnosed with pancreatic cancer, and most will die of the disease. The vast majority of patients are diagnosed at an advanced stage of disease because currently no tumor markers are known that allow reliable screening for pancreas cancer at an earlier, potentially curative stage. This is a particular problem for those patients with a strong familial history of pancreatic cancer, who may have up to a 5-7 fold greater risk of developing pancreatic cancer in their lifetime. Despite several advances in our basic understanding and clinical management of pancreatic cancer, virtually all patients who will be diagnosed with pancreatic cancer will die from this disease. The high mortality of pancreatic cancer is predominantly due to consistent diagnosis at an advanced stage of disease, and a lack of effective screening methods.

Pancreatic cancer encompasses benign or malignant forms of pancreatic cancer, as well as any particular type of cancer arising from cells of the pancreas. In embodiments, a pancreatic cancer is duct cell carcinoma, acinar cell carcinoma, papillary carcinoma, adenosquamous carcinoma, undifferentiated carcinoma, mucinous carcinoma, giant cell carcinoma, mixed type pancreatic cancer, small cell carcinoma, cystadenocarcinoma, an unclassified pancreatic cancer, pancreatoblastoma, or papillary-cystic neoplasm.

The many types of pancreatic cancer can be divided into two general groups. The vast majority of cases (about 95%) occur in the part of the pancreas which produces digestive enzymes, known as the exocrine component. Cancers that arise in the hormone-producing (endocrine) tissue of the pancreas can have different clinical characteristics and are called pancreatic neuroendocrine tumors, sometimes abbreviated as “PanNETs”. Both groups occur mainly (but not exclusively) in people over 40, and are slightly more common in men, but some rare sub-types mainly occur in women or children.

In embodiments, a pancreatic cancer is an exocrine-type pancreatic cancer. Exemplary exocrine-type pancreatic cancers include pancreatic adenocarcinoma, acinar cell carcinoma of the pancreas, cystadenocarcinomas, pancreatoblastoma, adenosquamous carcinomas, signet ring cell carcinomas, hepatoid carcinomas, colloid carcinomas, undifferentiated carcinomas, undifferentiated carcinomas with osteoclast-like giant cells. solid pseudopapillary tumor, and pancreatic mucinous cystic neoplasms. In embodiments, an exocrine cancer is selected from adenosquamous carcinomas, signet ring cell carcinomas, hepatoid carcinomas, colloid carcinomas, undifferentiated carcinomas, and undifferentiated carcinomas with osteoclast-like giant cells.

In embodiments, a pancreatic cancer is duct cell carcinoma, acinar cell carcinoma, papillary carcinoma, adenosquamous carcinoma, undifferentiated carcinoma, mucinous carcinoma, giant cell carcinoma, mixed type pancreatic cancer, small cell carcinoma, cystadenocarcinoma, unclassified pancreatic cancers, pancreatoblastoma, papillary-cystic neoplasm, or the like, or a combination thereof.

In embodiments, a pancreatic cancer is pancreatic adenocarcinoma (variations of this name may add “invasive” and “ductal”), which represents about 85% of exocrine pancreatic cancers. Nearly all these start in the ducts of the pancreas, as pancreatic ductal adenocarcinoma (PDAC). About 60-70% of adenocarcinomas occur in the head of the pancreas.

In embodiments, a pancreatic cancer is acinar cell carcinoma of the pancreas, which arises in the clusters of cells that produce these enzymes, and represents 5% of exocrine pancreas cancers.

In embodiments, a pancreatic cancer is a cystadenocarcinoma, which accounts for 1% of pancreatic cancers.

In embodiments, a pancreatic cancer is pancreatoblastoma.

In embodiments, a pancreatic cancer is a solid pseudopapillary tumor.

In embodiments, a pancreatic cancer is a pancreatic mucinous cystic neoplasm.

In embodiments, the pancreatic cancer is a neuroendocrine-type pancreatic cancer. Exemplary neuroendocrine-type pancreatic cancers include islet cell carcinomas (e.g., insulinoma, gastrinoma, VIPoma, glucagonoma, somatostatinoma, PPoma, ACTHoma, CRHoma, calcitoninoma, GHRHoma, GRFoma, parathyroid hormone-related peptide tumor).

In embodiments, the pancreatic cancer patient is human. In embodiments, the pancreatic cancer patient is male. In embodiments, the pancreatic cancer patient is a female (e.g., a young female). In embodiments, the pancreatic cancer patient is a child.

In some embodiments, a pancreatic cancer is a metastatic pancreatic cancer. In some embodiments, a pancreatic cancer is an advanced pancreatic cancer. In some embodiments, a cancer is a stage II, stage III, or stage IV pancreatic cancer.

In embodiments, a pancreatic cancer is characterized by a HRR deficiency as described herein (e.g., a deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2. Alternatively, or in addition to, the pancreatic cancer is characterized by a deficiency one or more of the genes TP3 and/or RB1.

In embodiments, a pancreatic cancer is characterized by a BRCA1/2 deficiency. In embodiments, a pancreatic cancer characterized by a BRCA1 deficiency. In embodiments, a BRCA1 deficiency results from a monoallelic mutation. In embodiments, a BRCA1 deficiency results from a bi-allelic mutation or a functional bi-allelic mutation. In embodiments, a pancreatic cancer characterized by a BRCA2 deficiency. In embodiments, a BRCA2 deficiency results from a monoallelic mutation. In embodiments, a BRCA2 deficiency results from a bi-allelic mutation or a functional bi-allelic mutation.

Recurrent Cancers

In embodiments, a cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2) has a recurrent cancer. Alternatively, or in addition to, the cancer patient has a deficiency one or more of the genes TP3 and/or RB1. In embodiments, a non-BRCA1/2 HRR deficiency is in one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L, and optionally a deficiency in BRCA1 and/or BRCA2. Alternatively, or in addition to, the deficiency is in one or more of the genes TP3 and/or RB1. In embodiments, a non-BRCA1/2 HRR deficiency is in one or more, two or more, three or more, four or more, five or more, seven or more, eight or more, nine or more, ten or more, eleven or more genes, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2, and optionally a deficiency in BRCA1 and/or BRCA2. Alternatively, or in addition to, the deficiency is in one or more of the genes TP3 and/or RB1.

In embodiments, a PARP inhibitor (e.g., niraparib) is administered as a maintenance therapy.

In one embodiment, a PARP inhibitor (e.g., niraparib) is administered as a maintenance therapy to a patient with a recurrent cancer. In embodiments, administration of a PARP inhibitor (e.g., niraparib) results in prolongation of progression free survival. In one embodiment, a PARP inhibitor (e.g., niraparib) is administered as a monotherapy for the maintenance treatment of patients with a recurrent cancer. In one embodiment, a PARP inhibitor (e.g., niraparib) is administered as a monotherapy for the maintenance treatment of patients characterized by a further deficiency that is deleterious or suspected deleterious germline or somatic BRCA mutation(s).

In embodiments, a patient with a recurring cancer has undergone at least one cycle of a platinum-based chemotherapy. In embodiments, a cancer patient is in response (e.g., partial or complete response) to platinum-based chemotherapy. In embodiments, a patient with a recurring cancer has undergone at least two cycles of a platinum-based chemotherapy. In embodiments, a cancer is platinum-sensitive. In embodiments, a cancer patient has a complete response to the most recent platinum-based chemotherapy. In embodiments, a cancer patient has a partial response to the most recent platinum-based chemotherapy. In embodiments, a cancer patient has a complete response to the penultimate platinum-based chemotherapy. In embodiments, a cancer patient has a partial response to the penultimate platinum-based chemotherapy.

In one embodiment, a PARP inhibitor (e.g., niraparib) is administered as a maintenance therapy in patients with recurrent ovarian cancer (including fallopian and peritoneal cancers). In embodiments, administration of a PARP inhibitor (e.g., niraparib) results in prolongation of progression free survival. In one embodiment, a PARP inhibitor (e.g., niraparib) is administered as a monotherapy for the maintenance treatment of patients with recurrent ovarian, fallopian tube, or primary peritoneal cancer, wherein the patient is in response to platinum-based chemotherapy. In one embodiment, a PARP inhibitor (e.g., niraparib) is administered as a monotherapy for the maintenance treatment of patients characterized by a further deficiency that is deleterious or suspected deleterious germline or somatic BRCA mutation(s). In embodiments, a cancer patient is in response to platinum-based chemotherapy.

Such a prolongation of progression free survival may result in a reduced hazard ratio for disease progression or death. Maintenance therapy is administered during the interval between cessation of initial therapy with the goal of delaying disease progression and the subsequent intensive therapies that may present tolerability issues for patients. In another embodiment, the patients with recurrent ovarian cancer are further characterized as having a BRCA deficiency. In another embodiment, the patients with recurrent ovarian cancer are further characterized by the absence of a germline BRCA mutation that is deleterious or suspected to be deleterious.

In one embodiment, a PARP inhibitor (e.g., niraparib) is administered as a maintenance therapy in patients with recurrent ovarian cancer (including fallopian and peritoneal cancers) who have a complete response or partial response following at least one platinum-based chemotherapy treatment. In one embodiment, a PARP inhibitor (e.g., niraparib) is administered as a maintenance therapy in patients with recurrent ovarian cancer (including fallopian and peritoneal cancers) who have a complete response or partial response following multiple platinum-based chemotherapy treatment (e.g., at least two, or least three, at least four, at least five, or at least six platinum-based chemotherapy treatments). In embodiments, a patient has a complete or partial response to the most recent platinum-based chemotherapy treatment. In embodiments, a patient has a complete or partial response to the penultimate platinum-based chemotherapy treatment. In embodiments, administration of a PARP inhibitor (e.g., niraparib) results in prolongation of progression free survival. Such a prolongation of progression free survival may result in a reduced hazard ratio for disease progression or death. Maintenance therapy is administered during the interval between cessation of chemotherapy with the goal of delaying disease progression and the subsequent intensive therapies that may present tolerability issues for patients. In another embodiment, the patients with recurrent ovarian cancer are further characterized as having a further deficiency that is a BRCA deficiency. In another embodiment, the patients with recurrent ovarian cancer are further characterized by the absence of a germline BRCA mutation that is deleterious or suspected to be deleterious.

In embodiments, a cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2) has recurrent or platinum sensitive ovarian cancer, fallopian tube cancer, or primary peritoneal cancer. Alternatively, or in addition to, the cancer patient has a deficiency is in one or more of the genes TP3 and/or RB1.

In some embodiments, the present invention provides a method of administering niraparib to a patient having recurrent or platinum sensitive ovarian cancer, fallopian tube cancer, or primary peritoneal cancer comprising administering a PARP inhibitor (e.g., niraparib). In embodiments, a PARP inhibitor (e.g., niraparib) is administered according to a regimen determined to achieve prolonged progression free survival. In some embodiments, the progression free survival is greater in patients receiving niraparib, for example as compared with patients not receiving niraparib. In some embodiments, progression free survival is greater in patients receiving niraparib than in patients receiving alternative cancer therapy, for example such as therapy with a different PARP inhibitor.

PD-L1 Negative Cancer

In some aspects and in some embodiments of the disclosure, the cancer is PD-L1 negative. As will be understood by one of skill in the art, a subject having a cancer that is PD-L1 negative means that the expression of PD-L1 is reduced or absent in a cancer cell in the subject. PD-L1 expression may be measured by any method known to one of skill in the art. For example, PD-L1 expression may be measured by immunohistochemistry (HC) using the PD-L1 IC 22C3 pharmDx (Agilent, Carpinteria, Calif., USA). In some embodiments, a cancer is PD-L1 negative if expression in cancer cells compared to immune cells by IHC is 1% or less.

Prolonged Progression Free Survival

In embodiments, methods described herein comprise administering a PARP inhibitor (e.g., niraparib) according to a regimen determined to achieve prolonged progression free survival in a cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2). Alternatively, or in addition to, the cancer patient has a deficiency is in one or more of the genes TP3 and/or RB1. In embodiments, a non-BRCA1/2 HRR deficiency is in one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L, and optionally a deficiency in BRCA1 and/or BRCA2. Alternatively, or in addition to, the deficiency is in one or more of the genes TP3 and/or RB1. In embodiments, a non-BRCA1/2 HRR deficiency is in one or more, two or more, three or more, four or more, five or more, seven or more, eight or more, nine or more, ten or more, eleven or more genes, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2, and optionally a deficiency in BRCA1 and/or BRCA2. Alternatively, or in addition to, the deficiency is in one or more of the genes TP3 and/or RB1.

In some embodiments, the progression free survival is greater in patients receiving a PARP inhibitor (e.g., niraparib), for example as compared with patients not receiving a PARP inhibitor (e.g., niraparib). In some embodiments, progression free survival is greater in patients receiving a PARP inhibitor (e.g., niraparib) than in patients receiving alternative cancer therapy (e.g., patients receiving niraparib have a greater progression free survival than patients receiving therapy with a different PARP inhibitor). In embodiments, a patient has recurrent or platinum sensitive ovarian cancer, fallopian tube cancer, or primary peritoneal cancer. In embodiments, the patient has high grade serous ovarian cancer or high grade predominantly serous histology ovarian cancer. In embodiments, a patient has non-small cell lung cancer (NSCLC).

In some embodiments, the prolonged progression free survival is at least 6 months. In some embodiments, the prolonged progression free survival is at least 9 months. In some embodiments, the prolonged progression free survival is at least 10 months. In some embodiments, the prolonged progression free survival is at least 11 months. In some embodiments, the progression free survival is at least 12 months. In some embodiments, the progression free survival is at least 15 months. In some embodiments, the progression free survival is at least 18 months. In some embodiments, the progression free survival is at least 21 months. In some embodiments, the progression free survival is at least 24 months. In some embodiments, the progression free survival is at least 27 months. In some embodiments, the progression free survival is at least 30 months. In some embodiments, the progression free survival is at least 33 months. In some embodiments, the progression free survival is at least 36 months.

In some embodiments, the methods prolong progression free survival as compared to control.

In embodiments, the patient is further characterized by an absence of a germline mutation in BRCA1 or BRCA2. In embodiments, the patient is further characterized by an absence of a sporadic mutation in BRCA1 or BRCA2. In embodiments, the patient is further characterized by a negative BRCA1/2 status. In embodiments, a germline mutation in BRCA1 or BRCA2 is not detected in a sample from a patient. In embodiments, the population of subjects exhibits non-mutated BRCA1/2 “BRCAwt” or “BRCAwt”.

In embodiments, the population of subjects has a BRCA mutation. In some embodiments, the patient also has at least (i) a germline mutation in BRCA1 or BRCA2 or (ii) a sporadic mutation in BRCA1 or BRCA2. In embodiments, the BRCA mutation is a germline BRCA mutation (gBRCAmut). In embodiments, the BRCA mutation is a somatic (or sporadic) BRCA mutation (sBRCAmut).

In some embodiments, the patient also has a germline mutation in BRCA1 and/or BRCA2 (gBRCAmut). In some embodiments, the prolonged progression free survival is at least 9-months. In some embodiments, the prolonged progression free survival is at least 10-months. In some embodiments, the prolonged progression free survival is at least 11-months. In some embodiments, the prolonged progression free survival is at least 12-months. In some embodiments, the prolonged progression free survival is at least 15-months. In some embodiments, the prolonged progression free survival is at least 18-months. In some embodiments, the prolonged progression free survival is at least 21-months. In some embodiments, the prolonged progression free survival is at least 24-months. In some embodiments, the prolonged progression free survival is at least 27-months. In some embodiments, the prolonged progression free survival is at least 30-months. In some embodiments, the prolonged progression free survival is at least 33-months. In some embodiments, the prolonged progression free survival is at least 36-months.

In some embodiments, the patient is characterized by an absence of a mutation in BRCA1 and/or BRCA2 (BRCAwt). In some embodiments, the prolonged progression free survival is at least 3-months. In some embodiments, the prolonged progression free survival is at least 6-months. In some embodiments, the prolonged progression free survival is at least 9-months. In some embodiments, the prolonged progression free survival is at least 10-months. In some embodiments, the prolonged progression free survival is at least 11-months. In some embodiments, the prolonged progression free survival is at least 12-months. In some embodiments, the prolonged progression free survival is at least 15-months. In some embodiments, the prolonged progression free survival is at least 18-months. In some embodiments, the prolonged progression free survival is at least 21-months. In some embodiments, the prolonged progression free survival is at least 24-months. In some embodiments, the prolonged progression free survival is at least 27-months. In some embodiments, the prolonged progression free survival is at least 30-months. In some embodiments, the prolonged progression free survival is at least 33-months. In some embodiments, the prolonged progression free survival is at least 36-months.

Hazard Ratios

In embodiments, methods described herein comprise administering a PARP inhibitor (e.g., niraparib) according to a regimen determined to achieve a hazard ratio for disease progression or death in a cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2). Alternatively, or in addition to, the cancer patient has a deficiency is in one or more of the genes TP3 and/or RB1.

In some embodiments, the hazard ratio is improved in patients receiving a PARP inhibitor (e.g., niraparib), for example as compared with patients not receiving the PARP inhibitor (e.g., niraparib). In some embodiments, the hazard ratio is improved in patients receiving niraparib than in patients receiving alternative cancer therapy (e.g., patients receiving niraparib have a greater progression free survival than patients receiving therapy with a different PARP inhibitor). In embodiments, a patient has recurrent or platinum sensitive ovarian cancer, fallopian tube cancer, or primary peritoneal cancer. In embodiments, the patient has high grade serous ovarian cancer or high grade predominantly serous histology ovarian cancer. In embodiments, a patient has non-small cell lung cancer (NSCLC).

In some embodiments, the hazard ratio for disease progression is about 0.3. In some embodiments, the hazard ratio for disease progression is about 0.4. In some embodiments, the hazard ratio for disease progression is about 0.45. In some embodiments, the hazard ratio for disease progression is about 0.5. In some embodiments, the hazard ratio for disease progression is less than about 0.5. In some embodiments, the hazard ratio for disease progression is less than about 0.45. In some embodiments, the hazard ratio for disease progression is less than about 0.4. In some embodiments, the hazard ratio for disease progression is less than about 0.35. In some embodiments, the hazard ratio for disease progression is less than about 0.3.

In some embodiments, the patient has at least (i) a germline mutation in BRCA1 or BRCA2 or (ii) a sporadic mutation in BRCA1 or BRCA2. In embodiments, the patient is further characterized by an absence of a germline mutation in BRCA1 or BRCA2. In embodiments, the patient is further characterized by an absence of a sporadic mutation in BRCA1 or BRCA2. In embodiments, the patient is further characterized by a negative BRCA1/2 status. In embodiments, a germline mutation in BRCA1 or BRCA2 is not detected in a sample from a patient. In embodiments, the population of subjects has a BRCA mutation. In embodiments, the BRCA mutation is a germline BRCA mutation (gBRCAmut). In embodiments, the BRCA mutation is a somatic (or sporadic) BRCA mutation (sBRCAmut). In embodiments, the population of subjects has a positive homologous recombination deficiency status. In embodiments, the population of subjects exhibits non-mutated BRCA1/2 “BRCAwt” or “BRCAwt”.

In some embodiments, the methods reduce the hazard ratio for disease progression or death as compared to control.

In embodiments, the patient is further characterized by an absence of a germline mutation in BRCA1 or BRCA2. In embodiments, the patient is further characterized by an absence of a sporadic mutation in BRCA1 or BRCA2. In embodiments, the patient is further characterized by a negative BRCA1/2 status. In embodiments, a germline mutation in BRCA1 or BRCA2 is not detected in a sample from a patient. In embodiments, the population of subjects exhibits non-mutated BRCA1/2 “BRCAwt” or “BRCAwt”.

In embodiments, the population of subjects has a BRCA mutation. In some embodiments, the patient also has at least (i) a germline mutation in BRCA1 or BRCA2 or (ii) a sporadic mutation in BRCA1 or BRCA2. In embodiments, the BRCA mutation is a germline BRCA mutation (gBRCAmut). In embodiments, the BRCA mutation is a somatic (or sporadic) BRCA mutation (sBRCAmut).

In some embodiments, the patient also has a germline mutation in BRCA1 and/or BRCA2 (gBRCAmut). In some embodiments, the prolonged progression free survival is at least 9-months. In some embodiments, the prolonged progression free survival is at least 10-months. In some embodiments, the prolonged progression free survival is at least 11-months. In some embodiments, the prolonged progression free survival is at least 12-months. In some embodiments, the prolonged progression free survival is at least 15-months. In some embodiments, the prolonged progression free survival is at least 18-months. In some embodiments, the prolonged progression free survival is at least 21-months. In some embodiments, the prolonged progression free survival is at least 24-months. In some embodiments, the prolonged progression free survival is at least 27-months. In some embodiments, the prolonged progression free survival is at least 30-months. In some embodiments, the prolonged progression free survival is at least 33-months. In some embodiments, the prolonged progression free survival is at least-months.

In some embodiments, the patient is characterized by an absence of a mutation in BRCA1 and/or BRCA2 (BRCAwt). In some embodiments, the prolonged progression free survival is at least 3-months. In some embodiments, the prolonged progression free survival is at least 6-months. In some embodiments, the prolonged progression free survival is at least 9-months. In some embodiments, the prolonged progression free survival is at least 10-months. In some embodiments, the prolonged progression free survival is at least 11-months. In some embodiments, the prolonged progression free survival is at least 12-months. In some embodiments, the prolonged progression free survival is at least 15-months. In some embodiments, the prolonged progression free survival is at least 18-months. In some embodiments, the prolonged progression free survival is at least 21-months. In some embodiments, the prolonged progression free survival is at least 24-months. In some embodiments, the prolonged progression free survival is at least 27-months. In some embodiments, the prolonged progression free survival is at least 30-months. In some embodiments, the prolonged progression free survival is at least 33-months. In some embodiments, the prolonged progression free survival is at least 36-months.

Prolonged Overall Survival

In embodiments, methods described herein comprise administering a PARP inhibitor (e.g., niraparib) according to a regimen determined to achieve prolonged overall survival in a cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2). Alternatively, or in addition to, the cancer patient has a deficiency is in one or more of the genes TP3 and/or RB1. In embodiments, a non-BRCA1/2 HRR deficiency is in one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L, and optionally a deficiency in BRCA1 and/or BRCA2. Alternatively, or in addition to, the deficiency is in one or more of the genes TP3 and/or RB1. In embodiments, a non-BRCA1/2 HRR deficiency is in one or more, two or more, three or more, four or more, five or more, seven or more, eight or more, nine or more, ten or more, eleven or more genes, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2, and optionally a deficiency in BRCA1 and/or BRCA2. Alternatively, or in addition to, the deficiency is in one or more of the genes TP3 and/or RB1.

In some embodiments, the prolonged overall survival is greater in patients receiving a PARP inhibitor (e.g., niraparib), for example as compared with patients not receiving a PARP inhibitor (e.g., niraparib). In some embodiments, prolonged overall survival is greater in patients receiving niraparib than in patients receiving alternative cancer therapy (e.g., patients receiving niraparib have a greater progression free survival than patients receiving therapy with a different PARP inhibitor). In embodiments, a patient has recurrent or platinum sensitive ovarian cancer, fallopian tube cancer, or primary peritoneal cancer. In embodiments, the patient has high grade serous ovarian cancer or high grade predominantly serous histology ovarian cancer. In embodiments, a patient has non-small cell lung cancer (NSCLC).

In some embodiments, the patient has at least (i) a germline mutation in BRCA1 or BRCA2 or (ii) a sporadic mutation in BRCA1 or BRCA2. In embodiments, the patient is further characterized by an absence of a germline mutation in BRCA1 or BRCA2. In embodiments, the patient is further characterized by an absence of a sporadic mutation in BRCA1 or BRCA2. In embodiments, the patient is further characterized by a negative BRCA1/2 status. In embodiments, a germline mutation in BRCA1 or BRCA2 is not detected in a sample from a patient. In embodiments, the population of subjects has a BRCA mutation. In embodiments, the BRCA mutation is a germline BRCA mutation (gBRCAmut). In embodiments, the BRCA mutation is a somatic (or sporadic) BRCA mutation (sBRCAmut). In embodiments, the population of subjects has a positive homologous recombination deficiency status. In embodiments, the population of subjects exhibits non-mutated BRCA1/2 “BRCAwt” or “BRCAwt”.

In some embodiments, the methods prolong overall survival as compared to control.

In embodiments, the patient is further characterized by an absence of a germline mutation in BRCA1 or BRCA2. In embodiments, the patient is further characterized by an absence of a sporadic mutation in BRCA1 or BRCA2. In embodiments, the patient is further characterized by a negative BRCA1/2 status. In embodiments, a germline mutation in BRCA1 or BRCA2 is not detected in a sample from a patient. In embodiments, the population of subjects exhibits non-mutated BRCA1/2 “BRCAwt” or “BRCAwt”.

In embodiments, the population of subjects has a BRCA mutation. In some embodiments, the patient also has at least (i) a germline mutation in BRCA1 or BRCA2 or (ii) a sporadic mutation in BRCA1 or BRCA2. In embodiments, the BRCA mutation is a germline BRCA mutation (gBRCAmut). In embodiments, the BRCA mutation is a somatic (or sporadic) BRCA mutation (sBRCAmut).

In some embodiments, the patient also has a germline mutation in BRCA1 and/or BRCA2 (gBRCAmut). In some embodiments, the prolonged progression free survival is at least 9-months. In some embodiments, the prolonged progression free survival is at least 10-months. In some embodiments, the prolonged progression free survival is at least 11 months. In some embodiments, the prolonged progression free survival is at least 12-months. In some embodiments, the prolonged progression free survival is at least 15-months. In some embodiments, the prolonged progression free survival is at least 18-months. In some embodiments, the prolonged progression free survival is at least 21-months. In some embodiments, the prolonged progression free survival is at least 24-months. In some embodiments, the prolonged progression free survival is at least 27-months. In some embodiments, the prolonged progression free survival is at least 30-months. In some embodiments, the prolonged progression free survival is at least 33-months. In some embodiments, the prolonged progression free survival is at least 36-months.

In some embodiments, the patient is characterized by an absence of a mutation in BRCA1 and/or BRCA2 (BRCAwt). In some embodiments, the prolonged progression free survival is at least 3-months. In some embodiments, the prolonged progression free survival is at least 6 months. In some embodiments, the prolonged progression free survival is at least 9-months. In some embodiments, the prolonged progression free survival is at least 10-months. In some embodiments, the prolonged progression free survival is at least 11-months. In some embodiments, the prolonged progression free survival is at least 12-months. In some embodiments, the prolonged progression free survival is at least 15-months. In some embodiments, the prolonged progression free survival is at least 18-months. In some embodiments, the prolonged progression free survival is at least 21-months. In some embodiments, the prolonged progression free survival is at least 24-months. In some embodiments, the prolonged progression free survival is at least 27-months. In some embodiments, the prolonged progression free survival is at least 30-months. In some embodiments, the prolonged progression free survival is at least 33-months. In some embodiments, the prolonged progression free survival is at least 36-months.

Additional Features

In some embodiments, methods described herein achieve an overall response rate of at least 30%. In some embodiments, methods described herein achieve improved progression free survival 2 as compared to control. In some embodiments, methods described herein achieve improved chemotherapy free interval as compared to control. In some embodiments, methods described herein achieve improved time to first subsequent therapy as compared to control. In some embodiments, methods described herein achieve improved time to second subsequent therapy as compared to control. In some embodiments, methods described herein have been determined to not have a detrimental effect on Quality of Life as determined by FOSI and/or EQ-5D-5L. In some embodiments, methods described herein have been determined to not impact the effectiveness of a subsequent treatment with another therapeutic agent (e.g., a chemotherapeutic agent such as a platinum agent, including but not limited to, cisplatin, carboplatin, oxaliplatin, nedaplatin, triplatin tetranitrate, phenanthriplatin, picoplatin, or satraplatin; or an immune checkpoint inhibitor (e.g., an agent that inhibits programmed death-1 protein (PD-1) signaling, T-cell immunoglobulin domain and mucin domain 3 (TIM-3), cytotoxic T-lymphocyte-associated protein 4 (CTLA-4), lymphocyte activation gene-3 (LAG-3), or T cell immunoglobulin and ITIM domain (TIGIT)).

Measuring Tumor Response

Tumor response can be measured by, for example, the RECIST v 1.1 guidelines. The guidelines are provided by E. A. Eisenhauer, et al., “New response evaluation criteria in solid tumors: Revised RECIST guideline (version 1.1.)”, Eur. J. of Cancer, 45: 228-47 (2009), which is incorporated by reference in its entirety. The guidelines require, first, estimation of the overall tumor burden at baseline, which is used as a comparator for subsequent measurements. Tumors can be measured via use of any imaging system known in the art, for example, by a CT scan, or an X-ray. Magnetic resonance imaging (MRI) may be used, for example, when CT is contradicted or for imaging of the brain. In some embodiments, CT imaging is the preferred imaging technique. In some embodiments, the same imaging technique is used for the patient throughout the entire study. Measurable disease is defined by the presence of at least one measurable lesion. In studies where the primary endpoint is tumor progression (either time to progression or proportion with progression at a fixed date), the protocol must specify if entry is restricted to those with measurable disease or whether patients having non-measurable disease only are also eligible.

In some embodiments, measurable disease is defined by the presence of at least one measurable lesion. When more than one measurable lesion is present at baseline, all lesions up to a maximum of five lesions total (and a maximum of two lesions per organ) representative of all involved organs should be identified as target lesions and will be recorded and measured at baseline (this means in instances where patients have only one or two organ sites involved a maximum of two and four lesions respectively will be recorded).

Target lesions should be selected on the basis of their size (lesions with the longest diameter), be representative of all involved organs, but in addition should be those that lend themselves to reproducible repeated measurements.

Lymph nodes merit special mention since they are normal anatomical structures which may be visible by imaging even if not involved by tumor. Pathological nodes which are defined as measurable and may be identified as target lesions must meet the criterion of a short axis of P15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT scan this is almost always the axial plane; for MRI the plane of acquisition may be axial, saggital or coronal). The smaller of these measures is the short axis.

For example, an abdominal node which is reported as having a short axis of 20 mm and qualifies as a malignant, measurable node. In this example, 20 mm should be recorded as the node measurement. All other pathological nodes (those with short axis P10 mm but <15 mm) should be considered non-target lesions. Nodes that have a short axis <10 mm are considered non-pathological and should not be recorded or followed.

A sum of the diameters (longest for non-nodal lesions, short axis for nodal lesions) for all target lesions will be calculated and reported as the baseline sum diameters. If lymph nodes are to be included in the sum, then as noted above, only the short axis is added into the sum. The baseline sum diameters will be used as reference to further characterize any objective tumor regression in the measurable dimension of the disease.

All other lesions (or sites of disease) including pathological lymph nodes should be identified as non-target lesions and should also be recorded at baseline. Measurements are not required and these lesions should be followed as “present”, “absent”, or in rare cases “unequivocal progression”. In addition, it is possible to record multiple nontarget lesions involving the same organ as a single item on the case record form (e.g. ‘multiple enlarged pelvic lymph nodes’ or ‘multiple liver metastases’).

In some embodiments, the first on-study imaging assessment should be performed at 9-weeks (63 days 7 days) from the date of the first dose of the study treatment. In some embodiments, in the case of progressive disease (PD), a confirmatory image will be required 4-weeks later (91 days 7 days).

In some embodiments, subsequent imaging should be performed every 9 weeks (63 days 7 days) or more frequently if clinically indicated at the time of suspected disease progression.

In some embodiments, after 1 year of radiographic assessments, patients will have imaging performed every 12-weeks (84 days 7 days).

In some embodiments, imaging will continue to be performed until one of the following occurs: the start of a new cancer treatment, the patient withdrawals consent, the patient dies, or the end of the study has been reached.

In some embodiments, patients who discontinue study treatment for reasons other than PD, will continue post-treatment imaging studies for disease status follow-up every 9-weeks (63 days 7 days) depending on the length of treatment with the study until: disease progression, the patient starts a new treatment outside of the study, the patient withdrawals consent, the patient becomes lost to follow-up, the patient dies, or the end of the study has been reached.

In some embodiments, irRECIST guidelines will also be incorporated in cases of disease progression to account for unique tumor characteristics seen during treatment with pembrolizumab and to assess continuation of treatment in clinically stable patients until progression is confirmed. In some embodiments, RECIST v1.1 is adapted to incorporate these special guidelines, as using RECIST v1.1 alone in immunotherapy trials would lead to the declaration of progressive disease (PD) too early. Antibody agents that inhibit PD-1 signaling (e.g., pembrolizumab) may produce antitumor effects by potentiating endogenous cancer-specific immune responses. The response patterns with this type of approach tend to extend beyond the typical time course of responses seen with cytotoxic agents and can manifest a clinical response after an initial increase in tumor burden or appearance of new lesions.

Therefore, in some embodiments if repeat imaging shows <20% increase in tumor burden compared with (1) nadir, stable, or improved previously indicated new lesion (if identified as cause for initial PD), and (2) stable/improved non-target disease (if identified as cause for initial PD), treatment may be continued or resumed, and the next imaging should be conducted according to the above protocol schedule of 9-weeks (63 days 7 days) or if it has been one year since beginning of treatment (first radiographic image taken), 12 weeks (84 days 7 days).

In some embodiments, incorporating both RECIST v1.1 plus irRESIST v1.1 guidelines, patients will be discontinued from the study if repeat imaging confirms PD due to any of the following: tumor burden remains ≥20% and at least a 5 mm absolute increase in tumor size compared with nadir, non-target disease resulting in initial PD is worse, new lesion resulting in initial PD is worse, additional new lesions appeared since last evaluation, additional new non-target progression is seen since last evaluation.

In some embodiments, incorporating both RECIST v1.1 plus irRESIST v1.1 guidelines, patients may remain on pembrolizumab while waiting for confirmation of PD if they are clinically stable, which means the patient has absence of signs and symptoms indicating clinically significant progression of disease including worsening of laboratory values, the patient has no decline in ECOG status (0=asymptomatic through 5=death), patient is absent of rapid progression of disease, and patient has absence of progressive tumor at critical anatomical sites. Patients on immunotherapy can have transient tumor flare in the first few months of treatment, but with subsequent disease response. Thus, it is best to keep patients on the treatment while waiting for confirmation of PD if possible.

In some embodiments, the primary efficacy endpoint for the study is objective response rate (ORR) defined as a proportion of patients achieving CR or PR as assessed by RECIST v1.1. ORR by irRESIST will also be evaluated as a secondary endpoint. Tumor assessments after the initiation of further anticancer therapy are excluded for assessment of best overall response.

In some embodiments, duration of response (DOR) will be evaluated as a secondary endpoint. In some embodiments, DOR is defined as the time from first documentation of CR or PR by RESIST v1.1 guidelines until (1) the time of first documentation of disease progression per RESIST v1.1 and (2) the time of first documentation of disease progression per irRESIST. In some embodiments, date of progression based on RESIST v1.1 or irRESIST may be overwritten in patients with OC if clinical criteria indicate earlier progression as adjucated by the study committee.

In some embodiments, disease control rate (DCR) will be assessed as a secondary endpoint and is defined as the proportion of patients achieving CR, PR, or SD as assessed by RESIST v1.1 and irRESIST.

In some embodiments, progression-free survival (PFS) will be assessed as secondary endpoint and is defined as the time from enrollment to the earlier date of assessment of progression or death by any cause in the absence of progression based on (1) the time of first documentation of disease progression per RESIST v1.1 and (2) the time of first documentation of disease progression per irRESIST. In some embodiments, date of progression based on RESIST v1.1 or irRESIST may be overwritten in patients with OC if clinical criteria indicate earlier progression as adjucated by the study committee.

In some embodiments, overall survival (OS) will be assessed as a secondary endpoint and is defined as the time from date of first dose of study treatment to the date of death by any cause. New malignancy information will also be collected as part of this assessment.

In some embodiments, tumor markers (CA-125) will not be used for defining objective responses or disease progression, but can be used for clinical decisions.

In some embodiments, clinical criteria GCIG will be used for management of OC patients with clinical events (e.g., niraparib bowel obstruction) without radiographic evidence of disease progression.

Dosage and Dosage Regimens

As described herein, provided methods comprise administering a PARP inhibitor such as niraparib to a cancer patient having a non-BRCA1/2 HRR deficiency as described herein (e.g., an identified deficiency in one or more, two or more, three or more, four or more, five or more, seven or more, eight or more, nine or more, ten or more, eleven or more, twelve or more, thirteen or more, fourteen or more, or fifteen or more genes selected from the group consisting of ATM, ATR, BAP1, BARD1, BLM, BRIP1, MRE11A, NBN, PALB2, RAD51, RAD51B, RAD51C, RAD51D, RAD52, RAD54L, and XRCC2 and optionally an identified deficiency in BRCA1 and/or BRCA2). Alternatively, or in addition to, the cancer patient has a deficiency is in one or more of the genes TP3 and/or RB1. In embodiments, a non-BRCA1/2 HRR deficiency is in one or more, two or more, three or more, four or more, five or more, seven or more, eight or more, nine or more, ten or more, or eleven or more genes selected from the group consisting of ATM, ATR, BARD1, BRIP1, MRE11A, NBN, PALB2, RAD51, RAD51B, RAD51C, RAD51D, and RAD54L, and optionally a deficiency in BRCA1 and/or BRCA2. Alternatively, or in addition to, the deficiency is in one or more of the genes TP3 and/or RB1. In embodiments, a non-BRCA1/2 HRR deficiency is in one or more, two or more, three or more, four or more, five or more, seven or more, eight or more, nine or more, ten or more, eleven or more genes, twelve or more, thirteen or more, or fourteen or more genes selected from the group consisting of ATM, ATR, BAP1, BARD1, BRIP1, MRE11A, NBN, PALB2, RAD51, RAD51B, RAD51C, RAD51D, RAD52, RAD54L, and XRCC2, and optionally a deficiency in BRCA1 and/or BRCA2. Alternatively, or in addition to, the deficiency is in one or more of the genes TP3 and/or RB1.

In embodiments, the administration is according to a regimen that achieves any one of or combination of: prolonged progression free survival; reduced hazard ratio for disease progression or death; and/or prolonged overall survival or a positive overall response rate (e.g., a regimen as described herein).

In embodiments, a PARP inhibitor (e.g., niraparib) is administered to a patient or population of subjects who has exhibited response to prior therapy. In embodiments, the patient or population of subjects has exhibited response to prior therapy with a chemotherapeutic agent. In embodiments, the chemotherapeutic agent is a platinum agent.

In embodiments, a PARP inhibitor (e.g., niraparib) is administered as a maintenance therapy following complete or partial response to at least one platinum based therapy or at least two platinum-based therapies. In embodiments, a platinum-based therapy comprises administering to a patient in need thereof a platinum-based agent selected from cisplatin, carboplatin, oxaliplatin, nedaplatin, triplatin tetranitrate, phenanthriplatin, picoplatin, or satraplatin. In embodiments, response to the most recent platinum-based chemotherapy regimen is a complete response. In embodiments, response to the most recent platinum-based chemotherapy regimen is a partial response. In embodiments, response to the penultimate platinum-based chemotherapy regimen is a complete response. In some embodiments, response to the penultimate platinum-based chemotherapy regimen is a partial response.

In embodiments, a PARP inhibitor is niraparib. In embodiments, a patient is administered a dose equivalent to about 100 mg, about 200 mg, about 300 mg, about 400 mg, or about 500 mg of niraparib, or a salt or derivative thereof (e.g., a dose equivalent to about 100 mg, about 200 mg, or about 300 mg of niraparib free base). In embodiments, administered niraparib comprises niraparib tosylate monohydrate. In embodiments, administered niraparib is administered as niraparib tosylate monohydrate.

In embodiments, niraparib is administered at a dose equivalent to about 100 mg of niraparib free base (e.g., a pharmaceutically acceptable salt of niraparib such as niraparib tosylate monohydrate is administered at a dose equivalent to about 100 mg of niraparib free base). In embodiments, niraparib is administered at a dose equivalent to about 200 mg of niraparib free base (e.g., a pharmaceutically acceptable salt of niraparib such as niraparib tosylate monohydrate is administered at a dose equivalent to about 200 mg of niraparib free base. In embodiments, niraparib is administered at a dose equivalent to about 300 mg of niraparib free base (e.g., a pharmaceutically acceptable salt of niraparib such as niraparib tosylate monohydrate is administered at a dose equivalent to about 300 mg of niraparib free base).

In embodiments, an administered amount of niraparib is about 300 mg of niraparib (e.g., an amount of a pharmaceutically acceptable salt of niraparib such as niraparib tosylate monohydrate equivalent to about 300 mg of niraparib free base). In some embodiments, the regimen comprises administration of 300 mg of niraparib once daily (e.g., an amount of a pharmaceutically acceptable salt of niraparib such as niraparib tosylate monohydrate equivalent to about 300 mg of niraparib free base once daily).

In some embodiments, an administered amount of niraparib is about 200 mg of niraparib (e e.g., an amount of a pharmaceutically acceptable salt of niraparib such as niraparib tosylate monohydrate equivalent to about 200 mg of niraparib free base). In some embodiments, the regimen comprises administration of 200 mg of niraparib once daily (e.g., an amount of a pharmaceutically acceptable salt of niraparib such as niraparib tosylate monohydrate equivalent to about 200 mg of niraparib free base once daily).

In some embodiments, an administered amount of niraparib is about 100 mg of niraparib (e.g., an amount of a pharmaceutically acceptable salt of niraparib such as niraparib tosylate monohydrate equivalent to about 100 mg of niraparib free base). In some embodiments, the regimen comprises administration of 100 mg of niraparib once daily (e.g., an amount of a pharmaceutically acceptable salt of niraparib such as niraparib tosylate monohydrate equivalent to about 100 mg of niraparib free base once daily).

In some embodiments, the regimen comprises at least one 21-day cycle. In some embodiments, the regimen comprises a plurality of 21-day cycles. In some embodiments, the regimen comprises one 21-day cycle. In some embodiments, the regimen comprises two 21-day cycles. In some embodiments, the regimen comprises three 21-day cycles. In some embodiments, the regimen comprises continuous 21 day cycles. In some embodiments, the regimen comprises administration of an effective dose of a PARP inhibitor such as niraparib daily until disease progression or unacceptable toxicity occurs. In some embodiments, the regimen comprises a daily dose of at least about 100, 200, or 300 mg niraparib per day dosed until disease progression or unacceptable toxicity occurs (e.g., a dose of a pharmaceutically acceptable salt of niraparib such as niraparib toslyate monohydrate in an amount equivalent to at least about 100, 200, or 300 mg niraparib free base or a dose of a pharmaceutically acceptable salt of niraparib such as niraparib toslyate monohydrate in an amount equivalent to about 100, 200, or 300 mg niraparib free base).

In some embodiments, the regimen comprises at least one 28-day cycle. In some embodiments, the regimen comprises a plurality of 28-day cycles. In some embodiments, the regimen comprises one 28-day cycle. In some embodiments, the regimen comprises two 28-day cycles. In some embodiments, the regimen comprises three 28-day cycles. In some embodiments, the regimen comprises continuous 28-day cycles. In some embodiments, the regimen comprises administration of an effective dose of a PARP inhibitor such as niraparib daily until disease progression or unacceptable toxicity occurs. In some embodiments, the regimen comprises a daily dose of at least 100, 200, or 300 mg niraparib per day dosed until disease progression or unacceptable toxicity occurs (e.g., a dose of a pharmaceutically acceptable salt of niraparib such as niraparib tosylate monohydrate in an amount equivalent to at least about 100, 200, or 300 mg niraparib free base or a dose of a pharmaceutically acceptable salt of niraparib such as niraparib tosylate monohydrate in an amount equivalent to about 100, 200, or 300 mg niraparib free base).

In some embodiments, a PARP inhibitor (e.g., niraparib) is administered in a regimen determined to achieve i) prolonged progression free survival as compared to control, ii) a reduced hazard ratio for disease progression or death as compared to control, iii) prolonged overall survival as compared to control, or iv) an overall response rate of at least 30%. In embodiments, a regimen comprises a daily dose (e.g., a daily oral dose) of niraparib (e.g., a daily oral dose of a pharmaceutically acceptable salt of niraparib such as niraparib tosylate monohydrate in an amount equivalent to about 200 mg or about 300 mg niraparib free base).

In some embodiments, the methods prolong progression free survival as compared to control. In some embodiments, the methods reduce the hazard ratio for disease progression or death as compared to control. In some embodiments, the methods prolong overall survival as compared to control. In some embodiments, the methods achieve an overall response rate of at least 30%. In some embodiments, the methods achieve improved progression free survival 2 as compared to control. In some embodiments, the methods achieve improved chemotherapy free interval as compared to control. In some embodiments, the methods achieve improved time to first subsequent therapy as compared to control. In some embodiments, the methods achieve improved time to second subsequent therapy as compared to control. In some embodiments, the methods have been determined to not have a detrimental effect on Quality of Life as determined by FOSI and/or EQ-5D-5L. In some embodiments, the methods have been determined to not impact the effectiveness of a subsequent treatment with a chemotherapeutic agent (e.g., a platinum agent, including but not limited to, cisplatin, carboplatin, oxaliplatin, nedaplatin, triplatin tetranitrate, phenanthriplatin, picoplatin, or satraplatin.

Oral Dosage Regimens

In some embodiments, the regimen comprises at least one oral dose of a PARP inhibitor such as niraparib. In some embodiments, the regimen comprises a plurality of oral doses. In some embodiments, the regimen comprises once daily (QD) dosing. In embodiments, a regimen comprises a once daily dose of a pharmaceutically acceptable salt of niraparib such as niraparib tosylate monohydrate in an amount equivalent to about 200 mg or about 300 mg niraparib free base.

In some embodiments, the oral dose is an amount of a PARP inhibitor (e.g., niraparib) within a range of about 10 mg to about 500 mg. In some embodiments, the dose is within a range of about 25 mg to about 400 mg. In some embodiments, the dose is within a range of about 50 mg to about 300 mg. In some embodiments, the dose is within a range of about 150 mg to about 350 mg. In some embodiments, the dose is within a range of about 50 mg to about 250 mg. In some embodiments, the dose is within a range of about 50 mg to about 200 mg. In some embodiments, the dose is within a range of about 50 mg to about 100 mg. In some embodiments, the dose is within a range of about 100 mg to about 300 mg. In embodiments, a PARP inhibitor is niraparib.

In some embodiments, the oral dose is an amount of a PARP inhibitor (e.g., niraparib) within a range of about 10 mg to about 500 mg. In some embodiments, the dose is within a range of about 25 mg to about 400 mg. In some embodiments, the dose is within a range of about 50 mg to about 300 mg. In some embodiments, the dose is within a range of about 150 mg to about 350 mg. In some embodiments, the dose is within a range of about 50 mg to about 250 mg. In some embodiments, the dose is within a range of about 50 mg to about 200 mg. In some embodiments, the dose is within a range of about 50 mg to about 100 mg. In some embodiments, the dose is within a range of about 100 mg to about 300 mg. In embodiments, a PARP inhibitor is niraparib.

In some embodiments, the oral dose is an amount of niraparib within a range of about 5 to about 400 mg (an amount equivalent to about 5 to about 400 mg of niraparib free base). In some embodiments, the amount of niraparib is about 5, about 10, about 25, about 50, about 100, about 150, about 200, about 250, about 300, about 350, or about 400 mg (e.g., an amount equivalent to about 5, about 10, about 25, about 50, about 100, about 150, about 200, about 250, about 300, about 350, or about 400 mg of niraparib free base). In embodiments, an oral dose comprises niraparib tosylate monohydrate.

In embodiments, an oral dose comprises niraparib (e.g., a pharmaceutically acceptable salt of niraparib such as niraparib tosylate monohydrate) in an amount equivalent to about 5 to about 400 mg of niraparib free base. In embodiments, an oral dose comprises niraparib (e.g., a pharmaceutically acceptable salt of niraparib such as niraparib tosylate monohydrate) in an amount equivalent to about 5 to about 400 mg of niraparib free base. In embodiments, an oral dose comprises an amount of niraparib (e.g., a pharmaceutically acceptable salt of niraparib such as niraparib tosylate monohydrate) that is equivalent to about 5, about 10, about 25, about 50, about 100, about 150, about 200, about 250, about 300, about 350, or about 400 mg of niraparib free base.

In some embodiments, an oral dose comprises niraparib (e.g., a pharmaceutically acceptable salt of niraparib such as niraparib tosylate monohydrate) in an amount equivalent to about 300 mg of niraparib free base. In some embodiments, the regimen comprises oral administration of niraparib (e.g., a pharmaceutically acceptable salt of niraparib such as niraparib tosylate monohydrate) in an amount equivalent to about 300 mg of niraparib free base once daily.

In some embodiments, an oral dose comprises niraparib (e.g., a pharmaceutically acceptable salt of niraparib such as niraparib tosylate monohydrate) in an amount equivalent to about 200 mg of niraparib free base. In some embodiments, the regimen comprises oral administration of niraparib (e.g., a pharmaceutically acceptable salt of niraparib such as niraparib tosylate monohydrate) in an amount equivalent to about 200 mg of niraparib free base once daily.

In some embodiments, an oral dose comprises niraparib (e.g., a pharmaceutically acceptable salt of niraparib such as niraparib tosylate monohydrate) in an amount equivalent to about 100 mg of niraparib free base. In some embodiments, the regimen comprises oral administration of niraparib (e.g., a pharmaceutically acceptable salt of niraparib such as niraparib tosylate monohydrate) in an amount equivalent to about 100 mg of niraparib free base once daily.

Formulations

In some embodiments, the oral dose is administered in one or more unit dosage forms. In some embodiments, the one or more unit dosage forms are capsules. In some embodiments, the one or more unit dosage forms are tablets.

In embodiments, each unit dosage form comprises about 5, about 10, about 25, about 50, or about 100 mg of niraparib. In embodiments, each unit dosage form comprises an amount equivalent to about 5, about 10, about 25, about 50, or about 100 mg of niraparib free base (e.g., each unit dosage form comprises a pharmaceutically acceptable salt of niraparib such as niraparib tosylate monohydrate in an amount equivalent to about 5, about 10, about 25, about 50, or about 100 mg of niraparib free base).

In embodiments, a 100 mg unit dosage form comprises niraparib (e.g., a pharmaceutically acceptable salt of niraparib such as niraparib tosylate monohydrate) in an amount equivalent to about 100 mg of niraparib free base. In embodiments, a unit dosage form is a tablet. In embodiments, a unit dosage form is a capsule.

It is understood that any combination of unit dosage forms can be combined to form a once daily (QD) dose. For example, three 100 mg unit dosage forms (e.g., each unit dosage form comprising an amount of niraparib—such as a pharmaceutically acceptable salt of niraparib that is niraparib tosylate monohydrate—that is equivalent to about 100 mg of niraparib free base) can be taken once daily such that about 300 mg of niraparib (e.g., about 300 mg of niraparib free base) is administered once daily, or two 100 mg unit dosage forms (e.g., each unit dosage form comprising an amount of niraparib—such as a pharmaceutically acceptable salt of niraparib that is niraparib tosylate monohydrate—that is equivalent to about 100 mg of niraparib free base) can be taken once daily such that about 200 mg of niraparib (e.g., about 200 mg of niraparib free base) is administered once daily.

In some embodiments, niraparib is administered as a single 100 mg unit dosage form (e.g., a single unit dosage form comprising niraparib (e.g., a pharmaceutically acceptable salt of niraparib such as niraparib tosylate monohydrate) in an amount equivalent to about 100 mg niraparib free base). In some embodiments, niraparib is administered 100 mg QD; for example, an amount of niraparib (e.g., a pharmaceutically acceptable salt of niraparib such as niraparib tosylate monohydrate) that is equivalent to about 100 mg niraparib free base.

In some embodiments, niraparib is administered as a single 200 mg unit dosage form (e.g., a single unit dosage form comprising niraparib (e.g., a pharmaceutically acceptable salt of niraparib such as niraparib tosylate monohydrate) in an amount equivalent to about 200 mg niraparib free base). In some embodiments, niraparib is administered 200 mg QD; for example, an amount of niraparib (e.g., a pharmaceutically acceptable salt of niraparib such as niraparib tosylate monohydrate) that is equivalent to about 200 mg niraparib free base. In some embodiments, niraparib is administered as 2×100 mg QD (i.e., niraparib is administered as two 100 mg unit dosage forms); for example, niraparib is administered as two unit dosage forms, each unit dosage form comprising niraparib (e.g., a pharmaceutically acceptable salt of niraparib such as niraparib tosylate monohydrate) in an amount equivalent to about 100 mg niraparib free base.

In some embodiments, niraparib is administered as a single 300 mg unit dosage form (e.g., a single unit dosage form comprising niraparib (e.g., a pharmaceutically acceptable salt of niraparib that is niraparib tosylate monohydrate) in an amount equivalent to about 300 mg niraparib free base). In some embodiments, niraparib is administered about 300 mg QD (e.g., an amount of a pharmaceutically acceptable salt of niraparib that is niraparib tosylate monohydrate that is equivalent to about 300 mg niraparib free base). In some embodiments, niraparib is administered as 3×100 mg QD (i.e., niraparib is administered as three unit dosage forms of about 100 mg); for example, niraparib is administered as three unit dosage forms, each unit dosage form comprising a pharmaceutically acceptable salt of niraparib (e.g., niraparib tosylate monohydrate) in an amount equivalent to about 100 mg niraparib free base. In some embodiments, niraparib is administered as 2×150 mg QD (i.e., niraparib is administered as two unit dosage forms of about 150 mg); for example, niraparib is administered as two unit dosage forms, each unit dosage form comprising a pharmaceutically acceptable salt of niraparib (e.g., niraparib tosylate monohydrate) in an amount equivalent to about 150 mg niraparib free base.

In some embodiments, the regimen comprises administration of an effective dose of a PARP inhibitor (e.g., niraparib) daily until disease progression or unacceptable toxicity occurs. In some embodiments, the regimen comprises a daily dose of 100 mg, 200 mg, 300 mg or more of a PARP inhibitor (e.g., niraparib) per day dosed until disease progression or unacceptable toxicity occurs. In some embodiments, the regimen comprises a daily dose of 300 mg of niraparib (e.g., a pharmaceutically acceptable salt of niraparib such as niraparib tosylate monohydrate) per day dosed until disease progression or unacceptable toxicity occurs. In some embodiments, the regimen comprises a daily dose of 200 mg of niraparib (e.g., a pharmaceutically acceptable salt of niraparib such as niraparib tosylate monohydrate) per day dosed until disease progression or unacceptable toxicity occurs. In some embodiments, the regimen comprises a daily dose of 100 mg of niraparib (e.g., a pharmaceutically acceptable salt of niraparib such as niraparib tosylate monohydrate) per day dosed until disease progression or unacceptable toxicity occurs.

In some embodiments, the range of an oral dose is bounded by a lower limit and an upper limit, the upper limit being larger than the lower limit.

In some embodiments, the lower limit may be about 10 mg, about 25 mg, about 50 mg, or about 100 mg of a PARP inhibitor (e.g., niraparib). In embodiments, the lower limit may be an amount of niraparib (e.g., a pharmaceutically acceptable salt of niraparib such as niraparib tosylate monohydrate) that is equivalent to about 10 mg, about 25 mg, about 50 mg, or about 100 mg of niraparib free base.

In some embodiments, the upper limit may be about 150 mg, about 200 mg, about 250 mg, about 300 mg, about 350 mg, about 400 mg or about 500 mg of a PARP inhibitor (e.g., niraparib). In embodiments, the upper limit may be an amount of niraparib (e.g., a pharmaceutically acceptable salt of niraparib such as niraparib tosylate monohydrate) that is equivalent to about 150 mg, about 200 mg, about 250 mg, about 300 mg, about 350 mg, about 400 mg or about 500 mg of niraparib free base.

Pharmacokinetics

Pharmacokinetic data can be obtained by known techniques in the art. Due to the inherent variation in pharmacokinetic and pharmacodynamic parameters of drug metabolism in human subjects, appropriate pharmacokinetic and pharmacodynamic profile components describing a particular composition can vary. Typically, pharmacokinetic and pharmacodynamic profiles are based on the determination of the mean parameters of a group of subjects. The group of subjects includes any reasonable number of subjects suitable for determining a representative mean, for example, 5-subjects, 10-subjects, 16-subjects, 20-subjects, 25-subjects, 30-subjects, 35-subjects, or more. The mean is determined by calculating the average of all subject's measurements for each parameter measured.

In some embodiments, the pharmacokinetic parameter(s) can be any parameters suitable for describing the present composition. For example, in some embodiments, the Cmax is not less than about 500 ng/ml; not less than about 550 ng/ml; not less than about 600 ng/ml; not less than about 700 ng/ml; not less than about 800 ng/ml; not less than about 880 ng/ml, not less than about 900 ng/ml; not less than about 100 ng/ml; not less than about 1250 ng/ml; not less than about 1500 ng/ml, not less than about 1700 ng/ml, or any other Cmax appropriate for describing a pharmacokinetic profile of the PARP inhibitor (e.g., niraparib).

In some embodiments wherein the active metabolite is formed in vivo after administration of a drug to a subject, the Cmax is not less than about 500 pg/ml; not less than about 550 pg/ml; not less than about 600 pg/ml; not less than about 700 pg/ml; not less than about 800 pg/ml; not less than about 880 pg/ml, not less than about 900 pg/ml; not less than about 1000 pg/ml; not less than about 1250 pg/ml; not less than about 1500 pg/ml, not less than about 1700 pg/ml, or any other Cmax appropriate for describing a pharmacokinetic profile of a compound formed in vivo after administration of the PARP inhibitor (e.g., niraparib) to a subject.

In some embodiments, the Tmax is, for example, not greater than about 0.5-hours, not greater than about 1.0-hours, not greater than about 1.5-hours, not greater than about 2.0-hours, not greater than about 2.5-hours, or not greater than about 3.0-hours, or any other Tmax appropriate for describing a pharmacokinetic profile of the PARP inhibitor (e.g., niraparib).

In general, AUC as described herein is the measure of the area under the curve that corresponds to the concentration of an analyte over a selected time period following administration of a dose of a therapeutic agent. In some embodiments, such time period begins at the dose administration (i.e., 0-hours after dose administration) and extends for about 2-hours, about 3-hours, about 4-hours, about 5-hours, about 6-hours, about 7-hours, about 8-hours, about 9-hours, about 10-hours, about 11-hours, about 12-hours, about 14-hours, about 16-hours, about 18-hours, about 20-hours, about 22-hours, about 24-hours, about 30-hours, about 40-hours, or more hours after the dose administration. In some embodiments, AUC is that achieved from 0-hours to 12-hours following administration of a dose described herein. In some embodiments, AUC is that achieved from 0-hours to 18-hours following administration of a dose described herein. In some embodiments, AUC is that achieved from 0 hours to 24 hours following administration of a dose described herein. In some embodiments, AUC is that achieved from 0 hours to 36 hours following administration of a dose described herein.

The AUC(0-inf) can be, for example, not less than about 590 ng·hr/mL, not less than about 1500 ng·hr/mL, not less than about 2000 ng·hr/mL, not less than about 3000 ng·times·hr/ml, not less than about 3500 ng·hr/mL, not less than about 4000 ng·hr/mL, not less than about 5000 ng·hr/mL, not less than about 6000 ng·hr/mL, not less than about 7000 ng·hr/mL, not less than about 8000 ng·hr/mL, not less than about 9000 ng·hr/mL, or any other AUCco-int) appropriate for describing a pharmacokinetic profile of a therapeutic agent (e.g., niraparib). In some embodiments wherein an active metabolite is formed in vivo after administration of a therapeutic agent (e.g., niraparib) to a subject; the AUC(0-inf) can be, for example, not less than about 590 pg·hr/mL, not less than about 1500 pg·hr/mL, not less than about 2000 pg·hr/mL, not less than about 3000 pg·hr/mL, not less than about 3500 pg·hr/mL, not less than about 4000 pg·hr/mL, not less than about 5000 pg·hr/mL, not less than about 6000 pg·hr/mL, not less than about 7000 pg·hr/mL, not less than about 8000 pg·hr/mL, not less than about 9000 pg·hr/mL, or any other AUC(0-inf) appropriate for describing a pharmacokinetic profile of a compound formed in vivo after administration of the PARP inhibitor (e.g., niraparib) to a subject.

The plasma concentration of niraparib about one hour after administration can be, for example, not less than about 140 ng/ml, not less than about 425 ng/ml, not less than about 550 ng/ml, not less than about 640 ng/ml, not less than about 720 ng/ml, not less than about 750 ng/ml, not less than about 800 ng/ml, not less than about 900 ng/ml, not less than about 1000 ng/ml, not less than about 1200 ng/ml, or any other plasma concentration of the PARP inhibitor (e.g., niraparib).

In some embodiments, a patient population includes one or more subjects (“a population of subjects”) suffering from metastatic disease.

In some embodiments, a patient population includes one or more subjects that are suffering from or susceptible to cancer. In some such embodiments, the cancer is ovarian cancer, cancer of the fallopian tubes, peritoneal cancer or breast cancer. In some embodiments, a patient population includes one or more subjects (e.g., comprises or consists of subjects) suffering from cancer. For example, in some embodiments, a patient population suffering from cancer may have previously been treated with chemotherapy, such as, e.g., treatment with a chemotherapeutic agent such as a platinum-based agent.

In some embodiments, the present disclosure provides methodologies that surprisingly can achieve substantially the same PK profile for the PARP inhibitor (e.g., niraparib) when administered to a patient in a fed state or in a fasted state. The PARP inhibitor (e.g., niraparib) can be administered to a patient in either a fed or fasted state. In some embodiments, administration of the PARP inhibitor (e.g., niraparib) to a patient in a fed or fasted state produces substantially bioequivalent PARP inhibitor (e.g., niraparib) plasma Cmax values. In some embodiments, administration to the patient in a fed or fasted state produces bioequivalent PARP inhibitor (e.g., niraparib) plasma Tmax values. In some embodiments, administration to the patient in a fed or fasted state produces bioequivalent PARP inhibitor (e.g., niraparib) plasma AUC values. Accordingly, in some embodiments, the PARP inhibitor (e.g., niraparib) is administered in either a fed or a fasted state. In some embodiments, the PARP inhibitor (e.g., niraparib) is administered in a fasted state. In another embodiment, the PARP inhibitor (e.g., niraparib) is administered in a fed state.

In some embodiments, a unit dose of the PARP inhibitor (e.g., niraparib) can be administered to a patient in a fasted state. In some embodiments, a unit dose of the PARP inhibitor (e.g., niraparib) can be administered to a patient in a fed state. In some embodiments, administration in one of the fed or fasted states is excluded. In some embodiments, the unit dose can be administered for therapeutic purposes in either the fed or the fasted state, with the subject having the option for each individual dose as to whether to take it with or without food. In some embodiments, the unit dose of the PARP inhibitor (e.g., niraparib) can be administered immediately prior to food intake (e.g., within 30 or within 60 minutes before), with food, right after food intake (e.g., within 30, 60 or 120 minutes after food intake). In some embodiments, it can be administered, for example, at least 2-hours, 3-hours, 4-hours, 5-hours, 6-hours, 7-hours, 8-hours, 9-hours, 10-hours, 11-hours, 12-hours, or more hours after food intake, or any time there between. In some embodiments, the unit dose of the PARP inhibitor (e.g., niraparib) is administered after overnight fasting. In some embodiments, the unit dose of the composition can be administered 30 minutes before food intake, 1 hour, 2 hours, 3 hours, 4 hours, 5 hours, 6 hours, 7 hours, 8 hours, 9 hours, 10 hours, 11 hours, 12 hours or more before food intake, or any time there between.

Combination Therapy

PARP inhibitors (e.g., niraparib) can be administered alone as a monotherapy or in combination with other therapies. Combination therapies that enhance or synergize with cytotoxic agents without significantly increasing toxicity would provide substantial benefit to ovarian as well other types of cancer patients.

In embodiments, a PARP inhibitor (e.g., niraparib) is administered in combination with at least one additional therapeutic agent or therapy. In embodiments, a PARP inhibitor such as niraparib is administered simultaneously or sequentially with an additional therapeutic agent, such as, for example, a chemotherapeutic agent. In some embodiments, a PARP inhibitor (e.g., niraparib) is administered before, during, or after administration of an additional therapeutic agent (e.g., a chemotherapeutic agent). In embodiments, administering of a PARP inhibitor (e.g., niraparib) and an at least one additional therapeutic agent is according to a regimen that achieves any one of or combination of: prolonged progression free survival; reduced hazard ratio for disease progression or death; and/or prolonged overall survival or a positive overall response rate. In embodiments, administering of a PARP inhibitor (e.g., niraparib) is according to any of the regimens described herein.

When administered as part of a combination therapy, a PARP inhibitor (e.g., niraparib) can be administered according to any of the regimens and formulations described herein. For example, the PARP inhibitor (e.g., niraparib) can be administered according to any of the oral dosing regimens described herein.

Administration of the PARP inhibitor (e.g., niraparib) can occur simultaneously or sequentially with an additional therapeutic agent (e.g., a chemotherapeutic agent). In embodiments, niraparib can be administered prior to (e.g., 5-minutes, 15-minutes, 30-minutes, 45-minutes, 1-hour, 2-hours, 4-hours, 6-hours, 12-hours, 24-hours, 48-hours, 72-hours, 96-hours, 1-week, 2-weeks, 3-weeks, 4-weeks, 5-weeks, 6-weeks, 8-weeks, or 12-weeks) before, concurrently with, or subsequent to (e.g., 5-minutes, 15-minutes, 30-minutes, 45-minutes, 1-hour, 2-hours, 4-hours, 6-hours, 12-hours, 24-hours, 48-hours, 72-hours, 96-hours, 1-week, 2-weeks, 3-weeks, 4-weeks, 5-weeks, 6-weeks, 8-weeks, or 12-weeks) after the administration of the chemotherapeutic agent to a subject in need thereof. In some embodiments the PARP inhibitor (e.g., niraparib) and the chemotherapeutic agent are administered 1-minute apart, 10-minutes apart, 30-minutes apart, less than 1-hour apart, 1-hour to 2-hours apart, 2-hours to 3-hours apart, 3-hours to 4-hours apart, 4-hours to 5-hours apart, 5-hours to 6-hours apart, 6-hours to 7-hours apart, 7-hours to 8-hours apart, 8-hours to 9-hours apart, 9-hours to 10-hours apart, 10-hours to 11-hours apart, 11-hours to 12-hours apart, no more than 24-hours apart, or no more than 48-hours apart.

Chemotherapeutic Agents

In embodiments, a PARP inhibitor (e.g., niraparib) is administered in combination (e.g., simultaneously or sequentially) with at least one additional chemotherapeutic (i.e., a chemical agent that inhibits the proliferation, growth, life-span and/or metastatic activity of cancer cells).

Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines (e.g., altretamine, triethylenemelamine, trietylenephosphoramide, triethiylenethiophosphoramide and trimethylolomelamine); acetogenins; delta-9-tetrahydrocannabinol (e.g., dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (e.g., cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulene Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; xeloda; ibandronate; topoisomerase inhibitor RFS 2000; difluoromethylornithine (DMFO); retinoids such as retinoic acid; capecitabine; pharmaceutically acceptable salts, acids or derivatives of any of the above; as well as combinations of two or more of the above such as CHOP, an abbreviation for a combined therapy of cyclophosphamide, doxorubicin, vincristine, and prednisone, and FOLFOX, an abbreviation for a treatment regimen with oxaliplatin (ELOXATIN™) combined with 5-FU and leucovovin.

Chemotherapeutic agents also include anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In embodiments, a PARP inhibitor (e.g., niraparib) is administered in combination with at least one additional therapeutic agent that is cisplatin, carboplatin, an alkylating (e.g., methylating) agent, or a topoisomerase I inhibitor. In embodiments, a PARP inhibitor (e.g., niraparib) is administered in combination with radiation therapy.

In embodiments, a PARP inhibitor such as niraparib is administered to a patient simultaneously or sequentially with a chemotherapeutic agent. In some embodiments, a PARP inhibitor (e.g., niraparib) is administered before, during, or after administration of a chemotherapeutic agent. In embodiments, a chemotherapeutic agent is a platinum chemotherapeutic agent (e.g., cisplatin, carboplatin, oxaliplatin, nedaplatin, triplatin tetranitrate, phenanthriplatin, picoplatin, or satraplatin). In embodiments, a patient has a gynecological cancer (e.g., any gynecological cancer as described herein).

Immune Checkpoint Inhibitors

In embodiments, a PARP inhibitor (e.g., niraparib) is administered in combination (e.g., simultaneously or sequentially) with at an immune checkpoint inhibitor. In embodiments, a cancer patient is suffering or is at risk of non-small cell lung cancer (NSCLC).

In embodiments, an immune checkpoint inhibitor is an agent that inhibits programmed death-1 protein (PD-1) signaling, T-cell immunoglobulin domain and mucin domain 3 (TIM-3), cytotoxic T-lymphocyte-associated protein 4 (CTLA-4), lymphocyte activation gene-3 (LAG-3), or T cell immunoglobulin and ITIM domain (TIGIT).

In embodiments, an immune checkpoint inhibitor (e.g., an inhibitor of PD-1 signaling, TIM-3, CTLA-4, LAG-3, or TIGIT) is a protein, antibody, antisense molecule or small molecule. In embodiments, an immune checkpoint inhibitor is an antibody.

Inhibitors of PD-1 Signaling

In embodiments, a PARP inhibitor such as niraparib is administered to a patient in combination with (e.g., simultaneously or sequentially) with a PD-1 signaling inhibitor.

Inhibitors of PD-1 signaling for use in combination therapies of the present disclosure include those that bind to and block PD-1 receptors on T cells without triggering inhibitory signal transduction, agents that bind to PD-1 ligands to prevent their binding to PD-1, agents that do both, and agents that prevent expression of genes that encode either PD-1 or natural ligands of PD-1. Compounds that bind to natural ligands of PD-1 include PD-1 itself, as well as active fragments of PD-1, and in the case of the B7-H1 ligand, B7.1 proteins and fragments. Such antagonists include proteins, antibodies, anti-sense molecules and small organics.

In some embodiments, a PD-1 signaling inhibitor binds to PD-1. In some embodiments a PD-1 signaling inhibitor binds to PD-L1 or PD-L2 (e.g., human PD-L1 or human PD-L2).

In some embodiments, a PD-1 signaling inhibitor for use in combination therapies of the present disclosure is an antibody agent. In some embodiments, a PD-1 antibody agent binds an epitope of PD-1 which blocks the binding of PD-1 to any one or more of its putative ligands. In some embodiments, a PD-1 antibody agent binds an epitope of PD-1 which blocks the binding of PD-1 to two or more of its putative ligands. In an embodiment, a PD-1 antibody agent binds an epitope of a PD-1 protein which blocks the binding of PD-1 to PD-L1 and/or PD-L2. PD-1 antibody agents of the present disclosure may comprise a heavy chain constant region (Fc) of any suitable class. In some embodiments, a PD-1 antibody agent comprises a heavy chain constant region that is based upon wild-type IgG, IgG2, or IgG4 antibodies, or variants thereof.

In some embodiments, a PD-1 signaling inhibitor is a monoclonal antibody, or a fragment thereof. In some embodiments, an antibody agent that inhibits PD-1 signaling is a PD-1 antibody or fragment thereof. Monoclonal antibodies that target PD-1 that have been tested in clinical studies and/or received marketing approval. Examples of antibody agents that target PD-1 signaling include, for example, any of the antibody agents listed in the following Table 3.

TABLE 3
Antibody agents that target PD-1

| Antibody Agent | |
|---|---|
| Target (Format) | Developer |
| Opdivo Nivolumab | Bristol-Myers Squibb |
| PD-1 (Human IgG4) | ONO |
| Keytruda Pembrolizumab | Merck |
| PD-1 (Humanized IgG4) | |
| Tecentriq | Roche |
| Atezolizumab | |
| PD-L1 (Human IgG1) | |
| Imfinzi | Astra Zeneca |
| Durvalumab | |
| PD-L1 (Human IgG1) | |
| Bavencio | Merck KGaA/Pfizer |
| Avelumab | |
| PD-L1 (Human IgG1) | |
| PDR001 | Novartis |
| PD-1 (Humanized IgG4) | |
| REGN2810 (SAR-439684) | Sanofi, Regeneron |
| PD-1 (fully human IgG4) | |
| BGB-A317 | BeiGene |
| PD-1 (Humanized IgG4) | |
| engineered to not bind FcγRI | |
| LY3300054 | Eli Lilly |
| PD-L1 | |
| BI 754091 | Boehringer Ingelheim |
| (anti-PD-1) | |
| IBI308 | Innovent Biologics |
| (anti-PD-1) | (Eli Lilly) |
| INCSHR-1210 | Incyte |
| (anti-PD-1) | |
| JNJ-63723283 | Janssen Research & |
| (anti-PD-1) | Development, LLC |
| JS-001 | Shanghai Junshi |
| (anti-PD-1) | Bioscience Co., Ltd. |
| MEDI0680 (AMP-514) | MedImmune Inc |
| anti-PD-1 (Humanized IgG4) | |
| MGA-012 | MacroGenics |
| (anti-PD-1) | |
| PF-06801591 | Pfizer |
| (anti-PD-1) | |
| REGN-2810 | Regeneron |
| (anti-PD-1) | |
| TSR-042 | TESARO |
| anti-PD-1 (Humanized IgG4) | |
| CX-072 | CytomX Therapeutics |
| anti-PD-L1 | |
| FAZ053 | Novartis |
| anti-PD-Li | |
| PD-L1 millamolecule | Bristol-Myers Squibb |

PD-1 signaling inhibitors include those that bind to and block PD-1 receptors on T cells without triggering inhibitory signal transduction, agents that bind to PD-1 ligands to prevent their binding to PD-1, agents that do both and agents that prevent expression of genes that encode either PD-1 or natural ligands of PD-1. In some embodiments, an agent that inhibits PD-1 signaling is an antibody agent. Anti-PD-1 antibody agents can include any polypeptide or polypeptide complex that includes immunoglobulin structural elements sufficient to confer specific binding. Exemplary antibody agents include, but are not limited to, monoclonal antibodies, polyclonal antibodies, antibody fragments such as Fab fragments, Fab′ fragments, F(ab′)2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE® s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR® s. In some embodiments, an antibody agent that inhibits PD-1 signaling is a monoclonal antibody or a derivative thereof. In some embodiments, an antibody agent that inhibits PD-1 signaling is a PD-1 antibody, a PD-L1 antibody, or a derivative thereof. PD-1 and PD-L1 antibodies include, for example, atezolizumab, avelumab, BGB-A317, BI 754091, CX-072, durvalumab, FAZ053, IBI308, INCSHR-1210, JNJ-63723283, JS-001, LY3300054, MEDI-0680, MGA-012, nivolumab, PD-L1 millamolecule, PDR001, pembrolizumab, PF-06801591, REGN-2810, TSR-042, any of the antibodies disclosed in WO2014/179664, and any derivatives thereof. In embodiments, an agent includes combinations of agents that inhibit PD-1 signaling.

In embodiments, administration of a particular dose or cycle of a PARP inhibitor is separated in time from a particular dose or cycle of an agent that inhibits PD-1 signaling by a time period having a length that may be, for example, 1-minute, 5-minutes, 30-minutes, 1-hour, 2-hours, 5-hours, 10-hours, 12-hours, 24-hours, 48-hours, 72-hours, 96-hours, 1-week, 2-weeks, or more weeks. In some embodiments, the range may be bounded by a lower limit and an upper limit, the upper limit being larger than the lower limit. In some embodiments, the lower limit may be about 1-minute, about 5-minutes, about 15-minutes, about 30-minutes, about 45-minutes, about 1-hour, about 2-hours, about 4-hours, about 6-hours, about 12-hours, about 24-hours, about 48-hours, about 72-hours, about 96-hours, or about 1-week. In some embodiments, the upper limit may be about 2-weeks, about 3-weeks, about 4-weeks, about 5-weeks, about 6-weeks, about 8-weeks, or about 12-weeks. In some embodiments, the administration of a particular dose of a PARP inhibitor is separated in time from a particular dose of an agent that inhibits PD-1 signaling by a time period within the range of about 1-minute to about 12-weeks. In some embodiments, the range may be about 1-minute to about 8-weeks. In some embodiments, the range may be about 1-minute to about 6-weeks. In some embodiments, the range may be about 1-minute to about 4-weeks. In some embodiments, the range may be about 1-minute to about 2-weeks. In some embodiments, the range may be about 1-minute to about 1-week. In some embodiments, the range may be about 1-minute to about 96-hours. In some embodiments, the range may be about 1-minute to about 72-hours. In some embodiments, the range may be about 1-minute to about 48-hours. In some embodiments, the range may be about 1-minute to about 24-hours. In some embodiments, the range may be about 1-minute to about 12-hours. In some embodiments, the range may be about 1-minute to about 8-hours. In some embodiments, the range may be about 1-minute to about 4-hours. In some embodiments, the range may be about 1-minute to about 2-hours. In some embodiments, the range may be about 1-minute to about 1-hour. In some embodiments, the range may be about 1-minute to about 11 minutes.

In some embodiments, combination therapy with a PARP inhibitor and a PD-1 signaling inhibitor is administered to a patient or population of subjects who has exhibited response to prior therapy. In some embodiments, the patient or population of subjects has exhibited response to prior therapy with a chemotherapeutic agent. In some such embodiments, the chemotherapeutic agent is a platinum agent. In some embodiments, a platinum-based agent is selected from cisplatin, carboplatin, oxaliplatin, nedaplatin, triplatin tetranitrate, phenanthriplatin, picoplatin, or satraplatin.

In some embodiments, the regimen comprises at least one oral dose of a PARP inhibitor. In some embodiments, the regimen comprises a plurality of oral doses. In some embodiments, the regimen comprises once daily (QD) dosing. In some embodiments, a PARP inhibitor is administered on the first day of a 21-day cycle upon completion of infusion with a PD-1 signaling inhibitor. In some embodiments, a PARP inhibitor is administered daily throughout the regimen cycle at the same time every day. In some embodiments the same time every day is preferably in the morning.

In some embodiments, the regimen comprises of one infusion of a PD-1 signaling inhibitor per regimen cycle. In some embodiments, the regimen comprises of one, 30-minute infusion of a PD-1 signaling inhibitor per regimen cycle. In some embodiments, the regimen comprises of one, 30-minute infusion of a PD-1 signaling inhibitor on the first day of each regimen cycle.

In some embodiments, the regimen comprises at least one 2-week to 8-week cycle. In some embodiments, the regimen comprises a plurality of 2-week to 8-week cycles. In some embodiments, the regimen comprises one 2-week to 8-week cycle. In some embodiments, the regimen comprises two 2-week to 8-week cycles. In some embodiments, the regimen comprises three or more 2-week to 8-week cycles. In some embodiments, the regimen comprises continuous 2-week to 8-week cycles.

In some embodiments, the regimen comprises at least one 28-day cycle. In some embodiments, the regimen comprises a plurality of 28-day cycles. In some embodiments, the regimen comprises one 28-day cycle. In some embodiments, the regimen comprises two 28-day cycles. In some embodiments, the regimen comprises three or more 28-day cycles. In some embodiments, the regimen comprises continuous 28-day cycles.

In some embodiments, the regimen comprises at least one 21-day cycle. In some embodiments, the regimen comprises a plurality of 21-day cycles. In some embodiments, the regimen comprises one 21-day cycle. In some embodiments, the regimen comprises two 21-day cycles. In some embodiments, the regimen comprises three or more 21-day cycles. In some embodiments, the regimen comprises continuous 21-day cycles.

In some embodiments, the regimen comprises a single infusion of at least 200 mg of a PD-1 signaling inhibitor. In some embodiments, the regimen comprises a single infusion of a PD-1 signaling inhibitor over a time period of at least 25-minutes, 30-minutes, 35-minutes, 40-minutes, or more. In some embodiments, the range may be bounded by a lower limit and an upper limit, the upper limit being larger than the lower limit. In some embodiments, the lower limit may be about 25-minutes, or about 30-minutes. In some embodiments, the upper limit may be about 35-minutes or about 40-minutes. In some embodiments, the range may be about 25-minutes to about 40-minutes. In some embodiments, the range may be about 25-minutes to about 35-minutes. In some embodiments, the range may be about 25-minutes to about 30-minutes. In some embodiments a PD-1 signaling inhibitor (e.g., pembrolizumab) is administered through intravenous (IV) infusion. In some embodiments an intravenous dose of a PD-1 signaling inhibitor (e.g., pembrolizumab) is administered in one or more unit dosage forms.

### EXAMPLES

### Example 1. NOVA Example

Treatment of Platinum Sensitive Ovarian Cancer

In NOVA, platinum-sensitive recurrent ovarian cancer patients who were in response following platinum-based treatment were prospectively randomized to receive either niraparib or placebo. Two cohorts were treated: the germline BRCA mutant positive cohort (gBRCAmut) and the non-germline BRCA cohort (non-gBRCAmut). Therefore, the gBRCAmut cohort of NOVA was designed to prospectively test the treatment effect of niraparib versus placebo in patients with platinum-sensitive recurrent ovarian cancer who were in response after platinum-based treatment. Patients in this cohort were germline BRCA mutation carriers as assessed by the FDA-approved Integrated BRACAnalysis test. Patients in the non-gBRCAmut were negative in the FDA-approved Integrated BRACAnalysis test.

The double-blind, 2:1 randomized, study evaluated niraparib as maintenance therapy in patients with recurrent and/or platinum sensitive ovarian cancer who had either gBRCAmut or a tumor with high-grade serous histology. The study compared maintenance treatment with niraparib with to placebo and is evaluating the efficacy of niraparib as maintenance therapy in patients who have recurrent ovarian cancer as assessed by the prolongation of progression-free survival (PFS). This objective is independently evaluated in a cohort of patients with germline BRCA mutation (gBRCAmut) and in a cohort of patients who have high grade serous or high grade predominantly serous histology but without such gBRCA mutations (non-gBRCAmut). Some patients in the non-gBRCAmut cohort have been reported to share distinctive DNA repair defects with gBRCAmut carriers, a phenomenon broadly described as “BRCAness.” (See Turner, N., A. Tutt, and A. Ashworth, Hallmarks of ‘BRCAness’ in sporadic cancers”, Nat. Rev. Cancer 4(10): 814-19, (2004)). Recent studies have suggested that homologous recombination deficiency (HRD) in epithelial ovarian cancer (EOC) is not solely due to germline BRCA1 and BRCA2 mutations. (See Hennessy, B. T. et al. Somatic mutations in BRCA1 and BRCA2 could expand the number of patients that benefit from poly (ADP ribose) polymerase inhibitors in ovarian cancer. Journal of clinical oncology: official journal of the American Society of Clinical Oncology 28, 3570-3576, (2010); TCGA “Integrated genomic analyses of ovarian carcinoma”, Nature 474(7353): 609-15 (2011); and Dann R B, DeLoia J A, Timms K M, Zorn K K, Potter J, Flake D D 2nd, Lanchbury J S, Krivak T C. “BRCA 1/2 mutations and expression: Response to platinum chemotherapy in patients with advanced stage epithelial ovarian cancer”, Gynecol Oncol. 125(3): 677-82, (2012)). Non-BRCA deficiencies in homologous recombination DNA repair genes could also enhance tumor cell sensitivity to PARP inhibitors. Accordingly, HRD is used as a tumor biomarker classifier to be evaluated.

Patients enrolled in this study had received at least two platinum-based regimens, had a response (complete or partial) to their last regimen, and had no measurable disease >2 cm and normal cancer antigen CA125 (or >90% decrease) following their last treatment. Patients were assigned to one of two independent cohorts—one with deleterious gBRCA mutations (gBRCAmut) and the other with high-grade serous histology but without such gBRCA mutations (non-gBRCAmut) according to the following criteria (Table 4):

TABLE 4
NOVA Cohorts

| Mutation Status | Cohort for Randomization |
|---|---|
| Positive for a Deleterious Mutation | gBRCA^{mut} cohort |
| Genetic Variant, Suspected Deleterious | gBRCA^{mut} cohort |
| Genetic Variant, Favor Polymorphism | non-gBRCA^{mut} cohort |
| Genetic Variant of Uncertain Significance | non-gBRCA^{mut} cohort |
| No Mutation Detected | non-gBRC^{mut} cohort |

Patients were also assessed for HRD status and were further classified as HRD positive (HRDpos) or HRD negative (HRDneg).

Study treatment was dispensed to patients on Day 1 and every cycle (28 days) thereafter until the patient discontinued study treatment. Study treatment was administered orally once daily continuously. Three capsules of 100 mg strength were taken at each dose administration. Clinic visits occurred in each cycle (every 4 weeks 3 days). Response evaluation criteria in solid tumors (RECIST) tumor assessment via computed tomography (CT) or magnetic resonance imaging (MRI) scan of abdomen/pelvis and clinically indicated areas was required at the end of every 2-cycles (8-weeks with a window of 7 days from date of visit) through Cycle 14, then at the end of every 3-cycles (12-weeks with a window of 7 days from date of visit) until progression.

Patients were assessed by the prolongation of progression-free survival (PFS). More specifically, progression was determined if at least one of the following criteria is met: 1) tumor assessment by CT/MRIunequivocally shows progressive disease according to RECIST 1.1 criteria; 2) additional diagnostic tests (e.g. histology/cytology, ultrasound techniques, endoscopy, positron emission tomography) identify new lesions or determine existing lesions qualify for unequivocal progressive disease and CA-125 progression according to Gynecologic Cancer Intergroup (GCIG)-criteria (see Rustin et al., Int J Gynecol Cancer 2011; 21: 419-423); 3) definitive clinical signs and symptoms of PD unrelated to non-malignant or iatrogenic causes ([i] intractable cancer-related pain; [ii] malignant bowel obstruction/worsening dysfunction; or [iii] unequivocal symptomatic worsening of ascites or pleural effusion and CA-125 progression according to GCIG-criteria. Response Evaluation Criteria in Solid Tumors (RECIST) was used for tumor assessment via a computed tomography (CT) or magnetic resonance imaging (MRI) scan of abdomen/pelvis and clinically indicated areas, which was required at the end of every 2-cycles (8-weeks) through cycle 14 (56-weeks), and then at the end of every 3-cycles (12-weeks) until progression.

Patients continued to receive their assigned treatment until disease progression, unacceptable toxicity, death, withdrawal of consent, and/or lost to follow-up. Dose interruption and/or reduction were available at any time for any grade toxicity considered intolerable by the patient.

Identification of Non-BRCA1/2 HRR Deficiencies

Following completion of the NOVA study, formalin-fixed, paraffin-embedded (FFPE) archival tumor samples from NOVA patients were retrospectively analyzed using a pre-specified gene panel.

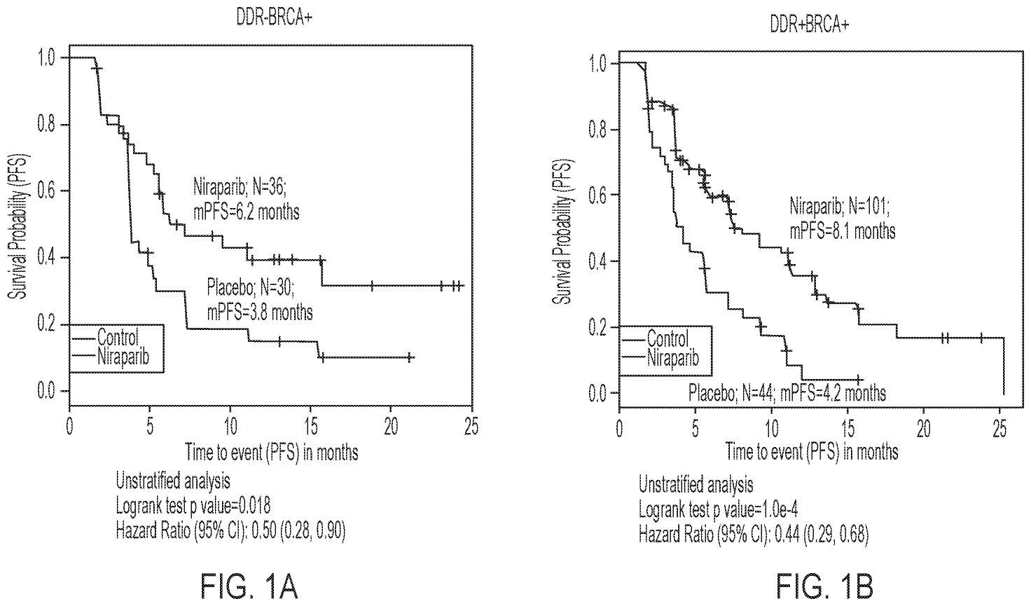

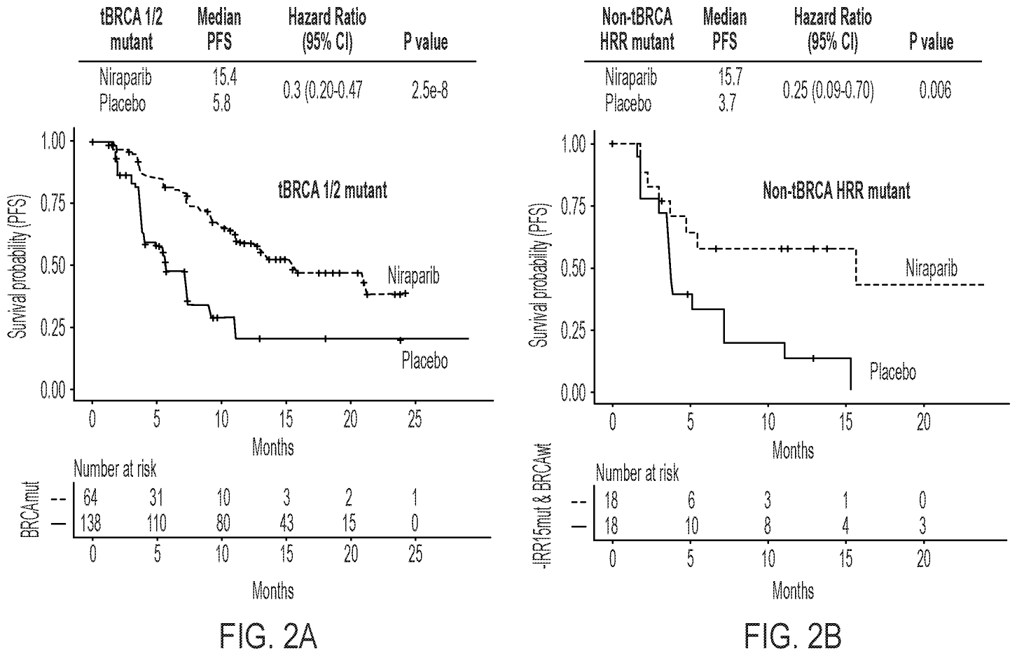

In the analysis, NOVA patient samples were tested using a gene panel that reports the mutation status of 31 DNA damage repair (DDR) genes. As shown in FIGS. 1A-1B, mutations in any of the 31 DDR genes was not predictive of niraparib response in BRCA wild type patients. However, when using Cox models to evaluate a subpanel of genes (ATM, ATR, BAP1, BARD1, BLM, BRIP1, MRE11A, NBN, PALB2, RAD51, RAD51B, RAD51C, RAD51D, RAD52, RAD54L, and XRCC2), it was discovered that patients with at least one non-BRCA HRR mutation experienced similar benefits to niraparib treatment as compared to patients having a BRCA1/2 mutation, as shown in FIGS. 2A and 2B and Table 5.

TABLE 5
Treatment of Patients having BRCA and Non-BRCA HRR Mutations

| | | Median | | |
|---|---|---|---|---|
| | | Progression | | |
| Mutation | | Free Survival | Hazard Ratio | |
| Status | Treatment | (PFS) | (95% CI) | P value |
| tBRCA1/2 | Niraparib | 15.4 | 0.3 (0.20-0.47) | 2.5e−8 |
| mutant | Placebo | 5.8 | | |
| Non-tBRCA1/2 | Niraparib | 15.7 | 0.25 (0.09-0.70) | 0.006 |
| HRR mutant | Placebo | 3.7 | | |

Non-tBRCA HRR = ATM, ATR, BAP1, BARD1, BLM, BRIP1, MRE11A, NBN, PALB2, RAD51, RAD51B, RAD51C, RAD51D, RAD52, RAD54L, and XRCC2

### Example 2. Monotherapies and Combination Therapies for Treatment of Lung Cancer

Treatment of Non-Small Cell Lung Cancer with Niraparib Including Combination with a PD-1 Signaling Inhibitor

A PARP inhibitor (e.g., niraparib) is administered to three groups of cancer patients having lung cancer, including non-small cell lung cancer (NSCLC) as shown in Table 6.

TABLE 6
Treatment of Patients having Non-Small Cell Lung Cancer (NSCLC)

| Cohort | Cancer | Treatment |
|---|---|---|
| 1 | NSCLC High | Combination Therapy: Niraparib (niraparib |
| | PD-L1 Expressing | tosylate monohydrate) and a biological PD-1 |
| | (TPS greater or | inhibitor |
| | equal to 50%) | |
| 2 | NSCLC Low | Combination Therapy: Niraparib (niraparib |
| | PD-L1 Expressing | tosylate monohydrate) and a biological PD-1 |
| | (TPS 1-49%) | inhibitor |
| 3 | Squamous | Monotherapy: Niraparib (niraparib tosylate |
| | NSCLC | monohydrate) |

TPS = tumor proportion score

Eligible patients for inclusion in Cohorts 1, 2, and 3 include adults of at least 18 years of age having a histologically- or cytologically-proven advanced (unresectable) or metastatic NSCLC as defined as stage IIIB (positive supraclavicular lymph nodes) not amenable to definitive chemoradiotherapy or stage IV NSCLC. A selected patient will have measurable disease (e.g., by RECIST v1.1). A patient to be selected for Cohort 1 must have tumors with high PD-L1 expression (TPS≥50%) per local assessment; with no known EGFR sensitizing mutation and/or ROS-1 or ALK translocations, and no prior systemic chemotherapy or PD-1/PD-L1 inhibitor treatment for metastatic NSCLC. A patient to be selected for Cohort 2 must have tumors with PD-L1 expression (TPS between 1% and 49%) per local assessment, with no known EGFR-sensitizing mutation and/or ROS-1 or ALK translocation, and no prior systemic chemotherapy or PD-1/PD-L1 inhibitor treatment for metastatic NSCLC. A patient to be selected for Cohort 3 must have metastatic sqNSCLC and have progressed after both prior platinum-based chemotherapy and prior PD-1 or PD-L1 inhibitor treatment

For a selected cancer patient, a PARP inhibitor (e.g., niraparib) can be administered according to any regimen described herein. For example, a patient in any of Cohorts 1, 2, and 3, is orally administered a PARP inhibitor (e.g., niraparib) according to a regimen comprising once daily (QD) dosing. For example, a cancer patient in Cohort 1, Cohort 2, or Cohort 3 receiving PARP inhibitor treatment is administered niraparib as an oral dose (e.g., an amount of niraparib tosylate monohydrate in an amount equivalent to 200 mg niraparib free base).

For a cancer patient in Cohort 1 or Cohort 2 who receives both PARP inhibitor treatment and PD-1 inhibitor treatment, treatment also comprises administering (e.g., via intravenous administration) a biological PD-1 inhibitor (e.g., an agent that is a monoclonal antibody). Administering of a biological PD-1 inhibitor can be according to any of the regimens described herein.

For a cancer patient who receives both PARP inhibitor treatment and PD-1 inhibitor treatment, identification of a non-BRCA1/2 HRR gene deficiency as described herein (e.g., a deficiency in any of the genes of Tables 1 and 2 such as a subpanel of genes that includes any or all of ATM, ATR, BAP1, BARD1, BLM, BRIP1, MRE11A, NBN, PALB2, RAD51, RAD51B, RAD51C, RAD51D, RAD52, RAD54L, and XRCC2), can be predictive of patient response (e.g., a beneficial response such as complete response or partial response) to the combination therapy.

### Example 3. Elucidation of Non-BRCA Lesions Driving PARP Synthetic Lethality

The relative contribution of the loss of BRCA and non-BRCA HRR genes to PARP synthetic lethality in additional indications other than ovarian and breast was also evaluated. To this end, CRISPR/Cas9 technology was utilized to knock-out either the single or both alleles of 11 clinically-relevant HR genes in two different genetic backgrounds. Niraparib sensitivity was assessed in HRR11 KO isogenic cell lines as well as in 77 PDX models with monoallelic and bi-allelic deleterious mutations in HR genes across 17-tumor types. Notably, while bi-allelic mutations were found to cause the highest degree of niraparib sensitivity in lung, gastric, pancreatic, liver, cervical, uterine cancer and melanoma, some monoallelic HR mutations were also found to be sensitive to niraparib. Overall, such data provides evidence that niraparib sensitivity can extend beyond BRCA genes in multiple indications in addition to ovarian and breast cancer.

HRR KO Isogenic Cell Line Generation and Sensitivity Evaluation:

CRISPR/Cas9 technology was used to knock-out either the single or both alleles of 11 clinically-relevant HR genes in two different genetic backgrounds, using Dld-1 and HeLa cell lines. Niraparib sensitivity was assessed in HRR11 KO isogenic cell lines with homozygous and heterozygous KO of 11 HRR genes in Dld-1 cell line (HeLa HRR KO cell line niraparib sensitivity TBD, early CY2019). Niraparib sensitivity was assessed using a 3D clonogenic assay setting in a 96 well format with colony count based on image analysis as read-out testing 10-point dose titrations of niraparib. Compounds were added 24 h after cell seeding, and then every 3- to 4-days (2-times a week) during the incubation period (for 13-days incubation period).

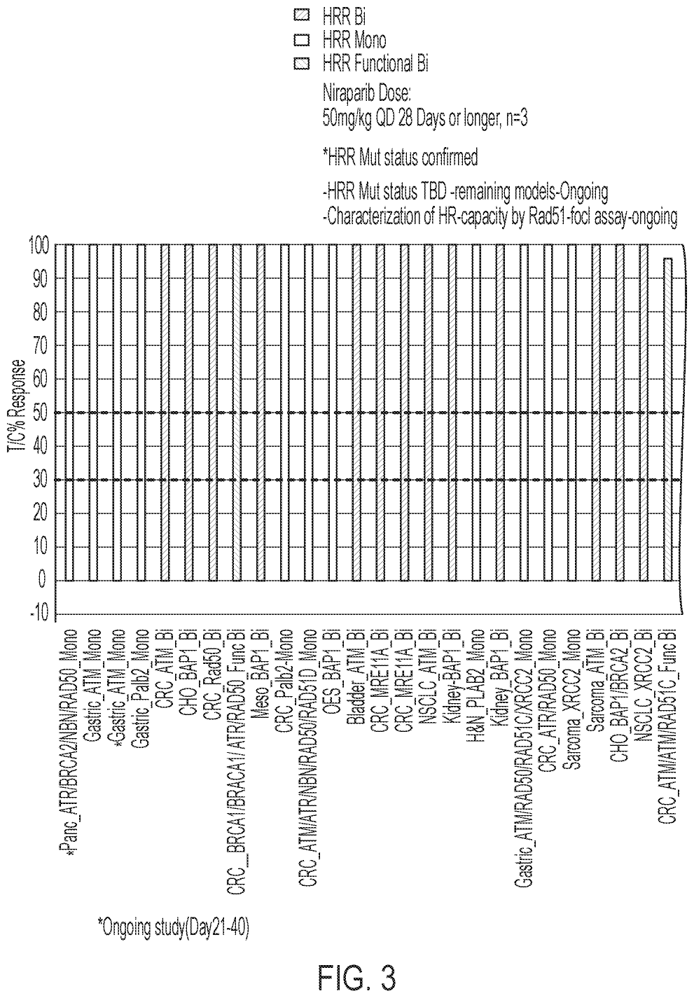

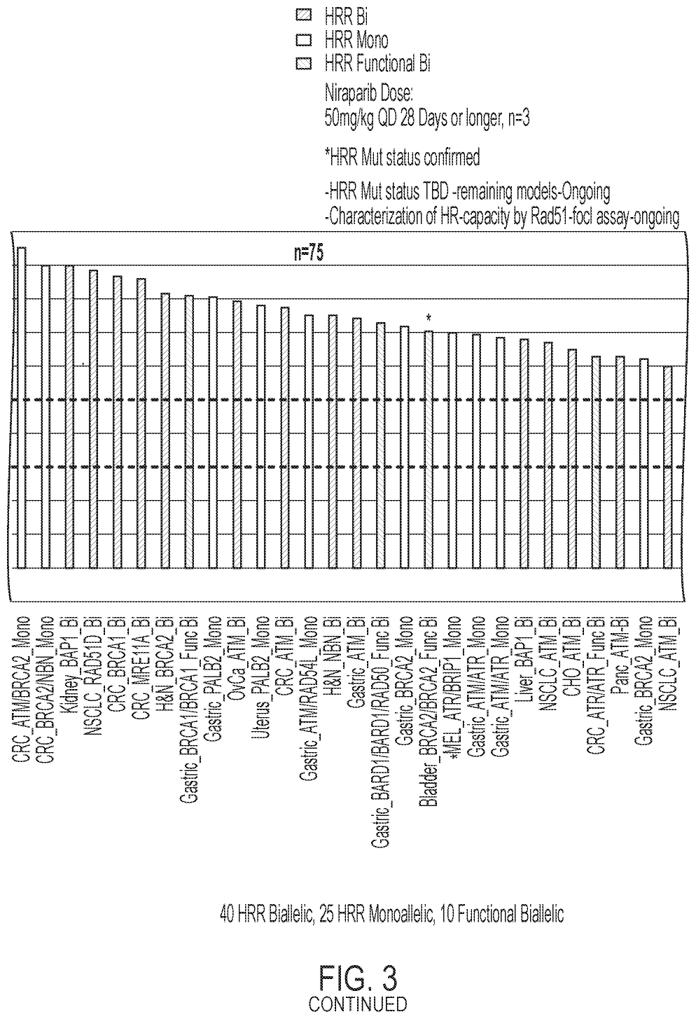

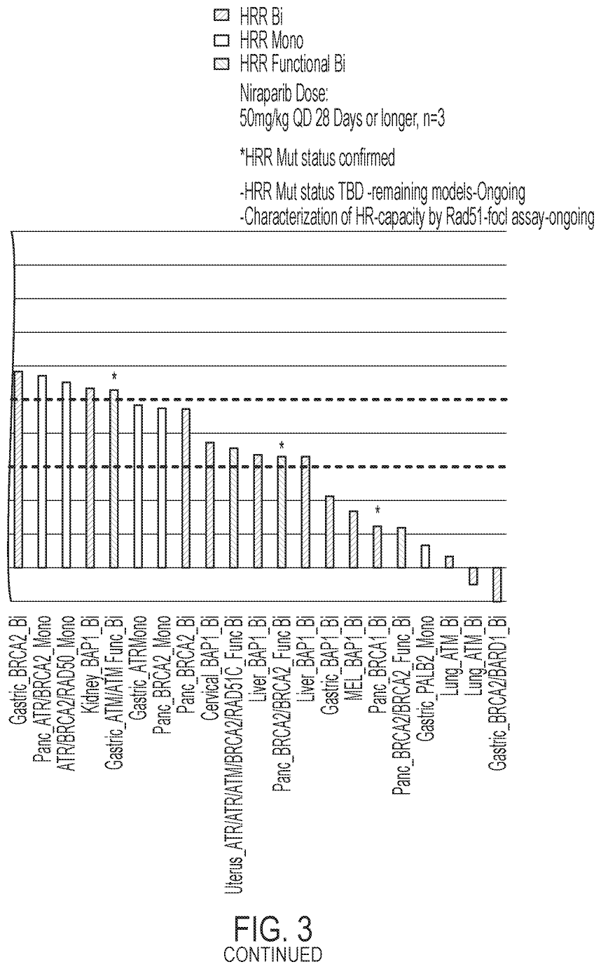

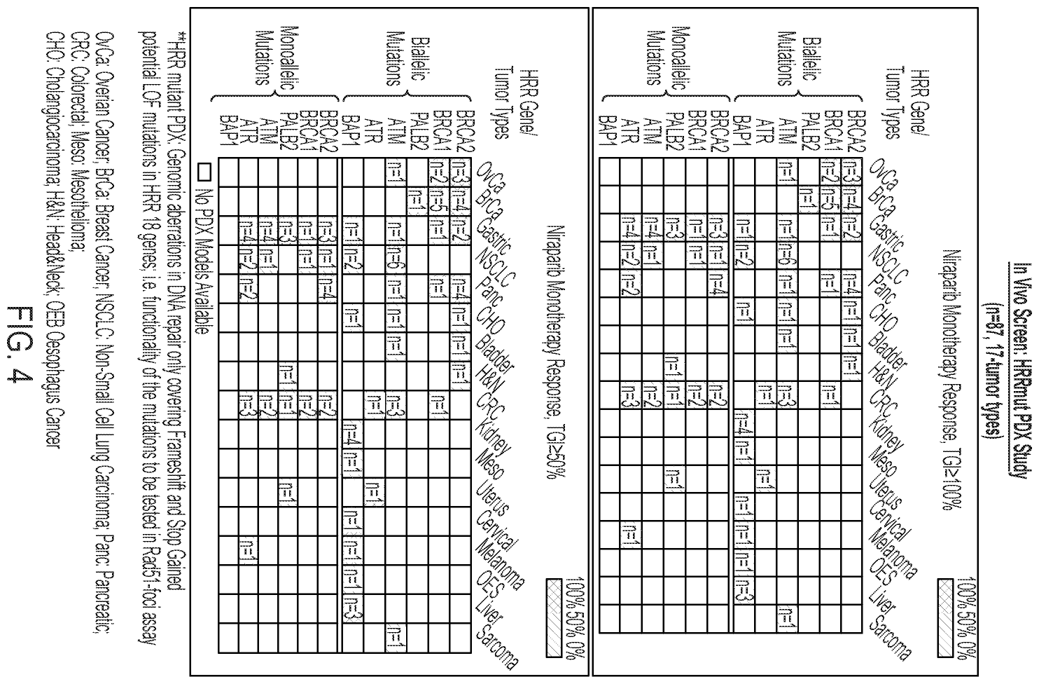

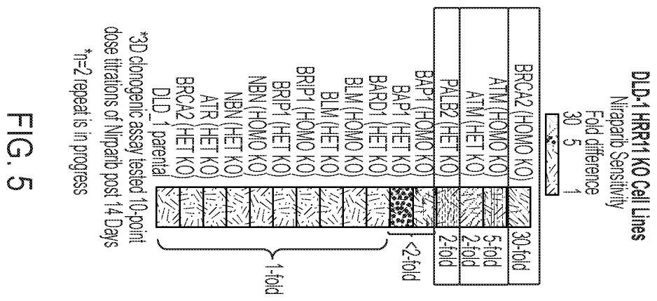

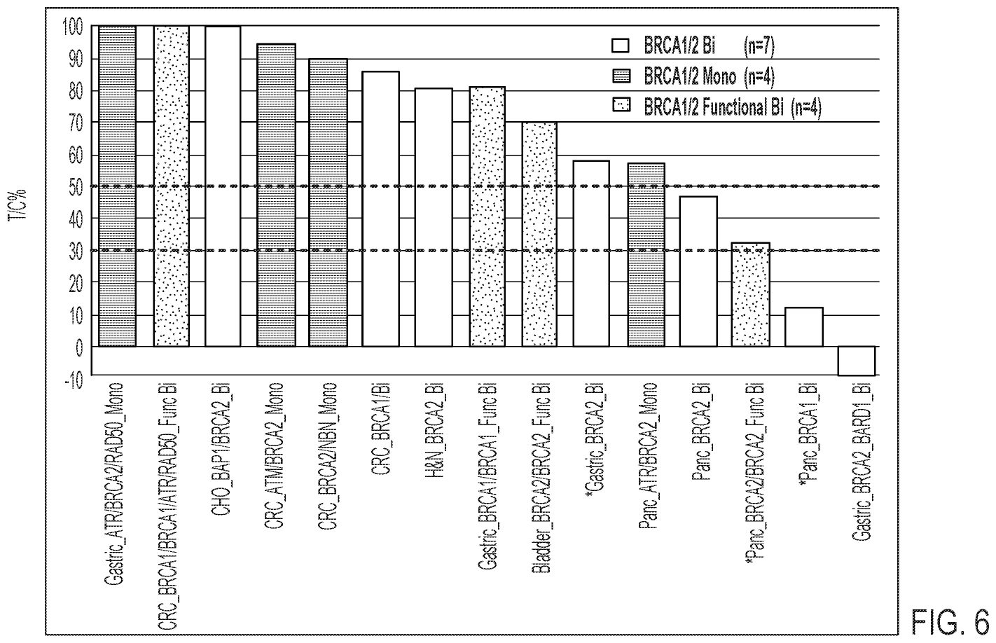

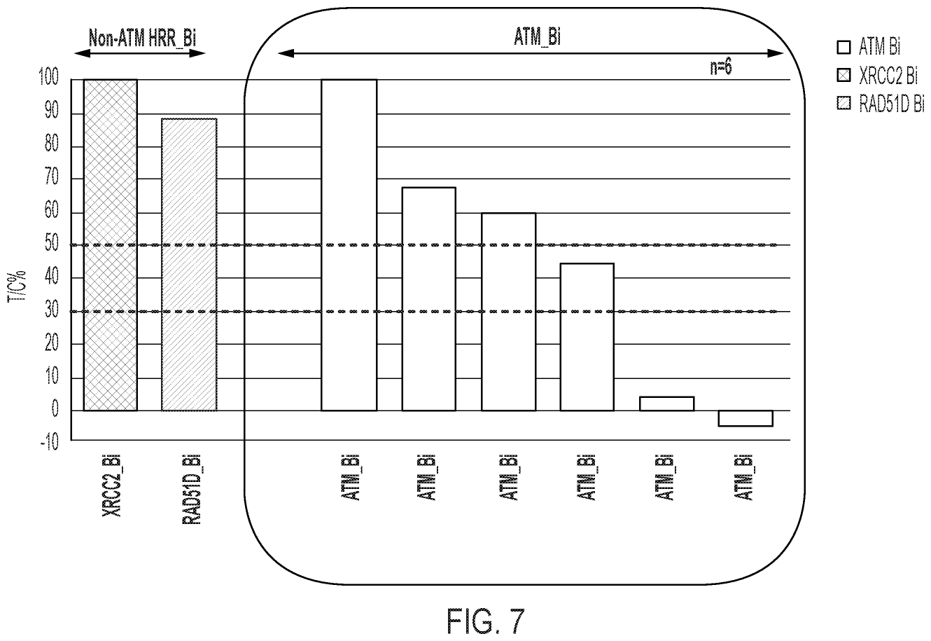

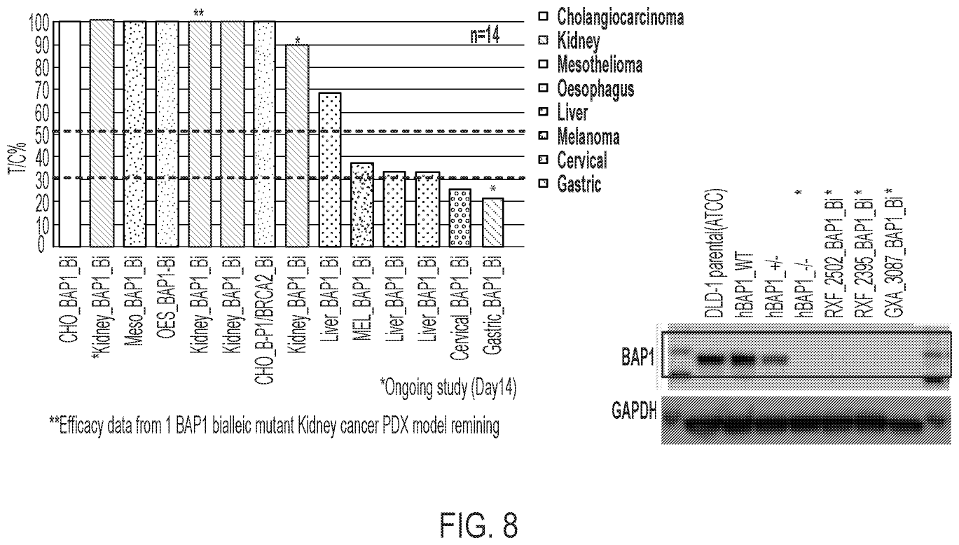

Niraparib sensitivity was observed in PDX models containing ATM, BAP1, and BRCA bi-allelic mutations, with responses based on the tumor growth inhibition (T/C) ratio (FIG. 3). Bi-allelic mutations in BRCA1, BRCA2, PALB2 and ATM demonstrated the strongest niraparib sensitivity (see FIGS. 4 and 5) based on observed total growth inhibition (TGI). FIG. 6 shows that 43% BRCA2 bi-allelic mutant PDX models demonstrated moderate sensitivity to niraparib, with TGI≥50% (80% OvCa PDX models demonstrated >100% TGI). 14% ATM bi-allelic mutant PDX models demonstrated moderate sensitivity to niraparib, with TGI≥50% (FIG. 5). FIG. 7 shows 33% of ATM bialllelic mutant NSCLC PDX models showed strong sensitivity to niraparib, with TGI>70%. None of the ATM monoallelic mutant PDX models (0/6) demonstrated TGI≥50%. 17% PALB2 monoalleic mutant PDX models (1/6) demonstrated strong sensitivity to niraparib, with TGI 93% (FIG. 5). FIG. 8 shows 36% of models (across 5-tumor types) were sensitive to niraparib with ≥50% TGI.

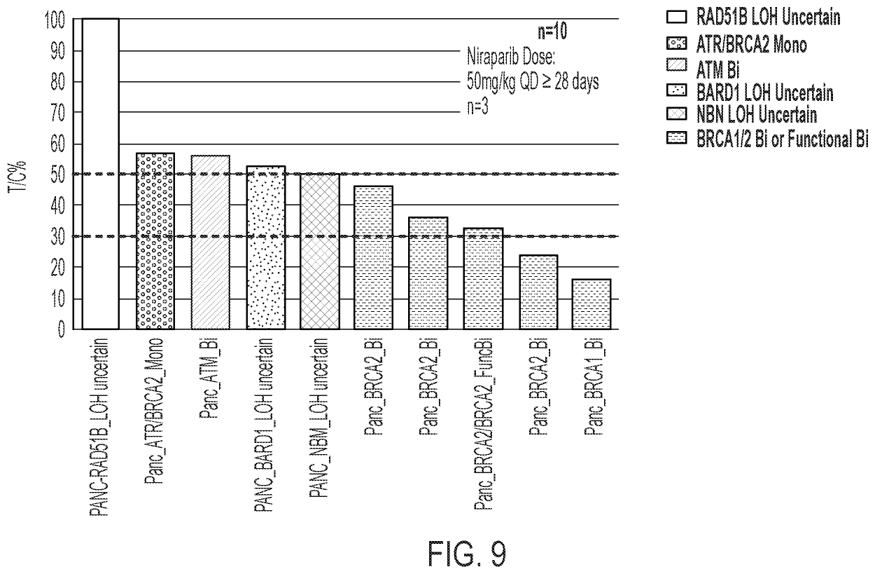

Preclinical and clinical data provides strong evidence to support treating HRR mutant pancreatic patients with niraparib (FIG. 9).

HRR bi-allelic mutations cause PARP sensitivity across multiple cancer types. Efficacy data using HRR bi-allelic mutant NSCLC, pancreatic, gastric PDX models provide supportive preclinical POC data for an HRR mutant basket trial. Some mono-allelic HR mutations were also found to be sensitive to niraparib.

### Example 4. Exploratory Analysis of Mutations in Circulating Tumor DNA for Patients with a Complete or Partial Response to Platinum-Based Chemotherapy in Recurrent Ovarian Cancer

Analyses of circulating tumor DNA (ctDNA) were used to assess the mutation status of HRR genes that can be predictive of niraparib response.

Remnant plasma samples from 104 patients, originally collected within 8 weeks after completion of platinum regimen and before or during niraparib treatment for pharmacokinetic study, were selected for ctDNA analyses based on tumor biomarker or CR/PR status. Following patient de-identification steps, ctDNA was tested using an HRR assay that includes a panel of genes relevant to the DNA damage repair (DDR) pathway and additional genes related to ovarian cancer biology: TP53 and RB1. Assay performance was evaluated in suboptimal PK plasma samples and the mutant allele fraction (MAF) of HRR genes or the entire panel was assessed in both CR and PR patients. The mutation status from blood-based results were compared to tumor-based test results.

### Example 5. Targeting Homologous Recombination Repair Defects in Lung Cancer

To investigate the potential of targeting the DNA Damage Response (DDR) pathway in lung cancer, as an alternative treatment approach for these patients we sought to identify whether functionally relevant HRR (Homologous Recombination Repair)-defects could be synthetically lethal with niraparib monotherapy in NSCLC xenograft tumors.

Niraparib sensitivity was evaluated in 57 NSCLC PDX models containing both BRCA and non-BRCA HRR mutations (n=17) as well as HRR WT models (n=40). This analysis demonstrated that niraparib sensitive models include both HRR mutant and HRR WT lung tumors. Amongst the PDX models containing a bi-allelic HRR mutation, the ATM bi-allelic mutant models were sensitive to niraparib (2 out of 8). Surprisingly, 7.5% (3 out of 40) of the HRR WT PDX models were sensitive to niraparib.

### EQUIVALENTS

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or the entire group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. The publications, websites and other reference materials referenced herein to describe the background of the invention and to provide additional detail regarding its practice are hereby incorporated by reference.

1.-367. (canceled)

368. A method of treating cancer in a human, the method comprising administering to the human in need thereof a therapeutically effective amount of a poly (ADP-ribose) polymerase (PARP) inhibitor, or a pharmaceutically acceptable salt thereof, wherein the human has a deficiency in at least one gene involved in the homologous recombination repair (HRR) pathway, wherein at least one gene involved the HRR pathway is not BRCA1 or BRCA2.

369. The method according to claim 368, wherein the human has a deficiency in at least one gene selected from the group consisting of RFC2, XRCC6, POLD2, PCNA, RPA1, RPA2, ERCC3, UNG, ERCC5, MLH1, LIG1, MSH6, POLD4, RFC5, DDB2 /// LHX3, POLD1, FANCG, POLB, XRCC1, MPG, ERCC1, TDG, FANCA, RFC4, RFC3, APEX2, RAD1, EXO1, FEN1, MLH3, MGMT, RAD51, XRCC4, RECQL, ERCC8, FANCC, OGG1, MRE11A, RAD52, WRN, XPA, BLM, MSH3, POLE2, RAD51C, LIG4, ERCC6, LIG3, RAD17, XRCC2, MUTYH, RFC1, RAD50, DDB1, XRCC5, PARP1, POLE3, XPC, MSH2, RPA3, MBD4, NTHL1, PMS2 /// PMS2CL, UNG2, APEX1, ERCC4, RECQL5, MSH5, POLD3, ERCC2, RECQL4, PMS1, ZFP276, POLE, XRCC3, NBN, SMUG1, FANCF, NEIL1, FANCE, ATM, ATR, BAP1, BARD1, BRIP1, PALB2, RAD51B, RAD51D, RAD54L, TP53, RB1, and combinations thereof.

370. The method according to claim 368, wherein the cancer is a recurrent cancer.

371. The method according to claim 370, wherein the human has undergone at least one cycle of a platinum-based chemotherapy.

372. The method according to claim 371, wherein the human has a complete or a partial response to the most recent cycle of platinum-based chemotherapy.

373. The method according to claim 368, wherein the deficiency in at least one gene involved in the HRR pathway is identified by analyzing cancer cells, wherein the cancer cells are circulating tumor cells.

374. The method according to claim 368, wherein a deficiency in the at least one gene involved in the HRR pathway is identified by analyzing cell-free DNA.

375. The method according to claim 368, wherein the PARP inhibitor is administered in the absence of determining the BRCA status of the human.

376. The method according to claim 368, wherein the PARP inhibitor is administered prior to determining the BRCA status of the human.

377. The method according to claim 368, wherein the PARP inhibitor is administered independent of the BRCA status of the human.

378. The method according to claim 368, wherein the human has no germline mutation in BRCA1 and/or BRCA2.

379. The method according to claim 368, wherein the human has no sporadic mutation in BRCA1 and/or BRCA2.

380. The method according to claim 368, wherein the cancer is selected from the group consisting of: bladder cancer, breast cancer, cancer of the fallopian tube(s), cholagiocarcinoma, colon adenocarcinoma, endometrial cancer, esophageal cancer, Ewing's sarcoma, gastric cancer, kidney clear cell cancer, lung cancer, mesothelioma, ovarian cancer, pancreatic cancer, peritoneal cancer, prostate cancer, uterine endometrial cancer, or uveal melanoma.

381. The method according to claim 368, wherein the cancer is breast cancer or triple negative breast cancer.

382. The method according to claim 368, further comprising administering one or more additional therapeutic agents.

383. The method according to claim 382, wherein the one or more additional therapeutic agents comprises an immune checkpoint inhibitor.

384. The method according to claim 383, wherein the immune checkpoint inhibitor is an agent that inhibits programmed death-1 protein (PD-1) signaling, T-cell immunoglobulin domain and mucin domain 3 (TIM-3), cytotoxic T-lymphocyte-associated protein 4 (CTLA-4), lymphocyte activation gene-3 (LAG-3), or T cell immunoglobulin and ITIM domain (TIGIT).

385. The method according to claim 384, wherein the PD-1 signaling inhibitor is selected from the group consisting of BGB-A317, BI 754091, IBI308, INCSHR-1210, JNJ-63723283, JS-001, MEDI-0680, MGA-012, nivolumab, PDR001, pembrolizumab, PF-06801591, REGN-2810, TSR-042, atezolizumab, avelumab, CX-072, durvalumab, FAZ053, LY3300054, or PD-L1 millamolecule, or derivatives thereof.

386. The method according to claim 384, wherein the PD-1 signaling inhibitor is an anti-PD-L1/L2 agent.

387. The method of claim 386, wherein the anti-PD-L1 agent is atezolizumab, avelumab, CX-072, durvalumab, FAZ053, LY3300054, PD-L1 millamolecule, or derivatives thereof.

388. The method according to claim 368, wherein the PARP inhibitor is selected from the group consisting of: ABT-767, AZD 2461, BGB-290, BGP 15, CEP 8983, CEP 9722, DR 2313, E7016, E7449, fluzoparib, IMP 4297, INO1001, JPI 289, JPI 547, monoclonal antibody B3-LysPE40 conjugate, MP 124, niraparib, NU 1025, NU 1064, NU 1076, NU1085, olaparib, ONO2231, PD 128763, R 503, R554, rucaparib, SBP 101, SC 101914, Simmiparib, talazoparib, veliparib, WW 46, 2-(4-(trifluoromethyl)phenyl)-7,8-dihydro-5H-thiopyrano[4,3-d]pyrimidin-4-ol, and salts or derivatives thereof.

389. The method according to claim 368, wherein the PARP inhibitor is niraparib free base or a pharmaceutically acceptable salt thereof.

390. The method according to claim 368, wherein the PARP inhibitor is niraparib tosylate monohydrate.
